SEQUENCE LISTING (232 sequences)

SEQ ID NO: 1 - DNA, 24 bp, Artificial Sequence
  Synthetic Probe
tcctggcctgccatcacggttgga

SEQ ID NO: 2 - DNA, 24 bp, Artificial Sequence
  Synthetic Primer
gtgctttctggagggtctactatg

SEQ ID NO: 3 - DNA, 21 bp, Artificial Sequence
  Synthetic Primer
ggtgggtcttcaatgcggatc

SEQ ID NO: 4 - DNA, 26 bp, Artificial Sequence
  Synthetic Probe
actgatgagtcccactgtgctgagca

SEQ ID NO: 5 - DNA, 23 bp, Artificial Sequence
  Synthetic Primer
gtagagtggaaaaggagcattgg

SEQ ID NO: 6 - DNA, 22 bp, Artificial Sequence
  Synthetic Primer
tacaccttggatgttggacagg

SEQ ID NO: 7 - DNA, 26 bp, Artificial Sequence
  Synthetic Probe
cctcatcctcatcgcggtcggcatca

SEQ ID NO: 8 - DNA, 25 bp, Artificial Sequence
  Synthetic Primer
gctgctccattggttaaatacttcc

SEQ ID NO: 9 - DNA, 22 bp, Artificial Sequence
  Synthetic Primer
gttgtcaactgccatgctgatg

SEQ ID NO: 10 - DNA, 26 bp, Artificial Sequence
  Synthetic Probe
agaacgcagagcacccctggctacat

SEQ ID NO: 11 - DNA, 24 bp, Artificial Sequence
  Synthetic Primer
agtactttgtcggttacctaggag

SEQ ID NO: 12 - DNA, 25 bp, Artificial Sequence
  Synthetic Primer
gtaatagttgaatatgccagcaacg

SEQ ID NO: 13 - DNA, 28 bp, Artificial Sequence
  Synthetic Probe
tcaggctgtccagaatcttccaaccaag

SEQ ID NO: 14 - DNA, 23 bp, Artificial Sequence
  Synthetic Primer
gctcagtgtggagtatctcaaag

SEQ ID NO: 15 - DNA, 21 bp, Artificial Sequence
  Synthetic Primer
cttcagtgtgtccctgtttgc

SEQ ID NO: 16 - DNA, 22 bp, Artificial Sequence
  Synthetic Probe
cgcctggcccagctctccgagg

SEQ ID NO: 17 - DNA, 20 bp, Artificial Sequence
  Synthetic Primer
agatggacgtgagcctcagg

SEQ ID NO: 18 - DNA, 23 bp, Artificial Sequence
  Synthetic Primer
tcagtctggtcgtagatgaaagc

SEQ ID NO: 19 - DNA, 26 bp, Artificial Sequence
  Synthetic Probe
agcgaatgaggcacagcaaccactcc

SEQ ID NO: 20 - DNA, 23 bp, Artificial Sequence
  Synthetic Primer
ccacagcaatttctgggttacag

SEQ ID NO: 21 - DNA, 22 bp, Artificial Sequence
  Synthetic Primer
gacaagcggttcctactcttcc

SEQ ID NO: 22 - DNA, 26 bp, Artificial Sequence
  Synthetic Probe
aacccagcagtcgtctttgtcacccg

SEQ ID NO: 23 - DNA, 25 bp, Artificial Sequence
  Synthetic Primer
tcaaggagtatttctacaccagtgg

SEQ ID NO: 24 - DNA, 22 bp, Artificial Sequence
  Synthetic Primer
tcccgaacccatttcttctctg

SEQ ID NO: 25 - DNA, 26 bp, Artificial Sequence
  Synthetic Probe
taccacagggtcagcctccccgaaac

SEQ ID NO: 26 - DNA, 20 bp, Artificial Sequence
  Synthetic Primer
cagcagtgggcaggaatgac

SEQ ID NO: 27 - DNA, 23 bp, Artificial Sequence
  Synthetic Primer
tcctcatccatctccacagtagg

SEQ ID NO: 28 - DNA, 26 bp, Artificial Sequence
  Synthetic Probe
tgttcccaccgttccctctacccatg

SEQ ID NO: 29 - DNA, 22 bp, Artificial Sequence
  Synthetic Primer
tggtactggctacccttctctc

SEQ ID NO: 30 - DNA, 24 bp, Artificial Sequence
  Synthetic Primer
tccagtcttgtaatgtctgacagc

SEQ ID NO: 31 - DNA, 26 bp, Artificial Sequence
  Synthetic Probe
agggccacctcactttcagcagtctg

SEQ ID NO: 32 - DNA, 21 bp, Artificial Sequence
  Synthetic Primer
aatgcggcagacagttactcc

SEQ ID NO: 33 - DNA, 21 bp, Artificial Sequence
  Synthetic Primer
gtggctggtgactctagtgtc

SEQ ID NO: 34 - DNA, 25 bp, Artificial Sequence
  Synthetic Probe
cgccttcctctacctgctgcactgc

SEQ ID NO: 35 - DNA, 25 bp, Artificial Sequence
  Synthetic Primer
ctatgtgtttaccatcctcaactgc

SEQ ID NO: 36 - DNA, 21 bp, Artificial Sequence
  Synthetic Primer
gcccacttccggtattcttcc

SEQ ID NO: 37 - DNA, 30 bp, Artificial Sequence
  Synthetic Probe
cctctccaccacactgcaaacaatagccac

SEQ ID NO: 38 - DNA, 26 bp, Artificial Sequence
  Synthetic Primer
acatgaatatctccaacaagatcagg

SEQ ID NO: 39 - DNA, 23 bp, Artificial Sequence
  Synthetic Primer
gctgtccttatgccaaatccatc

SEQ ID NO: 40 - DNA, 25 bp, Artificial Sequence
  Synthetic Probe
ccagcgcgatgtcaactgctcggtt

SEQ ID NO: 41 - DNA, 19 bp, Artificial Sequence
  Synthetic Primer
tcttccaggtgggctccag

SEQ ID NO: 42 - DNA, 21 bp, Artificial Sequence
  Synthetic Primer
gccagaattgccaaggtcatc

SEQ ID NO: 43 - DNA, 26 bp, Artificial Sequence
  Synthetic Probe
tgccaacagggtcacagccaggtaca

SEQ ID NO: 44 - DNA, 26 bp, Artificial Sequence
  Synthetic Primer
tgatccttattcagtagcactctctg

SEQ ID NO: 45 - DNA, 21 bp, Artificial Sequence
  Synthetic Primer
agcgtgatgacaactccagtg

SEQ ID NO: 46 - DNA, 24 bp, Artificial Sequence
  Synthetic Probe
cctctccagagacgcagcgacggc

SEQ ID NO: 47 - DNA, 20 bp, Artificial Sequence
  Synthetic Primer
agctgtacgactggccctac

SEQ ID NO: 48 - DNA, 21 bp, Artificial Sequence
  Synthetic Primer
tgccgggtttcgaactcaaag

SEQ ID NO: 49 - DNA, 26 bp, Artificial Sequence
  Synthetic Probe
agcaccaggaaccaggagacttacga

SEQ ID NO: 50 - DNA, 24 bp, Artificial Sequence
  Synthetic Primer
gagaaatcagatggtgtttacacg

SEQ ID NO: 51 - DNA, 22 bp, Artificial Sequence
  Synthetic Primer
ctactgtggtggtttctcatgc

SEQ ID NO: 52 - DNA, 26 bp, Artificial Sequence
  Synthetic Probe
tgtcccagaaacctgtggctgcttca

SEQ ID NO: 53 - DNA, 24 bp, Artificial Sequence
  Synthetic Primer
gatgactatggagacccaaatgtc

SEQ ID NO: 54 - DNA, 22 bp, Artificial Sequence
  Synthetic Primer
caagtttcagcacagcctttgg

SEQ ID NO: 55 - DNA, 26 bp, Artificial Sequence
  Synthetic Probe
cccagtagtttcacctctgttgcccc

SEQ ID NO: 56 - DNA, 23 bp, Artificial Sequence
  Synthetic Primer
tttcttaaatggcccacatcagg

SEQ ID NO: 57 - DNA, 23 bp, Artificial Sequence
  Synthetic Primer
gaaatccagttaagtggctgtcc

SEQ ID NO: 58 - DNA, 26 bp, Artificial Sequence
  Synthetic Probe
agccaaccaccatgaaagcatctcac

SEQ ID NO: 59 - DNA, 20 bp, Artificial Sequence
  Synthetic Primer
accacctccaccatcccatc

SEQ ID NO: 60 - DNA, 26 bp, Artificial Sequence
  Synthetic Primer
acaccatcttgattgtcttcattgac

SEQ ID NO: 61 - DNA, 26 bp, Artificial Sequence
  Synthetic Probe
tggccttactcctcccacagaatgcg

SEQ ID NO: 62 - DNA, 26 bp, Artificial Sequence
  Synthetic Primer
cataccataacaatgacgacaacttc

SEQ ID NO: 63 - DNA, 24 bp, Artificial Sequence
  Synthetic Primer
catgggacaggtagtaacatttgg

SEQ ID NO: 64 - DNA, 26 bp, Artificial Sequence
  Synthetic Probe
agcccagcacagactcatggtagcag

SEQ ID NO: 65 - DNA, 24 bp, Artificial Sequence
  Synthetic Primer
ctttgggtgagttgaacttcttcc

SEQ ID NO: 66 - DNA, 22 bp, Artificial Sequence
  Synthetic Primer
cccagctaactgcaacataacg

SEQ ID NO: 67 - DNA, 26 bp, Artificial Sequence
  Synthetic Probe
accctcgcttcagccacagtttcctc

SEQ ID NO: 68 - DNA, 21 bp, Artificial Sequence
  Synthetic Primer
cccttcctactcccagacacc

SEQ ID NO: 69 - DNA, 24 bp, Artificial Sequence
  Synthetic Primer
agagattttccagtccaacctacc

SEQ ID NO: 70 - DNA, 24 bp, Artificial Sequence
  Synthetic Probe
cctgcttttgctcccagtggctgc

SEQ ID NO: 71 - DNA, 22 bp, Artificial Sequence
  Synthetic Primer
gatccatctgggtcacatcctc

SEQ ID NO: 72 - DNA, 21 bp, Artificial Sequence
  Synthetic Primer
cagggtaaaaggcagggagtg

SEQ ID NO: 73 - DNA, 28 bp, Artificial Sequence
  Synthetic Probe
tgttcccttgtagctgcgtcctttacca

SEQ ID NO: 74 - DNA, 26 bp, Artificial Sequence
  Synthetic Primer
gccatgaatttgacctctatgaaaac

SEQ ID NO: 75 - DNA, 24 bp, Artificial Sequence
  Synthetic Primer
gctgacatttgatgccactcttag

SEQ ID NO: 76 - DNA, 26 bp, Artificial Sequence
  Synthetic Probe
ccttcgtgagcacagcatagggacct

SEQ ID NO: 77 - DNA, 23 bp, Artificial Sequence
  Synthetic Primer
cagttcagcatcagttccaagac

SEQ ID NO: 78 - DNA, 19 bp, Artificial Sequence
  Synthetic Primer
ttgtacgtctgcggcatgg

SEQ ID NO: 79 - DNA, 23 bp, Artificial Sequence
  Synthetic Probe
tcgcccacggagccctcagacaa

SEQ ID NO: 80 - DNA, 20 bp, Artificial Sequence
  Synthetic Primer
aaactgttccgcagccagag

SEQ ID NO: 81 - DNA, 20 bp, Artificial Sequence
  Synthetic Primer
aactcggcctcccactgtac

SEQ ID NO: 82 - DNA, 26 bp, Artificial Sequence
  Synthetic Probe
tcagccagtcccttagacacaccact

SEQ ID NO: 83 - DNA, 26 bp, Artificial Sequence
  Synthetic Primer
cctagaggatcttgttactgaatacc

SEQ ID NO: 84 - DNA, 22 bp, Artificial Sequence
  Synthetic Primer
agagtcgttcactgtagtctgg

SEQ ID NO: 85 - DNA, 22 bp, Artificial Sequence
  Synthetic Probe
ttgcccgcctcccagaccacca

SEQ ID NO: 86 - DNA, 21 bp, Artificial Sequence
  Synthetic Primer
cccgcatccctcacatgaaag

SEQ ID NO: 87 - DNA, 21 bp, Artificial Sequence
  Synthetic Primer
agtcaccagacactcctccag

SEQ ID NO: 88 - DNA, 25 bp, Artificial Sequence
  Synthetic Probe
tctgatccacctgagcgacctccgg

SEQ ID NO: 89 - DNA, 21 bp, Artificial Sequence
  Synthetic Primer
tcctgctggtcatctggaagg

SEQ ID NO: 90 - DNA, 22 bp, Artificial Sequence
  Synthetic Primer
cagcaaacttggggttcatgac

SEQ ID NO: 91 - DNA, 29 bp, Artificial Sequence
  Synthetic Probe
ttcgtatccatctggcagcttgacggtca

SEQ ID NO: 92 - DNA, 18 bp, Artificial Sequence
  Synthetic Primer
gcgggaggctgtctttcc

SEQ ID NO: 93 - DNA, 18 bp, Artificial Sequence
  Synthetic Primer
caggttgaggcggttggg

SEQ ID NO: 94 - DNA, 26 bp, Artificial Sequence
  Synthetic Probe
tgccagcatctgctctcactgcaacc

SEQ ID NO: 95 - DNA, 20 bp, Artificial Sequence
  Synthetic Primer
tgaagcccaagagccaaagg

SEQ ID NO: 96 - DNA, 21 bp, Artificial Sequence
  Synthetic Primer
tcctgcttgtctggtaagtgc

SEQ ID NO: 97 - DNA, 25 bp, Artificial Sequence
  Synthetic Probe
agaccttgccacctgacctcctgag

SEQ ID NO: 98 - DNA, 20 bp, Artificial Sequence
  Synthetic Primer
ccttgaccttggggagaacc

SEQ ID NO: 99 - DNA, 25 bp, Artificial Sequence
  Synthetic Primer
ttctagatgtagccgttctaattgc

SEQ ID NO: 100 - DNA, 26 bp, Artificial Sequence
  Synthetic Probe
ctatcccatctgtgaggcggctctgc

SEQ ID NO: 101 - DNA, 24 bp, Artificial Sequence
  Synthetic Primer
atgtctcaaggctcatctgttttg

SEQ ID NO: 102 - DNA, 23 bp, Artificial Sequence
  Synthetic Primer
tgacaggcccagcatagtaaatc

SEQ ID NO: 103 - DNA, 27 bp, Artificial Sequence
  Synthetic Probe
ttggctcctggaccatattctctgggt

SEQ ID NO: 104 - DNA, 27 bp, Artificial Sequence
  Synthetic Primer
atattcatgttttacaaggagaaaccc

SEQ ID NO: 105 - DNA, 26 bp, Artificial Sequence
  Synthetic Primer
agatttgctttctgtctatcacaatg

SEQ ID NO: 106 - DNA, 24 bp, Artificial Sequence
  Synthetic Probe
cgttgaccgcatggcagctccgag

SEQ ID NO: 107 - DNA, 18 bp, Artificial Sequence
  Synthetic Primer
agagcgtgtccccacagg

SEQ ID NO: 108 - DNA, 23 bp, Artificial Sequence
  Synthetic Primer
cgactgaggaggaagatcacatc

SEQ ID NO: 109 - DNA, 26 bp, Artificial Sequence
  Synthetic Probe
actggtcagcatgtgcatctgccctg

SEQ ID NO: 110 - DNA, 21 bp, Artificial Sequence
  Synthetic Primer
ggggagcactactccgagaag

SEQ ID NO: 111 - DNA, 22 bp, Artificial Sequence
  Synthetic Primer
cacgtatccgatgtcaggtctc

SEQ ID NO: 112 - DNA, 30 bp, Artificial Sequence
  Synthetic Probe
acgcttaccatcgtaaaggcacgtccaatt

SEQ ID NO: 113 - DNA, 26 bp, Artificial Sequence
  Synthetic Primer
agaggttataatctgaatcccaaagg

SEQ ID NO: 114 - DNA, 26 bp, Artificial Sequence
  Synthetic Primer
taaaggagtcattatagaagcagtgg

SEQ ID NO: 115 - DNA, 26 bp, Artificial Sequence
  Synthetic Probe
tccacccaaggctctgcgacttccat

SEQ ID NO: 116 - DNA, 21 bp, Artificial Sequence
  Synthetic Primer
gactacagcaacgaggagcac

SEQ ID NO: 117 - DNA, 25 bp, Artificial Sequence
  Synthetic Primer
ggtcgctctgcataaaatattcctc

SEQ ID NO: 118 - DNA, 24 bp, Artificial Sequence
  Synthetic Probe
tcgtgttgagacctcccgcgcagt

SEQ ID NO: 119 - DNA, 24 bp, Artificial Sequence
  Synthetic Primer
acatgtattcaactgcaccaagac

SEQ ID NO: 120 - DNA, 22 bp, Artificial Sequence
  Synthetic Primer
tgggcaatccataaccaaaacc

SEQ ID NO: 121 - DNA, 27 bp, Artificial Sequence
  Synthetic Probe
ttgccgtaggtcccactgttgtcttgc

SEQ ID NO: 122 - DNA, 22 bp, Artificial Sequence
  Synthetic Primer
gatcagactcagcctcctcaga

SEQ ID NO: 123 - DNA, 18 bp, Artificial Sequence
  Synthetic Primer
gctgctgccctcccagat

SEQ ID NO: 124 - DNA, 26 bp, Artificial Sequence
  Synthetic Probe
agacccctcctggccgctactctttt

SEQ ID NO: 125 - DNA, 19 bp, Artificial Sequence
  Synthetic Primer
tggcccagagtggcatgtc

SEQ ID NO: 126 - DNA, 20 bp, Artificial Sequence
  Synthetic Primer
ggctcaggtaggctcagacc

SEQ ID NO: 127 - DNA, 26 bp, Artificial Sequence
  Synthetic Probe
ccagcacctcgcatcagccagagttg

SEQ ID NO: 128 - DNA, 22 bp, Artificial Sequence
  Synthetic Primer
gggtttccagcagcatttgttg

SEQ ID NO: 129 - DNA, 24 bp, Artificial Sequence
  Synthetic Primer
ccacttgatgtctccagcactaag

SEQ ID NO: 130 - DNA, 26 bp, Artificial Sequence
  Synthetic Probe
acatctccatctgggacaccgcaggg

SEQ ID NO: 131 - DNA, 21 bp, Artificial Sequence
  Synthetic Primer
gccttctacctgaagcagtgg

SEQ ID NO: 132 - DNA, 22 bp, Artificial Sequence
  Synthetic Primer
tgccggtgattcacatcatagg

SEQ ID NO: 133 - DNA, 26 bp, Artificial Sequence
  Synthetic Probe
ttttgactccctggttgctcccctgg

SEQ ID NO: 134 - DNA, 23 bp, Artificial Sequence
  Synthetic Primer
gccaacatcaatgtggaaaatgc

SEQ ID NO: 135 - DNA, 20 bp, Artificial Sequence
  Synthetic Primer
ctgctcctcttctgctggtc

SEQ ID NO: 136 - DNA, 23 bp, Artificial Sequence
  Synthetic Probe
cgccgtcaggattgctgccgtca

SEQ ID NO: 137 - DNA, 20 bp, Artificial Sequence
  Synthetic Primer
agggaccgctctcttctctc

SEQ ID NO: 138 - DNA, 20 bp, Artificial Sequence
  Synthetic Primer
cctcaaccagccagtgttcc

SEQ ID NO: 139 - DNA, 25 bp, Artificial Sequence
  Synthetic Probe
agtctccgcgctgtagctcctgtga

SEQ ID NO: 140 - DNA, 20 bp, Artificial Sequence
  Synthetic Primer
ccctgaaccccagaacaacc

SEQ ID NO: 141 - DNA, 23 bp, Artificial Sequence
  Synthetic Primer
gggaagtgaacagttttggaacc

SEQ ID NO: 142 - DNA, 26 bp, Artificial Sequence
  Synthetic Probe
ccttgtcggctgtggtgtctctgctc

SEQ ID NO: 143 - DNA, 21 bp, Artificial Sequence
  Synthetic Primer
aagctctttgggctggtgatg

SEQ ID NO: 144 - DNA, 24 bp, Artificial Sequence
  Synthetic Primer
ggtggaagaatgtcagaagaatgg

SEQ ID NO: 145 - DNA, 28 bp, Artificial Sequence
  Synthetic Probe
aaggactcccacaacgaactcaatccca

SEQ ID NO: 146 - DNA, 21 bp, Artificial Sequence
  Synthetic Primer
tacgacatgctggggatcatg

SEQ ID NO: 147 - DNA, 26 bp, Artificial Sequence
  Synthetic Primer
gtagccgtaaacaacaatggtatttc

SEQ ID NO: 148 - DNA, 26 bp, Artificial Sequence
  Synthetic Probe
tggtccgtccacaagcaatgagtgcc

SEQ ID NO: 149 - DNA, 21 bp, Artificial Sequence
  Synthetic Primer
actgttggctgtgaggaatgc

SEQ ID NO: 150 - DNA, 22 bp, Artificial Sequence
  Synthetic Primer
ccttttcagagccttggaggag

SEQ ID NO: 151 - DNA, 25 bp, Artificial Sequence
  Synthetic Probe
cccgagtgacaagcctgtagcccat

SEQ ID NO: 152 - DNA, 23 bp, Artificial Sequence
  Synthetic Primer
ccaggcagtcagatcatcttctc

SEQ ID NO: 153 - DNA, 20 bp, Artificial Sequence
  Synthetic Primer
ctctcagctccacgccattg

SEQ ID NO: 154 - DNA, 24 bp, Artificial Sequence
  Synthetic Probe
ccttggctgggaagcacaccctgc

SEQ ID NO: 155 - DNA, 18 bp, Artificial Sequence
  Synthetic Primer
cctgcaagccctggacca

SEQ ID NO: 156 - DNA, 21 bp, Artificial Sequence
  Synthetic Primer
agattgcgtccgagctattgc

SEQ ID NO: 157 - DNA, 25 bp, Artificial Sequence
  Synthetic Probe
tcttctggaccctgaacggcacgct

SEQ ID NO: 158 - DNA, 19 bp, Artificial Sequence
  Synthetic Primer
accagctccaggagaaggc

SEQ ID NO: 159 - DNA, 22 bp, Artificial Sequence
  Synthetic Primer
cagttgcaagcagtcttgagtg

SEQ ID NO: 160 - DNA, 25 bp, Artificial Sequence
  Synthetic Probe
ctccttactcgtccacgccgcctca

SEQ ID NO: 161 - DNA, 20 bp, Artificial Sequence
  Synthetic Primer
tccgagaagtggttgctgac

SEQ ID NO: 162 - DNA, 26 bp, Artificial Sequence
  Synthetic Primer
tcttgaaataacccaaatgtgaatcc

SEQ ID NO: 163 - DNA, 26 bp, Artificial Sequence
  Synthetic Probe
cgctgtagacatccgacctctgaccc

SEQ ID NO: 164 - DNA, 21 bp, Artificial Sequence
  Synthetic Primer
ctgagaccgagtcgccttatc

SEQ ID NO: 165 - DNA, 21 bp, Artificial Sequence
  Synthetic Primer
atacggcctctgtgtgttgag

SEQ ID NO: 166 - DNA, 27 bp, Artificial Sequence
  Synthetic Probe
agtaccgataacagccatgcactgtgc

SEQ ID NO: 167 - DNA, 24 bp, Artificial Sequence
  Synthetic Primer
agtgctgtgatgatggacaattac

SEQ ID NO: 168 - DNA, 20 bp, Artificial Sequence
  Synthetic Primer
ttgctctcctgtgcagaagg

SEQ ID NO: 169 - DNA, 26 bp, Artificial Sequence
  Synthetic Probe
tctcaccagtgtgcttcctgctgtgc

SEQ ID NO: 170 - DNA, 20 bp, Artificial Sequence
  Synthetic Primer
gtcggcatctgagaccagtg

SEQ ID NO: 171 - DNA, 23 bp, Artificial Sequence
  Synthetic Primer
gttcacagtccttgaagtcacac

SEQ ID NO: 172 - DNA, 26 bp, Artificial Sequence
  Synthetic Probe
ccactggttctggactgctctgcgtt

SEQ ID NO: 173 - DNA, 21 bp, Artificial Sequence
  Synthetic Primer
cccagaggaaacggacacaac

SEQ ID NO: 174 - DNA, 23 bp, Artificial Sequence
  Synthetic Primer
tgcagatgcttctcctgttgtag

SEQ ID NO: 175 - DNA, 140 bp, Artificial Sequence
  PCR product
gtgctttctggagggtctactatgtatcctggcctgccatcacggttggaacgagaacttaaacagctttacttagaacgagttttgaagggtgatgtggaaaaactttctaaatttaagatccgcattgaagacccacc

SEQ ID NO: 176 - DNA, 150 bp, Artificial Sequence
  PCR product
gtagagtggaaaaggagcattggatatgatgatactgatgagtcccactgtgctgagcacattgagtcacgtactcttgcaattgcccgcaacctgactcagcagctccagaccacgtgccacaccctcctgtccaacatccaaggtgta

SEQ ID NO: 177 - DNA, 108 bp, Artificial Sequence
  PCR product
gctgctccattggttaaatacttccaggaaaaggggctcatcatgacatttgatgccgaccgcgatgaggatgaggtgttctatgacatcagcatggcagttgacaac

SEQ ID NO: 178 - DNA, 116 bp, Artificial Sequence
  PCR product
agtactttgtcggttacctaggagagagaacgcagagcacccctggctacatatttgggaaacgcatcatactcttcctgttcctcatgtccgttgctggcatattcaactattac

SEQ ID NO: 179 - DNA, 124 bp, Artificial Sequence
  PCR product
gctcagtgtggagtatctcaaagcctttcaggctgtccagaatcttccaaccaagaagcagcaactacaggctttgaaccttcttgtcatcctcctacctgatgcaaacagggacacactgaag

SEQ ID NO: 180 - DNA, 103 bp, Artificial Sequence
  PCR product
tcggggacgagatggacgtgagcctcagggccccgcgcctggcccagctctccgaggtggccatgcacagcctgggtctggctttcatctacgaccagactga

SEQ ID NO: 181 - DNA, 145 bp, Artificial Sequence
  PCR product
ccacagcaatttctgggttacagagtcagcgaatgaggcacagcaaccactccacagagaactctccaattgaaagacgaagtctaatgacctctgatgaaaattatcacaatgaaagggctcggaagagtaggaaccgcttgtc

SEQ ID NO: 182 - DNA, 109 bp, Artificial Sequence
  PCR product
tcaaggagtatttctacaccagtggcaagtgctccaacccagcagtcgtctttgtcacccgaaagaaccgccaagtgtgtgccaacccagagaagaaatgggttcggga

SEQ ID NO: 183 - DNA, 136 bp, Artificial Sequence
  PCR product
cagcagtgggcaggaatgacacccaccctaccacagggtcagcctccccgaaacaccagaagaagtccaagttacatggccccactgaaacctcaagctgttcaggtgccgcccctactgtggagatggatgagga

SEQ ID NO: 184 - DNA, 148 bp, Artificial Sequence
  PCR product
tggtactggctacccttctctcgcaagtgagccccttcaagatacctatagaggaacttgaggacagagtgtttgtgaattgcaataccagcatcacatgggtagagggaacggtgggaacactgctctcagacattacaagactgga

SEQ ID NO: 185 - DNA, 132 bp, Artificial Sequence
  PCR product
aatgcggcagacagttactcctgggttccagagcgagctgagagcagggccatggagaaccagtacagtccgacacctgggacagactgctgaaagtgaggtggccctagagacactagagtcaccagccac

SEQ ID NO: 186 - DNA, 99 bp, Artificial Sequence
  PCR product
ctatgtgtttaccatcctcaactgcctgcagggcgccttcctctacctgctgcactgcctgctcaacaagaaggttcgggaagaataccggaagtgggc

SEQ ID NO: 187 - DNA, 143 bp, Artificial Sequence
  PCR product
acatgaatatctccaacaagatcaggaacctctccaccacactgcaaacaatagccaccaaattatgtcgtgagctatatagcaaagaacaagagcacaaatgtaagccttgtccaaggagatggatttggcataaggacagc

SEQ ID NO: 188 - DNA, 128 bp, Artificial Sequence
  PCR product
tcttccaggtgggctccagcggttcccagcgcgatgtcaactgctcggttatgggaccacaagaaaaaaaagtagtggtgtaccttcagaagctggatacagcatatgatgaccttggcaattctggc

SEQ ID NO: 189 - DNA, 150 bp, Artificial Sequence
  PCR product
tgatccttattcagtagcactctctgaacttggagacaggcaaaatgaaagttatctcaattttgctcgaaagagaataaagaaccctgaaggaggcctgtacctggctgtgaccctgttggcaggcatcactggagttgtcatcacgct

SEQ ID NO: 190 - DNA, 121 bp, Artificial Sequence
  PCR product
agctgtacgactggccctacaggtttctgcggcgctttgggcgggacaaggtaaccttttcctttgaggcaggccgtcgctgcgtctctggagagggcaactttgagttcgaaacccggca

SEQ ID NO: 191 - DNA, 87 bp, Artificial Sequence
  PCR product
gagaaatcagatggtgtttacacgggcctgagcaccaggaaccaggagacttacgagactctgaagcatgagaaaccaccacagtag

SEQ ID NO: 192 - DNA, 137 bp, Artificial Sequence
  PCR product
gatgactatggagacccaaatgtctcagaatgtatgtcccagaaacctgtggctgcttcaaccattgacagttttgctgctgctggcttctgcagacagtcaagctgcagctcccccaaaggctgtgctgaaacttg

SEQ ID NO: 193 - DNA, 110 bp, Artificial Sequence
  PCR product
tttcttaaatggcccacatcaggacagctgttccttggccatcccaaagctattctgggggcaacagaggtgaaactactgggccatggacagccacttaactggatttc

SEQ ID NO: 194 - DNA, 147 bp, Artificial Sequence
  PCR product
accacctccaccatcccatccggccagccaaccaccattgccagcatctcacccatcacaaccaccagtcccaagcctacctcccagaaacattaaacctccgtttgacctaaaaagccctgtcaatgaagacaatcaagatggtgt

SEQ ID NO: 195 - DNA, 135 bp, Artificial Sequence
  PCR product
cataccataacaatgacgacaacttcagtcagcagctggccttactcctcccacagaatgcgctttataaccaatcatagcgaccaaccgccacaaaacttctcagcaacaccaaatgttactacctgtcccatg

SEQ ID NO: 196 - DNA, 144 bp, Artificial Sequence
  PCR product
ctttgggtgagttgaacttcttccattatagaaagaattgaaggctgagaaactcagcctctatcatgtggaacagctctgacgccaacttctcctgctaccatgagtctgtgctgggctatcgttatgttgcagttagctggg

SEQ ID NO: 197 - DNA, 84 bp, Artificial Sequence
  PCR product
cccttcctactcccagacacccaccctcgcttcagccacagtttcctcatctgtccagtgggtaggttggactggaaaatctct

SEQ ID NO: 198 - DNA, 98 bp, Artificial Sequence
  PCR product
gatccatctgggtcacatcctcttcctgcttttgctcccagtggctgcagctcagacgactccaggagagagatcatcactccctgccttttaccctg

SEQ ID NO: 199 - DNA, 114 bp, Artificial Sequence
  PCR product
gccatgaatttgacctctatgaaaacaaagactacattagaaactgcatcattggtaaaggacgcagctacaagggaacagtatctatcactaagagtggcatcaaatgtcagc

SEQ ID NO: 200 - DNA, 79 bp, Artificial Sequence
  PCR product
cagttcagcatcagttccaagacacgtttccaggtccctatgctgtgctcacgaaggacaccatgccgcagacgtacaa

SEQ ID NO: 201 - DNA, 110 bp, Artificial Sequence
  PCR product
aaactgttccgcagccagagcgctgatgcccccggccccacagagcgcgagcggctaaagaagatgttgtctgagggctccgtgggcgaggtacagtgggaggccgagtt

SEQ ID NO: 202 - DNA, 104 bp, Artificial Sequence
  PCR product
cctagaggatcttgttactgaataccacgggaacttttcggcctggagtggtgtgtctaagggactggctgagagtctgcagccagactacagtgaacgactct

SEQ ID NO: 203 - DNA, 104 bp, Artificial Sequence
  PCR product
cccgcatccctcacatgaaagaccccatcggtgacagcttccaaaacgacaagctggtggtctgggaggcgggcaaagccggcctggaggagtgtctggtgact

SEQ ID NO: 204 - DNA, 150 bp, Artificial Sequence
  PCR product
tcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctg

SEQ ID NO: 205 - DNA, 127 bp, Artificial Sequence
  PCR product
gcgggaggctgtctttcccttccagcctggaagtgttgcagaggtgtgcatcaccttcgaccaggccaacctgaccgtcaagctgccagatggatacgaattcaagttccccaaccgcctcaacctg

SEQ ID NO: 206 - DNA, 150 bp, Artificial Sequence
  PCR product
tgaagcccaagagccaaaggaatcaccaccaccttctaaaacgtcagcagctgctcagttggatgagctcatggctcacctgactgagatgcaggccaaggttgcagtgagagcagatgctggcaagaagcacttaccagacaagcagga

SEQ ID NO: 207 - DNA, 85 bp, Artificial Sequence
  PCR product
ccttgaccttggggagaaccagttggagaccttgccacctgacctcctgaggggtccgctgcaattagaacggctacatctagaa

SEQ ID NO: 208 - DNA, 118 bp, Artificial Sequence
  PCR product
atgtctcaaggctcatctgttttgaatttctcctatcccatctgtgaggcggctctgcccaagttttctttctgtggaagaaggaaaggagagcagatttactatgctgggcctgtca

SEQ ID NO: 209 - DNA, 116 bp, Artificial Sequence
  PCR product
atattcatgttttacaaggagaaacccattgattggctcctggaccatattctctgggtgaaagtctgcaaccctgaaaaagatgcaaaacattgtgatagacagaaagcaaatct

SEQ ID NO: 210 - DNA, 133 bp, Artificial Sequence
  PCR product
agagcgtgtccccacagggcaacagcgttgaccgcatggcagctccgagagcagaggctctatttgacttcactggaaacagcaaactggagctgaatttcaaagctggagatgtgatcttcctcctcagtcg

SEQ ID NO: 211 - DNA, 87 bp, Artificial Sequence
  PCR product
ggggagcactactccgagaagttacgagaactggtcagcatgtgcatctgccctgacccccaccagagacctgacatcggatacgtg

SEQ ID NO: 212 - DNA, 125 bp, Artificial Sequence
  PCR product
agaggttataatctgaatcccaaaggagactgcagctgatgaaagtgcttccaaactgaaaattggacgtgcctttacgatggtaagcgttaacagctcccactgcttctataatgactccttta

SEQ ID NO: 213 - DNA, 142 bp, Artificial Sequence
  PCR product
gactacagcaacgaggagcacatgaccctgctgaagatgattttgataaaatgctgtgatatctctaacgaggtccgtccaatggaagtcgcagagccttgggtggactgtttattagaggaatattttatgcagagcgacc

SEQ ID NO: 214 - DNA, 81 bp, Artificial Sequence
  PCR product
acatgtattcaactgcaccaagacctcgtgttgagacctcccgcgcagtgcctctggctggttttggttatggattgccca

SEQ ID NO: 215 - DNA, 125 bp, Artificial Sequence
  PCR product
gatcagactcagcctcctcagagcctgttgggatatatcagggtttcgagaagaagaccggagttgctggggaggacatgcaagacaacagtgggacctacggcaagatctgggagggcagcagc

SEQ ID NO: 216 - DNA, 105 bp, Artificial Sequence
  PCR product
tggcccagagtggcatgtcccagccagacccctcctggccgctactctttttccctcttctctgggctctcccactcctggggccggtctgagcctacctgagcc

SEQ ID NO: 217 - DNA, 96 bp, Artificial Sequence
  PCR product
gggtttccagcagcatttgttgcagcatgggctgtggcacgagcaactctggctgatgcgaggtgctgggaacttagtgctggagacatcaagtgg

SEQ ID NO: 218 - DNA, 140 bp, Artificial Sequence
  PCR product
gccttctacctgaagcagtggcgctcctacaacatctccatctgggacaccgcagggcgggagcagttccacggcctgggctccatgtactgccggggggcggccgccatcatcctcacctatgatgtgaatcaccggca

SEQ ID NO: 219 - DNA, 140 bp, Artificial Sequence
  PCR product
gccaacatcaatgtggaaaatgcatttttcactctcgccagagatatcaaagcaaaaatggacaaaaaattggaaggcaacagcccccaggggagcaaccagggagtcaaaatcacaccggaccagcagaagaggagcag

SEQ ID NO: 220 - DNA, 119 bp, Artificial Sequence
  PCR product
agggaccgctctcttctctcgccgacagcttttccttccctccatgagaaagaagccagctctgtctgacggcagcaatcctgacggcgatctcctccaggaacactggctggttgagg

SEQ ID NO: 221 - DNA, 91 bp, Artificial Sequence
  PCR product
ccctgaaccccagaacaaccagctggatcagttctcacaggagctacagcgcggagactgggaaacatggttccaaaactgttcacttccc

SEQ ID NO: 222 - DNA, 148 bp, Artificial Sequence
  PCR product
aagctctttgggctggtgatggccttgtcggctgtggtgtctctgctccagttccccatcttcaccctcatcaaaggctcccttcagaatgacccattttacgtgaatgtgatgttcatgcttgccattcttctgacattcttccacc

SEQ ID NO: 223 - DNA, 150 bp, Artificial Sequence
  PCR product
tacgacatgctggggatcatggcatggaatgcaacttgcaaaaactggctggcagcagaggctgccctggaaaagtactacctttccattttttatgggattgagttcgttgtgggagtccttggaaataccattgttgtttacggctac

SEQ ID NO: 224 - DNA, 112 bp, Artificial Sequence
  PCR product
actgttggctgtgaggaatgcacagtgtttccctgtttatccatcccctgcaaactgcagagtggcactcattgcttgtggacggaccagctcctccaaggctctgaaaagg

SEQ ID NO: 225 - DNA, 140 bp, Artificial Sequence
  PCR product
ccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagag

SEQ ID NO: 226 - DNA, 78 bp, Artificial Sequence
  PCR product
cctgcaagccctggaccaactgcaccttggctgggaagcacaccctgcagccggccagcaatagctcggacgcaatct

SEQ ID NO: 227 - DNA, 97 bp, Artificial Sequence
  PCR product
accagctccaggagaaggcaactccagtcagaacagcagaaataagcgtgccgttcagggtccagaagaaacagtcactcaagactgcttgcaactg

SEQ ID NO: 228 - DNA, 100 bp, Artificial Sequence
  PCR product
tccgagaagtggttgctgacagccacaaagtgaaagggagtgaggcggcgtggacgagtaaggagtgacagtgaggattcacatttgggttatttcaaga

SEQ ID NO: 229 - DNA, 80 bp, Artificial Sequence
  PCR product
ctgagaccgagtcgccttatcaggagctccagggtcagaggtcggatgtctacagcgacctcaacacacagaggccgtat

SEQ ID NO: 230 - DNA, 82 bp, Artificial Sequence
  PCR product
agtgctgtgatgatggacaattacatagtaccgataacagccatgcactgtgcaaagcatgcccttctgcacaggagagcaa

SEQ ID NO: 231 - DNA, 149 bp, Artificial Sequence
  PCR product
gtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaac

SEQ ID NO: 232 - DNA, 124 bp, Artificial Sequence
  PCR product
cccagaggaaacggacacaactccgggaagcctttgagcagctccaggccaagaaacaaatggccatggagaaacgcagagcagtccagaaccagtggcagctacaacaggagaagcatctgca

(19) United States
(12) Patent Application Publication
(10) Pub. No.: US 2012/0070450 A1
(43) Pub. Date: Mar. 22, 2012

(54) LEUKEMIA STEM CELL MARKERS

(75) Inventors: Fumihiko Ishikawa, Kanagawa (JP); Osamu Ohara, Kanagawa (JP); Yoriko Saito, Kanagawa (JP); Hiroshi Kitamura, Kanagawa (JP); Atsushi Hijikata, Kanagawa (JP); Hidetoshi Ozawa, Kanagawa (JP); Leonard D. Shultz, Bar Harbor, ME (US)

(73) Assignee: RIKEN, Wako-shi (JP)

(21) Appl. No.: 13/258,993

(22) PCT Filed: Mar. 24, 2010
(86) PCT No.: PCT/JP2010/055131

§ 371 Date: Dec. 7, 2011

(30) Foreign Application Priority Data
Mar. 24, 2009 (JP) 2009-072400

(51) Int. Cl. A61K 39/395; C12Q 1/68; G01N 21/64; A61P 35/02; C40B 30/04; A61K 31/7088; C07K 16/18; C07H 21/00; A61K 35/12; C12N 5/078

(52) U.S. Cl. 424/173.1; 424/178.1; 424/93.7; 435/6.14; 435/7.23; 435/375; 506/9; 514/44 A; 530/389.6; 530/391.7; 536/24.5

(57) ABSTRACT

The invention provides a test method for predicting the initial onset or a recurrence of acute myeloid leukemia (AML) comprising (1) measuring the expression level of human leukemic stem cell (LSC) marker genes in a biological sample collected from a subject for a transcription product or translation product of the gene as an analyte and (2) comparing the expression level with a reference value; an LSC-targeting therapeutic agent for AML capable of suppressing the expression of a gene selected from among LSC marker genes or a substance capable of suppressing the activity of a translation product of the gene; a method for producing a sample containing hematopoietic cells for autologous transplantation or allogeneic transplantation for AML patients comprising obtaining an LSC-purged sample with at least 1 kind of LSC marker as an index; and a method of preventing or treating AML.

### TECHNICAL FIELD

The present invention relates to leukemic stem cell markers and the field of treatment of acute myeloid leukemia.

### BACKGROUND ART

Acute myeloid leukemia (AML) is the most common/highly frequent (onset rate) adult leukemia, characterized by the clonal expansion of immature myeloblasts initiating from rare leukemic stem cells (LSCs) (non-patent documents 1-3). The functional and molecular characteristics of human LSCs are largely undetermined. Although conventional chemotherapeutic agents can temporarily remit AML, recurrence later is the difficult problem that prevents us from helping patients. For the development of an effective therapeutic agent or treatment method, elucidation of the recurrence mechanism by clarifying the leukemia features unknown to date is strongly desired.

A recent study demonstrated that a certain ratio of leukemias and cancers consists of a heterogenous cell fraction and is not configured with a homogenous cell population capable of clonal proliferation. Lapidot and Dick identified such heterogeneity in acute myeloid leukemia (AML) and reported that CD34+CD38− cells are transplanted selectively in CB17-scid and NOD/SCID mice (Non-patent Document 4).

The present inventors have succeeded in the development of an animal model capable of reproducing features of human, rather than mouse, AML, particularly AML of individual patients, rather than a cell line, and permitting long-term assessment (Non-patent Document 5, Patent Application PCT/JP2008/068892). The present inventors further identified using a neonatal NOD/SCID/IL2rg KO mouse model, which is one of the most sensitive human stem cell assays, that CD34+CD38-AML cells meet all criteria for cancer stem cells recommended by the American Association for Cancer Research (Non-patent Document 6). Specifically, CD34+CD38− AML cells self-renew, produce non-stem leukemia cells, and have the exclusive capability of causing AML in living organisms. By repeating primary human AML in NOD/SCID/IL2rg KO mice, the present inventors searched for the mechanism behind the chemotherapy resistance and recurrences, which pose the most important problem in the reality of this disease, and identified the following two essential features of human AML stem cells. First, AML stem cells are present predominantly in the endosteal region of the bone marrow; when human AML transplantation recipient mice were treated with chemotherapeutic agents, the great majority of chemotherapy-resistant AML cells were found in osteoblast niches. Second, AML stem cells (not CD34+CD38+and CD34-AML cells) are stationary and hence exhibit resistance to cell cycle-dependent chemotherapeutic agents. These histological experiments and cell cycle analyses agree with the clinical evidence that a large number of AML patients achieve remission via chemotherapy induction but eventually experience recurrences. To develop a novel therapeutic strategy designed to exterminate LSCs seems to be an exact step toward overcoming recurrences of AML.

### Prior Art References

### [Non-Patent Documents]

non-patent document 1: Passegue, E., Jamieson, C. H., Ailles, L. E. & Weissman, I. L. Normal and leukemic hematopoiesis: are leukemias a stem cell disorder or a reacquisition of stem cell characteristics? Proc Natl Acad Sci USA 100 Suppl 1, 11842-11849 (2003).
non-patent document 2: Hope, K. J., Jin, L. & Dick, J. E. Acute myeloid leukemia originates from a hierarchy of leukemic stem cell classes that differ in self-renewal capacity. Nat Immunol 5, 738-743 (2004).
non-patent document 3: Jordan, C. T. & Guzman, M. L. Mechanisms controlling pathogenesis and survival of leukemic stem cells. Oncogene 23, 7178-7187 (2004).
non-patent document 4: Lapidot, T. et al. A cell initiating human acute myeloid leukaemia after transplantation into SCID mice. Nature 367, 645-648 (1994).
non-patent document 5: Ishikawa, F. et al. Chemotherapy-resistant human AML stem cells home to and engraft within the bone marrow endosteal region. Nature Biotechnol 25:1315-1321 (2007).
non-patent document 6: Clarke, M. F. et al. Cancer stem cells—perspectives on current status and future directions: AACR Workshop on cancer stem cells. Cancer Res 66, 9339-9344 (2006).

### SUMMARY OF THE INVENTION

### Problems to Be Solved by the Invention

A problem to be solved is to find a molecular target that is specific for human leukemic stem cells (LSCs) and provide a therapeutic means that will lead to radical treatment of acute myeloid leukemia (AML) and the like.

### Means of Solving the Problems

The present inventors found sets of genes differentially expressed between LSCs and non-stem cells, and proposed the possibility that these genes serve as therapeutic targets for AML (Ishikawa F. et al., Nature Biotechnol 25:1315-1321, 2007 and PCT/JP2008/068892), but were unable to rule out the possibility that the genes are at the same time differentially expressed in normal hematopoietic stem cells (HSCs) as well. Hence, a therapeutic agent and therapeutic method for AML with low prevalence of adverse reactions cannot be realized unless not only a comparison is made between LSCs and non-stem cells, but also a set of genes that are differentially expressed between LSCs and HSCs are identified as targets. The present inventors succeeded in developing a mouse model enabling reproduction of human AML (mice generated by transplanting a fraction containing leukemic stem cells derived from a human AML patient to NOD/SCID/IL2rg^{null} mice), transplanting a small number of bone marrow cells derived from an AML patient, and reconstructing the pathology of AML in the animal model. The present inventors then prepared LSCs derived from an AML patient and those from an AML transplantation recipient mouse, as well as bone marrow samples and cord blood samples (HSCs are contained) derived from healthy donors, conducted a comprehensive analysis, and have developed the present invention.

Accordingly, the present invention provides the following.

[1] A test method for predicting the initial onset or a recurrence of acute myeloid leukemia, comprising
(1) a step of measuring the expression level of leukemic stem cell marker genes in a biological sample collected from a subject for a transcription product or translation product of the genes as an analyte, and
(2) a step of comparing the expression levels obtained in the measuring step with a reference value;

wherein the leukemic stem cell marker genes are 2-218 genes selected from the group consisting of:

cell membrane- or extracellularly-localized genes consisting of ADFP, ALOX5AP, AZU1, C3AR1, CACNB4, CALCRL, CCL4, CCL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, IL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT; apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C17orf60, C18orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, S100P, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT1, WSB2 and ZNF253;

and wherein when the expression of two or more leukemic stem cell marker genes in the subject is significantly higher than the reference value, a possible presence of a leukemic stem cell in the collected biological sample or the subject's body is suggested.

[2] The test method according to [1], wherein the leukemic stem cell marker genes are 2-58 genes selected from the group consisting of:
cell membrane- or extracellularly-localized genes consisting of ADFP, ALOX5AP, CACNB4, CCL5, CD33, CD3D, CD93, CD97, CLEC12A, DOK2, FCER1G, FCGR2A, FUCA2, GPR34, GPR84, HCST, HGF, HOMER3, IL2RA, IL2RG, IL3RA, ITGB2, LGALS1, LRG1, LY86, MGAT4A, P2RY5, PRSS21, PTH2R, RNASE2, SLC43A3, SUCNR1, TIMP1, TNF, TNFRSF4, TNFSF13B, TYROBP and VNN1; cell cycle-related genes consisting of ZWINT, NEK6 and TXNL4B; an apoptosis-related gene consisting of BIK; signaling-related genes consisting of AK5, ARHGAP18, FYB, HCK, LPXN, PDE9A, PDK1, PRKCD, RAB20, RAB8A and RABIF; transcription factor genes consisting of WT1 and HLX; and other genes consisting of CYBB, CTSC and NCF4.
[3] A therapeutic agent for acute myeloid leukemia that targets leukemic stem cells, comprising as an active ingredient a substance capable of suppressing the expression of a gene selected from among leukemic stem cell marker genes consisting of the following set of genes:
cell membrane- or extracellularly-localized genes consisting of ADFP, ALOX5AP, AZU1, C3AR1, CACNB4, CALCRL, CCL4, CCL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, IL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT; apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C17orf60, C18orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, S100P, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT1, WSB2 and ZNF253;
or a substance capable of suppressing the activity of a translation product of the gene.
[4] The therapeutic agent according to [3], wherein the leukemic stem cell marker gene is selected from the group consisting of
cell membrane- or extracellularly-localized genes consisting of ADFP, ALOX5AP, CACNB4, CCL5, CD33, CD3D, CD93, CD97, CLEC12A, DOK2, FCER1G, FCGR2A, FUCA2, GPR34, GPR84, HCST, HGF, HOMER3, IL2RA, IL2RG, IL3RA, ITGB2, LGALS1, LRG1, LY86, MGAT4A, P2RY5, PRSS21, PTH2R, RNASE2, SLC43A3, SUCNR1, TIMP1, TNF, TNFRSF4, TNFSF13B, TYROBP and VNN1; cell cycle-related genes consisting of ZWINT, NEK6 and TXNL4B; an apoptosis-related gene consisting of BIK; signaling-related genes consisting of AK5, ARHGAP18, FYB, HCK, LPXN, PDE9A, PDK1, PRKCD, RAB20, RAB8A and RABIF; transcription factors consisting of WT1 and HLX; and other genes consisting of CYBB, CTSC and NCF4.
[5] The therapeutic agent according to [3], wherein the leukemic stem cell marker gene is selected from the group consisting of
cell membrane- or extracellularly-localized genes consisting of ALOX5AP, CACNB4, CCL5, CD33, CD3D, CD93, CD97, CLEC12A, DOK2, FCGR2A, GPR84, HCST, HOMER3, ITGB2, LGALS1, LRG1, PTH2R, RNASE2, TNF, TNFSF13B, TYROBP and VNN1; a cell cycle-related gene consisting of NEK6; an apoptosis-related gene consisting of BIK; signaling-related genes consisting of AK5, FYB, HCK, LPXN, PDE9A, PDK1, PRKCD and RAB20; a transcription factor gene consisting of WT1; and other genes consisting of CTSC and NCF4.
[6] The therapeutic agent according to [3], wherein the leukemic stem cell marker gene is a marker expressed in stem cells that are present in bone marrow niches, are in the stationary phase of cell cycle, and are resistant to anticancer agents, selected from the group consisting of AK5, BIK, DOK2, FCGR2A, IL2RA, LRG1, SUCNR1 and WT1.
[7] The therapeutic agent according to any one of [3] to [6], wherein the substance capable of suppressing the expression of the gene is an antisense nucleic acid or an RNAi-inducible nucleic acid.
[8] The therapeutic agent according to any one of [3] to [6], wherein the substance capable of suppressing the activity of a translation product is an aptamer or an antibody.
[9] The therapeutic agent according to [8], wherein the antibody is an immunoconjugate of an antibody and an anticancer substance.
[10] A production method of a sample containing hematopoietic cells for autologous transplantation or allogeneic transplantation for a patient with acute myeloid leukemia, comprising:

a) a step of collecting a sample containing hematopoietic cells from the patient or a donor;

b) a step of bringing the collected sample into contact with a substance that recognizes a translation product of at least one kind of leukemic stem cell marker gene selected from among the following set of genes:

cell membrane- or extracellularly-localized genes consisting of ADFP, ALOX5AP, AZU1, C3AR1, CACNB4, CALCRL, CCL4, CCL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, TL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT; apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C17orf60, C18orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, S100P, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT1, WSB2 and ZNF253; and

c) a step of sorting cells to which the substance has bound, and obtaining the sample from which leukemic stem cells have been purged.

[11] The production method according to [10], wherein the leukemic stem cell marker is at least one kind of cell surface marker gene selected from among ADFP, ALOX5AP, CACNB4, CD33, CD3D, CD93, CD97, CLEC12A, DOK2, FCER1G, FCGR2A, GPR34, GPR84, HCST, HOMER3, IL2RA, IL2RG, IL3RA, ITGB2, LY86, P2RY5, PTH2R, SUCNR1, TNFRSF4, TYROBP and VNN1.
[12] A method for preventing or treating acute myeloid leukemia that targets leukemic stem cells, comprising administering, to a subject, an effective amount of a substance capable of suppressing the expression of a gene selected from among leukemic stem cell marker genes consisting of the following set of genes:
cell membrane- or extracellularly-localized genes consisting of ADFP, ALOX5AP, AZU1, C3AR1, CACNB4, CALCRL, CCL4, COL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, IL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT;
apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C17orf60, C18orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, S100P, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT4, WSB2 and ZNF253;
or a substance capable of suppressing the activity of a translation product of the gene.

### Effect of the Invention

The present invention has been developed as a result of succeeding in analyzing the comprehensive expression profiling of leukemic stem cells (LSCs) derived from human primary AML, and identifying LSC-specific targets for separating LSCs from HSCs. Therefore, the leukemic stem cell markers found in the present invention make it possible not only to distinguish between non-stem cells and LSCs, but also to distinguish between normal hematopoietic stem cells (HSCs) and LSCs, which have been thought to be difficult to distinguish from each other. By using a leukemic stem cell marker found in the present invention as a molecular target, a therapeutic agent that acts specifically on LSCs that are the source of onset or recurrence of AML can be provided.

Also, it is possible to specifically remove LSCs from bone marrow cells of a patient or a donor using a cell sorter such as FACS, with a leukemic stem cell marker found in the present invention as an index. This will lead to the effective removal of the true source of onset or recurrences of AML. Therefore, recurrences of AML can be prevented significantly.

Furthermore, the presence or absence of LSCs in a collected biological sample or in a body can be determined with a leukemic stem cell marker found in the present invention as an index, whereby recurrences or the initial onset of acute myeloid leukemia can also be predicted.

### BRIEF DESCRIPTION OF THE DRAWINGS

FIG. 1 shows the results of transplantation of normal CD34+CD38− HSCs and AML CD34+CD38− LSCs. (Upper panel) Transplantation of normal CD34+CD38− cells resulted in efficient reconstitution of human CD45+ hematopoietic cells. Because differentiation into normal human immunocytes such as CD11c+ ordinary dendritic cells, CD123-high plasmacytoid dendritic cells, T cells and B cells is observed in human CD45+ cells, it is seen that the CD34+CD38− are hematopoietic stem cells. (Lower panel) When AML CD34+CD38− cells were transplanted, AML developed in recipient mice. Recipient BM was completely occupied by human CD45+ cells, rather than by mouse cells. Because the transplanted human cells did not contain any of normal immunocyte subsets such as dendritic cells, T cells or B cells, the CD34+CD38− cells were shown to contain no normal hematopoietic stem cells and were identified as leukemic stem cells.

FIG. 2 shows genes expressed in larger amounts in AML CD34+CD38− LSCs than in normal CD34+CD38− HSCs. The heat map includes qPCR data on 35 prominent LSC markers: 1) their functions and localization are suitable for the development of anti-AML drugs, 2) their mRNA contents are significantly (P<0.05) higher in LSCs than in HSCs, 3) the median of their mRNA contents are 5 times or more higher in LSCs than in HSCs, and 4) their mRNA contents are higher in all LSC samples tested than in various HSC samples. In this panel, red, yellow and green indicate high, moderate, and low expression, respectively, as shown by the reference color code in the lower left in this figure. Value 1 indicates the mean for mRNAs in CD34+CD38− HSCs.

FIG. 3 shows flow cytometry. The expression of LSC-specific molecule candidates (CD32, ITGB2, CD93 and CD33) was analyzed by flow cytometry. Each histogram shows relative expression in LSCs obtained from five AML patients versus that in normal HSCs.

FIG. 4 shows the results of functional assay and histological experiments of CD32. The expression of CD32 and the expression of CD133 were again analyzed by FACS. According to the expression pattern of CD32, AML patients were classified under the categories AML-a and AML-b. Normal HSCs were identified exclusively in the CD32− fraction. Likewise, leukemia induction activity was observed in the CD32− fraction of the AML-a group. In contrast, in AML-b, CD32+ cells exhibited the capability of initiating AML in vivo. In AML-b, CD32+ cells were detected in both the membrane region and central region of the bone marrow.

FIG. 5 shows heat map charts of gene candidates whose transcription products are more highly expressed in AML CD34+CD38− LSCs than in normal CD34+CD38− HSCs. 217 genes were classified on the basis of gene ontology under six categories: 1) cell membrane and extracellular, 2) cell cycle, 3) apoptosis, 4) signaling, 5) transcription factors, and 6) others. Gene expression levels on two microarray platforms (U133 plus 2.0 and Gene 1.0ST) are separately shown. In each panel, red, yellow and green indicate high, moderate and low expression, respectively.

FIG. 6 is a flow cytometric representation showing that the expression of CD32, one of the above-described candidate genes, does not undergo down regulation in AML patients after chemotherapy.

FIG. 7 shows immunofluorescent staining of the expression of various marker genes in leukemic stem cells that are present in bone marrow niches and are in the stationary phase of cell cycle. The results for each gene are shown with a set of four photographs obtained using the DAPI antibody (nuclear staining) for blue staining in the upper left, an antibody against the marker for red staining in the lower left, and an antibody against the cell cycle marker CD34 (in the case of FCGR2A, AK5, DOK2, LRG1, BIK) or the Ki67 antibody (in the case of IL2RA, WT1, SUCNR1) for green staining in the upper right. Shown in the lower right are merged results.

### MODES FOR EMBODYING THE INVENTION

### Definitions

In the present invention, the initial onset of leukemia refers to a state in which leukemia has developed for the first time, or is likely to develop, and a recurrence of leukemia refers to a state in which leukemia has developed again, or is likely to develop, after treatment or remission of initial-onset leukemia. The tissue where leukemia recurs or is likely to recur is not limited to initial-onset tissue, and may be another tissue. Therefore, the concept of recurrence is understood to include infiltration and metastasis.

In the present invention, treatment of leukemia encompasses all treatments, including administration of anticancer agents, radiotherapy, and bone marrow transplantation.

In the present invention, leukemic stem cells (LSC) may be a CD34+ cell fraction derived from the bone marrow, with preference given to CD34+CD38− cell fraction. The crude substance containing LSC can be recovered from the bone marrow of a test subject or patient by a conventional method, cell fractions containing the LSC can be obtained by flow cytometry and the like using CD34 and CD38 cell surface marker molecules. Note that separation of LSC from HSC is difficult. Furthermore, it is also possible to further sort LSCs with another cell surface marker molecule selected from among leukemic stem cell markers found by the present invention, as an index.

### (Test Method)

The present invention provides a test method for predicting the initial onset or a recurrence of acute myeloid leukemia. The test method of the present invention comprises,

(1) a step of measuring the expression level of leukemic stem cell marker genes in a biological sample collected from a subject for a transcription product or translation product of the gene as an analyte, and
(2) a step of comparing the expression levels obtained in the measuring step with the expression level in healthy persons.
(1) Step of measuring the expression level of leukemic stem cell marker genes in a biological sample collected from a subject for a transcription product or translation product of the gene as an analyte

Leukemic stem cell marker genes targeted in the present invention are leukemic stem cell-specific markers sorted from a set of genes expressed differentially in the CD34+CD38− cell fraction than in the CD34+CD38+ cell fraction by the present inventors on the basis of their unique viewpoint, and comprise 2 to 218 genes selected from among the following leukemic stem cell marker genes (hereinafter sometimes simply abbreviated as “marker genes” or “markers”) (1). The marker genes (1) preferably consist of 3 or more, 5 or more, 10 or more, 15 or more, 20 or more, or 25 or more, genes.

Marker Genes (1):

cell membrane- or extracellularly-localized genes consisting of ADFP, ALOX5AP, AZU1, C3AR1, CACNB4, CALCRL, CCL4, CCL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, IL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT; apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C17orf60, C18orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, S100P, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT1, WSB2 and ZNF253.

The individual genes that constitute the aforementioned leukemic stem cell marker genes are publicly known, and the base sequences and amino acid sequences thereof are also known.

For the marker genes except IL2RA, symbol names, gene IDs, location chromosomes, characteristics and the like are shown in Table 1. IL2RA, also called CD25 has the gene ID 3559, is located on chromosome 10, and encodes interleukin 2 receptor alpha. The IL2RA protein is a transmembranous receptor localized on the cell membrane.

TABLE 1

| | | | | Fold Change | Fold Change | | | | | |
|---|---|---|---|---|---|---|---|---|---|---|
| | | | probeID | (AML/Healthy, | (AML/Healthy, | | | | | |
| symbol | geneID | probeID U133 | GeneST | U133) | GeneST) | chromosome | explanation | function | location | process |
| ACTR2 | 10097 | 1558015_s_at | 8042337 | 4.2638 | 1.8188 | 2 | ARP2 actin-related protein 2 homolog | | cytoplasm | |
| | | | | | | | (yeast) | | | |
| ADFP | 123 | 209122_at | 8160297 | 3.2797 | 2.3209 | 9 | adipose differentiation-related protein | | cell membrane | |
| AK6 | 26289 | 222862_s_at | 7902452 | 3.1895 | 16.5796 | 1 | adenylate kinase 5 | signaling molecule | cytoplasm | |
| ALOX5 | 240 | 204446_s_at | 7927215 | 18.4004 | 2.6463 | 10 | arachidonate 5-lipoxygenase | | cytoplasm | |
| ALOX5AP | 241 | 204174_at | 7968344 | 2.8061 | 9.5599 | 13 | arachidonate 5-lipoxygenase-activating | | cell membrane | |
| | | | | | | | protein | | | |
| ANXA2P2 | 304 | 208816_x_at | 8154836 | 4.5495 | 4.8032 | 9 | annexin A2 pseudogene 2 | | unknown | |
| ARHGAP18 | 93663 | 225171_at | 8129458 | 4.3548 | 2.5895 | 6 | Rho GTPase activating protein 18 | signaling molecule | unknown | |
| ARRB1 | 408 | 222912_at | 7950473 | 3.8043 | 1.9404 | 11 | arrestin, beta 1 | signaling molecule | cytoplasm | |
| ATL3 | 25923 | 223452_s_at | 7948997 | 3.6187 | 1.8605 | 11 | atlastin 3 | | unknown | |
| ATP6V1B2 | 526 | 201089_at | 8144931 | 4.0733 | 2.0284 | 8 | ATPase, H+ transporting, lysosomal | | cytoplasm | |
| | | | | | | | 56/58 kDa, V1 subunit B2 | | | |
| ATP6V1C1 | 528 | 202872_at | 8147724 | 3.2006 | 2.3894 | 8 | ATPase, H+ transporting, lysosomal | | cytoplasm | |
| | | | | | | | 42 kDa, V1 subunit C1 | | | |
| ATP6V1D | 51382 | 208899_x_at | 7979698 | 4.6313 | 2.2873 | 14 | ATPase, H+ transporting, lysosomal | | cytoplasm | |
| | | | | | | | 34 kDa, V1 subunit D | | | |
| AURKA | 6790 | 208079_s_at | 8067167 | 2.0444 | 2.4771 | 20 | aurora kinase A | signaling molecule | nucleus | cell cycle |
| AZU1 | 566 | 214575_s_at | 8024038 | 3.3641 | 4.0737 | 19 | azurocidin 1 | | extracellular | |
| | | | | | | | | | space | |
| BIK | 638 | 205780_at | 8073605 | 5.3934 | 8.8991 | 22 | BCL2-interacting killer (apoptosis- | | cytoplasm | apoptosis |
| | | | | | | | inducing) | | | |
| C12orf5 | 57103 | 219099_at | 7953211 | 6.1678 | 4.4117 | 12 | chromosome 12 open reading frame 5 | | unknown | |
| C13orf34 | 79866 | 219544_at | 7969374 | 3.7631 | 2.3038 | 13 | chromosome 13 open reading frame 34 | | unknown | cell cycle |
| C17orf60 | 284021 | 217513_at | 8009243 | 3.2027 | 3.7201 | 17 | chromosome 17 open reading frame 60 | | unknown | |
| C18orf19 | 125228 | 235022_at | 8022404 | 2.828 | 1.8669 | 18 | chromosome 18 open reading frame 19 | | unknown | |
| C1GALT1C1 | 29071 | 219283_at | 8174820 | 4.6168 | 2.2689 | X | C1GALT1-specific chaperone 1 | | unknown | |
| C1orf135 | 79000 | 220011_at | 7913852 | 2.8168 | 2.9438 | 1 | chromosome 1 open reading frame 135 | | unknown | |
| C1orf163 | 65260 | 219420_s_at | 7916219 | 2.9798 | 2.4301 | 1 | chromosome 1 open reading frame 163 | | unknown | |
| C1orf186 | 440712 | 230381_at | 7923875 | 10.2556 | 2.3674 | 1 | chromosome 1 open reading frame 186 | | unknown | |
| C3AR1 | 719 | 209906_at | 7960874 | 8.2753 | 4.4025 | 12 | complement component 3a receptor 1 | transmembranous | cell membrane | |
| | | | | | | | | receptor | | |
| C6orf150 | 115004 | 1559051_s_at | 8127534 | 2.9862 | 3.4927 | 6 | chromosome 6 open reading frame 150 | | unknown | |
| CACNB4 | 785 | 207693_at | 8055872 | 2.8303 | 2.6543 | 2 | calcium channel, voltage-dependent, beta | | cell membrane | |
| | | | | | | | 4 subunit | | | |
| CALCRL | 10203 | 206331_at | 8057578 | 2.5601 | 2.2268 | 2 | calcitonin receptor-like | transmembranous | cell membrane | |
| | | | | | | | | receptor | | |
| CALML4 | 91860 | 221879_at | 7989968 | 2.5411 | 2.2694 | 15 | calmodulin-like 4 | | unknown | |
| CCL4 | 6351 | 204103_at | 8006602 | 12.0201 | 2.1087 | 17 | chemokine (C-C motif) ligand 4 | cytokine and | extracellular | |
| | | | | | | | | growth factor | space | |
| CCL5 | 6352 | 1405_i_at | 8014316 | 13.0433 | 10.0074 | 17 | chemokine (C-C motif) ligand 5 | cytokine and | extracellular | immunity, cell adhesion |
| | | | | | | | | growth factor | space | |
| CCNA1 | 8900 | 205899_at | 7968637 | 3.325 | 3.9705 | 13 | cyclin A1 | | nucleus | cell cycle |
| CCT5 | 22948 | 229068_at | 8104449 | 2.828 | 2.0069 | 5 | chaperonin containing TCP1, subunit 5 | | cytoplasm | |
| | | | | | | | (epsilon) | | | |
| CD33 | 945 | 206120_at | 8030804 | 3.4258 | 4.0167 | 19 | CD33 molecule | signaling molecule | cell membrane | cell adhesion |
| CD36 | 948 | 228766_at | 8133876 | 6.3287 | 2.1815 | 7 | CD36 molecule (thrombospondin | | cell membrane | |
| | | | | | | | receptor) | | | |
| CD3D | 915 | 213539_at | 7952056 | 6.673 | 11.2019 | 11 | CD3d molecule, delta (CD3-TCR | transmembranous | cell membrane | |
| | | | | | | | complex) | receptor | | |
| CD86 | 942 | 210895_s_at | 8082035 | 3.6193 | 4.1863 | 3 | CD86 molecule | transmembranous | cell membrane | |
| | | | | | | | | receptor | | |
| CD9 | 928 | 201005_at | 7953291 | 28.019 | 1.7512 | 12 | CD9 molecule | | cell membrane | |
| CD93 | 22918 | 202878_s_at | 8065359 | 13.7302 | 1.9706 | 20 | CD93 molecule | | cell membrane | cell adhesion |
| CD96 | 10225 | 206761_at | 8081564 | 4.247 | 4.9945 | 3 | CD96 molecule | | cell membrane | |
| CD97 | 976 | 202910_s_at | 8026300 | 7.6085 | 2.0902 | 19 | CD97 molecule | transmembranous | cell membrane | immunity, cell adhesion |
| | | | | | | | | receptor | | |
| CFD | 1675 | 205382_s_at | 8024062 | 7.7147 | 3.955 | 19 | complement factor D (adipsin) | | extracellular | |
| | | | | | | | | | space | |
| CHI3L1 | 1116 | 209395_at | 7923547 | 3.3299 | 2.3625 | 1 | chitinase 3-like 1 (cartilage glycoprotein- | | extracellular | |
| | | | | | | | 39) | | space | |
| CLC | 1178 | 206207_at | 8036755 | 4.9719 | 20.5111 | 19 | Charcot-Leyden crystal protein | | cytoplasm | |
| CLEC12A | 160364 | 1552398_a_at | 7953901 | 14.7668 | 10.4421 | 12 | C-type lectin domain family 12, member A | | cell membrane | |
| CLECL1 | 160365 | 244413_at | 7961069 | 3.2716 | 9.2279 | 12 | C-type lectin-like 1 | | cell membrane | |
| COCH | 1690 | 205229_s_at | 7973797 | 2.6217 | 2.5193 | 14 | coagulation factor C homolog, cochlin | | extracellular | |
| | | | | | | | (Limulus polyphemus) | | space | |
| COMMD8 | 54951 | 218351_at | 8100145 | 4.696 | 2.2621 | 4 | COMM domain containing 8 | | unknown | |
| COTL1 | 23406 | 224583_at | 8003171 | 4.2253 | 1.9761 | 16 | coactosin-like 1 (Dictyostelium) | | cytoplasm | |
| COX17 | 10063 | 203880_at | 7968972 | 2.9867 | 2.0975 | 3 | COX17 cytochrome c oxidase assembly | | cytoplasm | |
| | | | | | | | homolog (S. cerevisiae) | | | |
| CRIP1 | 1396 | 205081_at | 7977409 | 10.2268 | 1.9145 | 14 | cysteine-rich protein 1 (intestinal) | | cytoplasm | |
| CST7 | 8530 | 210140_at | 8061416 | 4.5721 | 4.3944 | 20 | cystatin F (leukocystatin) | | extracellular | |
| | | | | | | | | | space | |
| CSTA | 1475 | 204971_at | 8082058 | 15.2724 | 8.2554 | 3 | cystatin A (stefin A) | | cytoplasm | |
| CTSA | 5476 | 200661_at | 8063078 | 7.4704 | 2.0204 | 20 | cathepsin A | | cytoplasm | |
| CTSC | 1075 | 201487_at | 7950906 | 4.4802 | 3.1109 | 11 | cathepsin C | | cytoplasm | immunity |
| CTSG | 1511 | 205653_at | 7978351 | 4.5766 | 5.8038 | 14 | cathepsin G | | cytoplasm | immunity |
| CXCL1 | 2919 | 204470_at | 8095697 | 11.0827 | 2.1273 | 4 | chemokine (C—X—C motif) ligand 1 | cytokine and | extracellular | |
| | | | | | | | (melanoma growth stimulating | growth factor | space | |
| CYBB | 1536 | 203923_s_at | 8166730 | 3.9235 | 4.1921 | X | cytochrome b-245, beta polypeptide | | cytoplasm | immunity |
| CYP2E1 | 1571 | 209975_at | 7931643 | 2.58 | 2.2439 | 10 | cytochrome P450, family 2, subfamily E, | | cytoplasm | |
| | | | | | | | polypeptide 1 | | | |
| DENND3 | 22898 | 212975_at | 8148476 | 2.9132 | 2.0985 | 8 | DENN/MADD domain containing 3 | | unknown | |
| DHRS3 | 9249 | 202481_at | 7912537 | 3.7799 | 2.499 | 1 | dehydrogenase/reductase (SDR family) | | cytoplasm | |
| | | | | | | | member 3 | | | |
| DLAT | 1737 | 212568_s_at | 7943827 | 5.313 | 2.2002 | 11 | dihydrolipoamide S-acetyltransferase | | cytoplasm | |
| DLEU2 | 8847 | 1556821_x_at | 7971653 | 2.876 | 3.9304 | 13 | deleted in lymphocytic leukemia 2 (non- | | unknown | |
| | | | | | | | protein coding) | | | |
| DOK2 | 9046 | 214054_at | 8149638 | 5.6934 | 3.0391 | 8 | docking protein 2, 56 kDa | signaling molecule | cell membrane | |
| DPH3 | 285381 | 225200_at | 8085660 | 2.875 | 2.0279 | 3 | DPH3, KTI11 homolog S. cerevisiae) | | cytoplasm | |
| DSCC1 | 79075 | 219000_s_at | 8152582 | 2.5694 | 2.6348 | 8 | defective in sister chromatid cohesion 1 | | nucleus | cell cycle |
| | | | | | | | homolog (S. cerevisiae) | | | |
| DUSP6 | 1848 | 208893_s_at | 7965335 | 3.9521 | 2.0696 | 12 | dual specificity phosphatase 6 | signaling molecule | cytoplasm | |
| EFHD2 | 79180 | 222483_at | 7898161 | 3.0525 | 2.0378 | 1 | EF-hand domain family, member D2 | | unknown | |
| EMR2 | 30817 | 207610_s_at | 8034873 | 10.5352 | 2.0458 | 19 | egf-like module containing, mucin-like, | | cell membrane | |
| | | | | | | | hormone receptor-like 2 | | | |
| ENC1 | 8507 | 201341_at | 8112615 | 6.3235 | 1.8298 | 5 | ectodermal-neural cortex (with BTB-like | | nucleus | |
| | | | | | | | domain) | | | |
| EXOSO3 | 51010 | 227916_x_at | 8161242 | 3.051 | 2.019 | 9 | exosome component 3 | | nucleus | |
| FAM107B | 83641 | 223058_at | 7932160 | 11.3343 | 2.1322 | 10 | family with sequence similarity 107, | | nucleus | |
| | | | | | | | member B | | | |
| FAM129A | 116496 | 217966_s_at | 7922846 | 7.1413 | 1.714 | 1 | family with sequence similarity 129, | | cytoplasm | |
| | | | | | | | member A | | | |
| FAM33A | 348235 | 225684_at | 8017133 | 2.6048 | 2.0228 | 17 | family with sequence similarity 33, | | nucleus | cell cycle |
| | | | | | | | member A | | | |
| FAM38B | 63895 | 219602_s_at | 8022283 | 2.7684 | 2.4385 | 18 | family with sequence similarity 38, | | unknown | |
| | | | | | | | member B | | | |
| FBXO22 | 26263 | 225734_at | 7985053 | 3.3569 | 1.8734 | 15 | F-box protein 22 | | unknown | |
| FCER1G | 2207 | 204232_at | 7906720 | 5.172 | 4.5138 | 1 | Fo fragment of IgE, high affinity 1, | transmembranous | cell membrane | immunity, apoptosis |
| | | | | | | | receptor for, gamma polypeptide | receptor | | |
| FCGR2A | 2212 | 203561_at | 7906757 | 3.6163 | 4.5895 | 1 | Fc fragment of IgG, low affinity IIa, | transmembranous | cell membrane | |
| | | | | | | | receptor (CD32) | receptor | | |
| FLJ14213 | 79899 | 233379_at | 7939383 | 3.2662 | 1.8592 | 11 | protor-2 | | unknown | |
| FNDC3B | 64778 | 222692_s_at | 8083901 | 4.0438 | 1.815 | 3 | fibronectin type III domain containing 3B | | unknown | |
| FUCA2 | 2519 | 223120_at | 8129974 | 2.7625 | 2.214 | 6 | fucosidase, alpha-L-2, plasma | | extracellular | |
| | | | | | | | | | space | |
| FYB | 2533 | 227266_s_at | 8111739 | 5.75 | 3.4782 | 5 | FYN binding protein (FYB-120/130) | signaling molecule | nucleus | immunity |
| GADD45B | 4616 | 209305_s_at | 8024485 | 8.2835 | 1.8588 | 19 | growth arrest and DNA-damage- | | cytoplasm | apoptosis |
| | | | | | | | inducible, beta | | | |
| GNPDA1 | 10007 | 202382_s_at | 8114787 | 4.3678 | 1.8908 | 5 | glucosamine-6-phosphate deaminase 1 | | cytoplasm | |
| GPR109B | 8843 | 205220_at | 7967322 | 25.4362 | 5.9615 | 12 | G protein-coupled receptor 109B | transmembranous | cell membrane | |
| | | | | | | | | receptor | | |
| GPR180 | 26996 | 223423_at | 8083839 | 2.4534 | 2.4379 | 3 | G protein-coupled receptor 160 | transmembranous | cell membrane | |
| | | | | | | | | receptor | | |
| GPR34 | 2857 | 223620_at | 8166906 | 3.7631 | 2.7359 | X | G protein-coupled receptor 34 | transmembranous | cell membrane | |
| | | | | | | | | receptor | | |
| GPR84 | 53831 | 223767_at | 7963770 | 3.5766 | 2.6827 | 12 | G protein-coupled receptor 84 | transmembranous | cell membrane | |
| | | | | | | | | receptor | | |
| GRPEL1 | 80273 | 212432_at | 8099246 | 8.8722 | 2.1952 | 4 | GrpE-like 1, mitochondrial (E. coli) | | cytoplasm | |
| GTSF1 | 121355 | 227711_at | 7963817 | 8.6795 | 5.142 | 12 | gametocyte specific factor 1 | | cytoplasm | |
| HAVOR2 | 84868 | 235458_at | 8115464 | 3.8093 | 2.0482 | 5 | hepatitis A virus cellular receptor 2 | transmembranous | cell membrane | |
| | | | | | | | | receptor | | |
| HBEGF | 1839 | 203821_at | 8114572 | 19.1502 | 3.4502 | 5 | heparin-binding EGF-like growth factor | cytokine and | extracellular | |
| | | | | | | | | growth factor | space | |
| HCK | 3055 | 28018_s_at | 8061668 | 17.6625 | 4.7152 | 20 | hemopoletic cell kinase | signaling molecule | cytoplasm | |
| HCST | 10870 | 223640_at | 8028104 | 4.0478 | 2.9073 | 19 | hematopoietic cell signal transducer | | cell membrane | |
| HGF | 3082 | 210997_at | 8140556 | 4.5623 | 2.7163 | 7 | hepatocyte growth factor (hepapoietin A; | cytokine and | extracellular | |
| | | | | | | | scatter factor) | growth factor | space | |
| HIG2 | 29923 | 218507_at | 8135915 | 2.6299 | 2.0696 | 7 | hypoxia-inducible protein 2 | | unknown | |
| HLA-DOB | 3112 | 205671_s_at | 8178833 | 2.9282 | 1.8281 | 6 | major histocompatibility complex, class II, | transmembranous | cell membrane | |
| | | | | | | | DO beta | receptor | | |
| HLX | 3142 | 214438_at | 7909890 | 4.7545 | 1.972 | 1 | H2.0-like homeobox | transcription | nucleus | |
| | | | | | | | | factor | | |
| HN1 | 51155 | 217755_at | 8018305 | 3.5232 | 2.6057 | 17 | hematological and neurological expressed 1 | | nucleus | |
| HOMER3 | 9454 | 204647_at | 8035566 | 13.8417 | 4.0343 | 19 | homer homolog 3 (Drosophila) | signaling molecule | cell membrane | |
| HPGD | 3248 | 203914_x_at | 8103769 | 1.9977 | 5.1611 | 4 | hydroxyprostaglandin dehydrogenase 15- | | cytoplasm | cell cycle |
| | | | | | | | (NAD) | | | |
| HVCN1 | 84329 | 226879_at | 7966356 | 3.112 | 2.231 | 12 | hydrogen voltage-gated channel 1 | | unknown | |
| IDH1 | 3417 | 201193_at | 8058552 | 2.5681 | 2.1009 | 2 | isocitrate dehydrogenase 1 (NADP+), | | cytoplasm | |
| | | | | | | | soluble | | | |
| IDH3A | 3419 | 202069_s_at | 7985134 | 3.9339 | 2.6845 | 15 | isocitrate dehydrogenase 3 (NAD+) alpha | | cytoplasm | |
| IER3 | 8870 | 201631_s_at | 8179704 | 2.9818 | 2.6543 | 6 | immediate early response 3 | | cytoplasm | apoptosis |
| IFI30 | 10437 | 201422_at | 8026971 | 11.7514 | 3.0596 | 19 | interferon, gamma-inducible protein 30 | | extracellular | |
| | | | | | | | | | space | |
| IKIP | 121457 | 227295_at | 7965681 | 3.5724 | 1.8803 | 12 | IKK interacting protein | | unknown | |
| IL13RA1 | 3597 | 201887_at | 8169580 | 7.1957 | 2.4697 | X | interleukin 13 receptor, alpha 1 | transmembranous | cell membrane | |
| | | | | | | | | receptor | | |
| IL2RG | 3561 | 204116_at | 8173444 | 2.2169 | 2.6537 | X | interleukin 2 receptor, gamma (severe | transmembranous | cell membrane | immunity |
| | | | | | | | combined immunodeficiency) | receptor | | |
| IL3RA | 3563 | 206148_at | 8176323 | 3.392 | 2.9718 | X\|Y | interleukin 3 receptor, alpha (low affinity) | transmembranous | cell membrane | |
| | | | | | | | | receptor | | |
| INHBA | 3624 | 210511_s_at | 8139207 | 7.886 | 1.977 | 7 | inhibin, beta A | cytokine and | extracellular | |
| | | | | | | | | growth factor | space | |
| ITGB2 | 3689 | 1555349_a_at | 8070826 | 3.4371 | 2.3718 | 21 | integrin, beta 2 (complement component | signaling molecule | cell membrane | cell adhesion, apoptosis |
| | | | | | | | 3 receptor 3 and 4 | | | |
| KIF2C | 11004 | 209408_at | 7901010 | 2.4566 | 2.3144 | 1 | kinesin family member 2C | | nucleus | |
| KYNU | 8942 | 217388_s_at | 8045539 | 21.2871 | 4.5148 | 2 | kynureninase (L-kynurenine hydrolase) | | cytoplasm | |
| LCMT2 | 9836 | 204012_s_at | 7988077 | 2.8266 | 1.8921 | 15 | leucine carboxyl methyltransferase 2 | | unknown | |
| LGALS1 | 3956 | 201105_at | 8072876 | 17.9891 | 7.0421 | 22 | lectin, galactoside-binding, soluble, 1 | | extracellular | apoptosis |
| | | | | | | | | | space | |
| LPXN | 9404 | 216250_s_at | 7948332 | 6.1566 | 5.0537 | 11 | leupaxin | signaling molecule | cytoplasm | cell adhesion |
| LRG1 | 116844 | 228648_at | 8032834 | 5.7066 | 2.2013 | 19 | leucine-rich alpha-2-glycoprotein 1 | | extracellular | |
| | | | | | | | | | space | |
| LY86 | 9450 | 205859_at | 8116734 | 9.8638 | 11.3294 | 6 | lymphocyte antigen 86 | | cell membrane | immunity, apoptosis |
| MAMDC2 | 256691 | 228885_at | 8155754 | 50.0231 | 1.8485 | 9 | MAM domain containing 2 | | extracellular | |
| | | | | | | | | | space | |
| ME1 | 4199 | 204059_s_at | 8127854 | 3.167 | 6.0952 | 6 | mallo enzyme 1, NADP(+)-dependent, | | cytoplasm | |
| | | | | | | | cytosolic | | | |
| MGAT4A | 11320 | 226039_at | 8054135 | 2.488 | 2.145 | 2 | mannosyl ((alpha-1,3-)-glycoprotein beta- | | extracellular | |
| | | | | | | | 1,4-N- | | space | |
| MIRN21 | 406981 | 224917_at | 8008885 | 7.1437 | 2.2647 | 17 | microRNA 21 | | unknown | |
| MKKS | 8195 | 218138_at | 8064967 | 5.0082 | 2.1926 | 20 | McKusick-Kaufman syndrome | | cytoplasm | |
| MNDA | 4332 | 204959_at | 7906377 | 7.9908 | 6.7427 | 1 | myeloid cell nuclear differentiation | | nucleus | |
| | | | | | | | antigen | | | |
| MPO | 4353 | 203949_at | 8016932 | 3.4405 | 2.9167 | 17 | myeloperoxidase | | cytoplasm | apoptosis |
| MS4A3 | 932 | 210254_at | 7940216 | 2.9166 | 6.6468 | 11 | membrane-spanning 4-domains, | signaling molecule | cytoplasm | |
| | | | | | | | subfamily A, member 3 | | | |
| MTHFD2 | 10797 | 201761_at | 8042830 | 2.7123 | 1.9426 | 2 | methylenetetrahydrofolate | | cytoplasm | |
| | | | | | | | dehydrogenase (NADP+ dependent) 2, | | | |
| MYC | 4609 | 202431_s_at | 8148317 | 4.8528 | 1.9292 | 8 | v-myo myelocytomatosis viral oncogene | transcription | nucleus | |
| | | | | | | | homolog (avian) | factor | | |
| MYO1B | 4430 | 212364_at | 8047127 | 3.1023 | 1.9101 | 2 | myosin 1B | | cytoplasm | |
| MYO1F | 4542 | 213733_at | 8033605 | 3.93 | 2.5391 | 19 | myosin 1F | | cytoplasm | |
| NAGA | 4668 | 202943_s_at | 8076403 | 2.7565 | 2.4116 | 22 | N-acetylgalactosaminidssa, alpha- | | cytoplasm | |
| NAIP | 4671 | 239944_at | 8177527 | 3.9106 | 2.0172 | 5 | NLR family, apoptosis inhibitory protein | | cytoplasm | apoptosis |
| NCF2 | 4688 | 209949_at | 7922773 | 8.0756 | 3.4526 | 1 | neutrophil cytosolic factor 2 | | cytoplasm | |
| NCF4 | 4689 | 205147_x_at | 8072744 | 3.0753 | 3.3081 | 22 | neutrophil cytosolic factor 4, 40 kDa | | cytoplasm | immunity |
| NDUFAF1 | 51103 | 204125_at | 7987642 | 4.6031 | 2.1624 | 15 | NADH dehydrogenase (ubiquinone) 1 | | cytoplasm | |
| | | | | | | | alpha subcomplex, assembly factor | | | |
| NEK6 | 10783 | 223159_s_at | 8157761 | 5.1566 | 3.4558 | 9 | NIMA (never in mitosis gene a)-related | signaling molecule | nucleus | cell cycle, apoptosis |
| | | | | | | | kinase 6 | | | |
| NP | 4860 | 201695_s_at | 7973067 | 8.9531 | 2.1755 | 14 | nucleoside phosphorylase | | nucleus | |
| NRIP3 | 56675 | 219557_s_at | 7946446 | 3.2732 | 3.6133 | 11 | nuclear receptor interacting protein 3 | | unknown | |
| OBFC2A | 64859 | 222872_x_at | 8047161 | 13.5591 | 2.7032 | 2 | oligonucleotide/oligosaccharide-binding | | nucleus | |
| | | | | | | | fold containing 2A | | | |
| P2RY14 | 9934 | 206637_at | 8091511 | 3.0798 | 2.4295 | 3 | purinergic receptor P2Y, G-protein | transmembranous | cell membrane | |
| | | | | | | | coupled, 14 | receptor | | |
| P2RY5 | 10161 | 218589_at | 7971585 | 1.5532 | 2.7233 | 13 | purinergic receptor P2Y, G-protein | transmembranous | cell membrane | |
| | | | | | | | coupled, 5 | receptor | | |
| PAK1IP1 | 55003 | 218886_at | 8116848 | 4.0989 | 2.4834 | 6 | PAK1 interacting protein 1 | signaling molecule | nucleus | |
| PARP8 | 79668 | 219033_at | 8105191 | 7.2902 | 2.9745 | 5 | poly (ADP-ribose) polymerase family, | | nucleus | |
| | | | | | | | member 8 | | | |
| PDE9A | 5152 | 205593_s_at | 8068833 | 5.8044 | 3.5595 | 21 | phosphodiesterase 9A | signaling molecule | cytoplasm | |
| PDK1 | 5163 | 226452_at | 8046408 | 2.6927 | 2.4033 | 2 | pyruvate dehydrogenase kinase, isozyme 1 | signaling molecule | cytoplasm | |
| PDLIM1 | 9124 | 208690_s_at | 7935180 | 15.0721 | 1.5551 | 10 | PDZ and LIM domain 1 | | cytoplasm | |
| PDSS1 | 23590 | 220865_s_at | 7926807 | 2.7366 | 2.4594 | 10 | prenyl (decaprenyl) diphosphate | | unknown | |
| | | | | | | | synthase, subunit 1 | | | |
| PGM2 | 55276 | 225366_at | 8094556 | 3.8421 | 2.1135 | 4 | phosphoglucomutase 2 | | cytoplasm | |
| PIGK | 10026 | 209707_at | 7917088 | 4.7506 | 2.9006 | 1 | phosphatidylinositol glycan anchor | | cytoplasm | |
| | | | | | | | biosynthesis, class K | | | |
| PIWIL4 | 143689 | 230480_at | 7943240 | 3.308 | 1.9009 | 11 | piwi-like 4 (Drosophila) | | unknown | |
| PLAUR | 5329 | 210845_s_at | 8037374 | 6.5367 | 1.697 | 19 | plasminogen activator, urokinase receptor | transmembranous | cell membrane | |
| | | | | | | | | receptor | | |
| PPBP | 5473 | 214146_s_at | 8100971 | 12.1498 | 1.5966 | 4 | pro-platelet basic protein (chemokine | cytokine and | extracellular | |
| | | | | | | | (C—X—C motif) ligand 7) | growth factor | space | |
| PPCDC | 60490 | 219066_at | 7984943 | 3.3971 | 2.0648 | 15 | phosphopantothenoylcysteine | | unknown | |
| | | | | | | | decarboxylase | | | |
| PPIF | 10105 | 201489_at | 7928589 | 5.105 | 2.4099 | 10 | peptidylprolyl isomerase F (cyclophilin F) | | cytoplasm | |
| PRAME | 23532 | 204086_at | 8074856 | 2.9345 | 7.1984 | 22 | preferentially expressed antigen in | | nucleus | |
| | | | | | | | melanoma | | | |
| PRG2 | 5553 | 211743_s_at | 7948221 | 5.7443 | 5.1313 | 11 | proteoglycan 2, bone marrow (natural | | extracellular | |
| | | | | | | | killer cell activator. | | space | |
| PRKAR1A | 5573 | 200604_s_at | 8009457 | 2.5728 | 1.9221 | 17 | protein kinase, cAMP-dependent, | signaling molecule | cytoplasm | |
| | | | | | | | regulatory, type 1, alpha (tissue | | | |
| PRKCD | 5580 | 202545_at | 8080487 | 11.8684 | 4.4878 | 3 | protein kinase C, delta | signaling molecule | cytoplasm | |
| PRSS21 | 10942 | 220051_at | 7992722 | 2.8945 | 2.4783 | 16 | protease, serine, 21 (testisin) | | extracellular | |
| | | | | | | | | | space | |
| PTH2R | 5746 | 206772_at | 8047910 | 32.9485 | 2.6488 | 2 | parathyroid hormone 2 receptor | transmembranous | cell membrane | |
| | | | | | | | | receptor | | |
| PTX3 | 5806 | 206157_at | 8083594 | 1.371 | 1.7203 | 3 | pentraxin-related gene, rapidly induced | | extracellular | |
| | | | | | | | by IL-1 beta | | space | |
| PUS7 | 54517 | 218984_at | 8142061 | 3.473 | 2.0097 | 7 | pseudouridylate synthase 7 homolog | | unknown | |
| | | | | | | | (S. cerevisiae) | | | |
| PXK | 54899 | 1552275_s_at | 8080781 | 2.9549 | 2.2995 | 3 | PX domain containing serine/threonine | signaling molecule | cytoplasm | |
| | | | | | | | kinase | | | |
| PYHIN1 | 149628 | 240413_at | 7906386 | 3.6814 | 1.9866 | 1 | pyrin and HIN domain family, member 1 | | nucleus | cell cycle |
| RAB20 | 55647 | 219622_at | 7972805 | 4.2758 | 1.9982 | 13 | RAB20, member RAS oncogene family | signaling molecule | cytoplasm | |
| RAB8A | 4218 | 208819_at | 8026520 | 3.089 | 2.0472 | 19 | RAB8A, member RAS oncogene family | signaling molecule | cytoplasm | |
| RABIF | 5877 | 204478_s_at | 7923483 | 6.673 | 1.9163 | 1 | RAB interacting factor | signaling molecule | unknown | |
| RASGRP3 | 25780 | 205801_s_at | 8041422 | 12.2403 | 2.3489 | 2 | RAS guanyl releasing protein 3 (calcium | signaling molecule | cytoplasm | |
| | | | | | | | and DAG-regulated) | | | |
| RASSF4 | 83937 | 226436_at | 7927186 | 5.1219 | 1.8566 | 10 | Ras association (RalGDS/AF-6) domain | | unknown | cell cycle |
| | | | | | | | family member 4 | | | |
| PEEP5 | 7905 | 208873_s_at | 8113542 | 1.9895 | 2.4938 | 5 | receptor accessory protein 5 | | extracellular | |
| | | | | | | | | | space | |
| RGS18 | 64407 | 223809_at | 7908376 | 18.2071 | 3.0532 | 1 | regulator of G-protein signaling 18 | signaling molecule | cytoplasm | |
| RNASE2 | 6036 | 206111_at | 7973110 | 6.2056 | 30.3509 | 14 | ribonuclease, RNase A family, 2 (liver, | | extracellular | |
| | | | | | | | eosinophil-derived | | space | |
| RPP40 | 10799 | 213427_at | 8123717 | 2.5261 | 2.3867 | 6 | ribonuclease P/MRP 40 kDa subunit | | nucleus | |
| RRM2 | 6241 | 201890_at | 8040223 | 1.8022 | 1.9629 | 2 | ribonucleotide reductase M2 polypeptide | | cytoplasm | |
| RXFP1 | 59350 | 231804_at | 8098060 | 8.4366 | 4.7218 | 4 | relaxin/insulin-like family peptide | transmembranous | cell membrane | |
| | | | | | | | receptor 1 | receptor | | |
| S100A11 | 6282 | 200660_at | 7920128 | 2.6989 | 1.9757 | 1 | S100 calcium binding protein A11 | signaling molecule | cytoplasm | |
| S100A16 | 140576 | 227998_at | 7920291 | 6.5974 | 5.2295 | 1 | S100 calcium binding protein A16 | | nucleus | |
| S100A8 | 6279 | 202917_s_at | 7920244 | 3.7254 | 4.1401 | 1 | S100 calcium binding protein A8 | | cytoplasm | |
| S100P | 6286 | 204351_at | 8093950 | 2.8439 | 4.35 | 4 | S100 calcium binding protein P | | cytoplasm | |
| S100Z | 170591 | 1554876_s_at | 8106411 | 2.5656 | 3.2588 | 5 | S100 calcium binding protein Z | | unknown | |
| SAMHD1 | 25939 | 204502_at | 8066117 | 3.5478 | 3.315 | 20 | SAM domain and HD domain 1 | | nucleus | immunity |
| SH2D1A | 4068 | 210116_at | 8169792 | 5.4768 | 4.944 | X | SH2 domain protein 1A | | cytoplasm | |
| SLC31A2 | 1318 | 204204_at | 8157264 | 6.5551 | 1.9871 | 9 | solute carrier family 31 (copper | | cell membrane | |
| | | | | | | | transporters), member 2 | | | |
| SLC43A3 | 29016 | 213113_s_at | 7948229 | 4.0265 | 2.5036 | 11 | solute carrier family 43, member 3 | | extracellular | |
| | | | | | | | | | space | |
| SLC6A6 | 6533 | 211030_s_at | 8078014 | 3.3096 | 1.9332 | 3 | solute carrier family 6 (neurotransmitter | | cell membrane | |
| | | | | | | | transporter, | | | |
| SLC7A6 | 9057 | 203579_s_at | 7996772 | 2.537 | 2.0624 | 16 | solute carrier family 7 (cationic amino | | cell membrane | |
| | | | | | | | acid transporter, y+ | | | |
| SPCS2 | 9789 | 201239_s_at | 7914180 | 2.8247 | 2.8859 | 11 | signal peptidase complex subunit 2 | | cytoplasm | |
| | | | | | | | homolog (S. cerevisiae) | | | |
| SPPL2A | 84888 | 226353_at | 7988753 | 3.6826 | 3.1645 | 15 | signal peptide peptidase-like 2A | | unknown | |
| STX7 | 8417 | 212631_at | 8129590 | 3.044 | 1.957 | 6 | syntaxin 7 | | cell membrane | |
| SUCNR1 | 56670 | 223939_at | 8083422 | 10.3593 | 8.9094 | 3 | succinate receptor 1 | transmembranous | cell membrane | |
| | | | | | | | | receptor | | |
| TACSTD2 | 4070 | 202286_s_at | 7916584 | 4.5271 | 2.3856 | 1 | tumor-associated calcium signal | | cell membrane | |
| | | | | | | | transducer 2 | | | |
| TESC | 54997 | 218872_at | 7966749 | 6.7818 | 4.194 | 12 | tescalcin | | unknown | |
| THEX1 | 90459 | 226416_at | 8144516 | 3.539 | 2.084 | 8 | three prime histone mRNA exonuclease 1 | | unknown | |
| TIMP1 | 7076 | 201666_at | 8167185 | 2.6582 | 1.8176 | X | TIMP metallopeptidase inhibitor 1 | | extracellular | |
| | | | | | | | | | space | |
| TM4SF1 | 4071 | 215034_s_at | 8091411 | 13.9334 | 3.1159 | 3 | transmembrane 4 L six family member 1 | | cell membrane | |
| TM9SF1 | 10548 | 209149_s_at | 7978166 | 3.8307 | 1.9283 | 14 | transmembrane 9 superfamily member 1 | | cell membrane | |
| TMEM30A | 55754 | 232591_s_at | 8127637 | 3.436 | 1.8579 | 6 | transmembrane protein 30A | | unknown | |
| TMEM33 | 55161 | 218465_at | 8094830 | 2.7565 | 2.4022 | 4 | transmembrane protein 33 | | unknown | |
| TNF | 7124 | 207113_s_at | 8179263 | 5.16 | 3.3831 | 6 | tumor necrosis factor (TNF superfamily, | cytokine and | extracellular | immunity, apoptosis |
| | | | | | | | member 2) | growth factor | space | |
| TNFRSF4 | 7293 | 214228_x_at | 7911413 | 4.2204 | 2.4055 | 1 | tumor necrosis factor receptor | transmembranous | cell membrane | immunity |
| | | | | | | | superfamily, member 4 | receptor | | |
| TNFSF13B | 10673 | 223501_at | 7969986 | 9.7537 | 2.3209 | 13 | tumor necrosis factor (ligand) | cytokine and | extracellular | immunity |
| | | | | | | | superfamily, member 13b | growth factor | space | |
| TRIP13 | 9319 | 204033_at | 8104234 | 2.146 | 2.0662 | 5 | thyroid hormone receptor interactor 13 | | cytoplasm | |
| TUBB6 | 84617 | 209191_at | 8020220 | 5.4543 | 1.8033 | 18 | tubulin, beta 6 | | cytoplasm | |
| TXNDC1 | 81542 | 208097_s_at | 7974303 | 2.8632 | 1.9163 | 14 | thioredoxin domain containing 1 | | cytoplasm | apoptosis |
| TXNL4B | 54957 | 222748_s_at | 8002660 | 4.1397 | 1.9453 | 16 | thioredoxin-like 4B | | nucleus | cell cycle |
| TYROBP | 7305 | 204122_at | 8036224 | 21.8206 | 5.4076 | 19 | TYRO protein tyrosine kinase binding | signaling molecule | cell membrane | |
| | | | | | | | protein | | | |
| UBASH3B | 84959 | 238587_at | 7944722 | 3.5884 | 2.858 | 11 | ubiquitin associated and SH3 domain | | unknown | |
| | | | | | | | containing, B | | | |
| UGCG | 7357 | 221765_at | 8157216 | 4.0106 | 2.3729 | 9 | glucosyltransferase | | cytoplasm | |
| UTS2 | 10911 | 220784_s_at | 7912136 | 5.3996 | 6.4651 | 1 | urotensin 2 | | extracellular | |
| | | | | | | | | | space | |
| VNN1 | 8876 | 205844_at | 8129618 | 11.9292 | 5.0877 | 6 | vanin 1 | | cell membrane | immunity, apoptosis, |
| | | | | | | | | | | cell adhesion |
| VSTM1 | 284415 | 235818_at | 8039109 | 2.8594 | 5.2732 | 19 | V-set and transmembrane domain | | unknown | |
| | | | | | | | containing 1 | | | |
| WDR4 | 10785 | 241937_s_at | 8070615 | 2.8509 | 2.2356 | 21 | WD repeat domain 4 | | nucleus | |
| WIT1 | 51352 | 206954_at | 7939131 | 2.8415 | 2.6555 | 11 | Wilms tumor upstream neighbor 1 | | unknown | |
| WSB2 | 55884 | 201760_s_at | 7966829 | 3.5848 | 1.9757 | 12 | WD repeat and SOCS box-containing 2 | | unknown | |
| WT1 | 7490 | 206067_s_at | 7947363 | 93.6087 | 1.7707 | 11 | Wilms tumor 1 | transcription | nucleus | cell cycle |
| | | | | | | | | factor | | |
| ZNF253 | 56242 | 242919_at | 8027241 | 2.6622 | 2.176 | 19 | zinc finger protein 253 | | nucleus | |
| ZWINT | 11130 | 204026_s_at | 7933707 | 1.9796 | 2.3419 | 10 | ZW10 interactor | | nucleus | cell cycle |

When the test method of the present invention is intended to more clearly distinguish between LSCs and HSCs, it is preferable that the following marker genes (2), for example, out of the above-described marker genes (1), be used as an index. In this mode of embodiment, the marker genes (2) consist of 2 to 58 genes, more preferably consist of 3 or more, or more, 10 or more, 15 or more, 20 or more, or 25 or more, genes. When the test method of the present invention is intended to still more clearly distinguish between LSCs and HSCs, it is preferable that the following marker genes (3), out of the marker genes (2), be used as an index. The marker genes (3) are more preferable because normally 5 times or higher differential expression is observed in LSCs than in HSCs. In this mode of embodiment, the marker genes (3) consist of 2 to 35 genes, more preferably consist of 3 or more, 5 or more, 10 or more, 15 or more, 20 or more, or 25 or more, genes.

Marker Genes (2):

cell membrane- or extracellularly-localized genes consisting of ADFP, ALOX5AP, CACNB4, CCL5, CD33, CD3D, CD93, CD97, CLEC12A, DOK2, FCER1G, FCGR2A, FUCA2, GPR34, GPR84, HCST, HGF, HOMER3, IL2RA, IL2RG, IL3RA, ITGB2, LGALS1, LRG1, LY86, MGAT4A, P2RY5, PRSS21, PTH2R, RNASE2, SLC43A3, SUCNR1, TIMP1, TNF, TNFRSF4, TNFSF13B, TYROBP and VNN1; cell cycle-related genes consisting of ZWINT, NEK6 and TXNL4B; an apoptosis-related gene consisting of BIK; signaling-related genes consisting of AK5, ARHGAP18, FYB, HCK, LPXN, PDE9A, PDK1, PRKCD, RAB20, RAB8A and RABIF; transcription factor genes consisting of WT1 and HLX; and other genes consisting of CYBB, CTSC and NCF4.

Marker Genes (3):

cell membrane- or extracellularly-localized genes consisting of ALOX5AP, CACNB4, CCL5, CD33, CD3D, CD93, CD97, CLEC12A, DOK2, FCGR2A, GPR84, HCST, HOMER3, ITGB2, LGALS1, LRG1, PTH2R, RNASE2, TNF, TNFSF13B, TYROBP and VNN1; a cell cycle-related gene consisting of NEK6; an apoptosis-related gene consisting of BIK; signaling-related genes consisting of AK5, FYB, HCK, LPXN, PDE9A, PDK1, PRKCD and RAB20; a transcription factor gene consisting of WT1; and other genes consisting of CTSC and NCF4.

Although the subject in the test method of the present invention is not particularly limited, as far as it is a mammal, including a human, a human suspected of suffering the initial onset or a recurrence of leukemia is preferred.

The biological sample to be measured by the test method of the present invention is not particularly limited, as far as it can be collected from a mammal, preferably from a human; examples include humoral samples such as blood, bone marrow fluid, and lymph fluid, and solid samples such as lymph nodes, blood vessels, bone marrow, brain, spleen, and skin.

In the test method of the present invention, the expression level of a marker gene is measured for a transcription product or translation product of the gene as an analyte. When the transcription product is the analyte, RNA can be isolated from the biological sample by a conventional method. Ordinary methods for RNA extraction are well known in the relevant technical field, and are disclosed in standard textbooks of molecular biology, including Ausubel et al., Current Protocols of Molecular Biology, John Wiley and Sons (1997) and the like. Specifically, isolation of RNA can be achieved using purification kits, buffer solution sets, and proteases obtained from their manufacturers, such as Qiagen, as directed by the manufacturers.

The method of measuring the expression level of a marker gene for a transcription product as an analyte is not particularly limited; available methods include Northern blotting and in situ hybridization (Parker & Barnes, Methods in Molecular Biology 106: 247-283 (1999)); RNase protection assay (Hod, Biotechniques 13: 852-854 (1992)); reverse transcription polymerase chain reaction (RT-PCR) (Weis et al., Trends in Genetics 8: 263-264 (1992)); realtime quantitative RT-PCR (Held et al., Genome Research 6: 986-994 (1996)); microarray analysis and the like. Microarray analysis can be performed using, the Affymetrix GeneChip technique, the microarray technique of Agilent Technologies or the microarray technique of Incyte with a commercially available apparatus, as directed by the manufacturer. Details of realtime quantitative RT-PCR are described in Examples below. Examples of the base sequences of primers and probes that are suitably used for realtime quantitative RT-PCR are listed in Table 3 and the sequence listing.

When the translation product of a marker gene is the analyte, protein can be isolated from the biological sample according to a conventional method. Ordinary methods for protein extraction are well known in the relevant technical field, and are disclosed in standard textbooks of molecular biology, including Ausubel et al., Current Protocols of Molecular Biology, John Wiley and Sons (1997) and the like. Isolation of protein can be achieved using purification kits, buffer solution sets, and protease inhibitors obtained from their manufacturers, as directed by the manufacturers.

The method of measuring the expression level of a marker gene for a translation product as an analyte is not particularly limited; available methods include the immunohistochemical method, the proteomics method and the like. The immunohistochemical method comprises detecting the expression using an antibody specific for each marker gene product. Protocols and kits for the immunohistochemical method are well known in the relevant technical field, and are commercially available. The proteomics method comprises examining overall changes in protein expression in a certain sample. The proteomics method generally comprises the following steps: (1) separation of various proteins in the sample by 2-D gel electrophoresis (2-D PAGE), (2) identification of the various proteins recovered from this gel by, for example, mass analysis or N-terminal sequencing, and (3) data analysis using bioinformatics. The proteomics method is a useful method for supplementing other gene expression profiling methods, and can be used alone, or in combination with another method, to detect products of marker genes of the present invention. When a cell surface marker is the target, measuring method using flow cytometry is possible.

(2) Step of comparing the expression levels obtained in the measuring step with a reference value

When the results of measurements of the expression levels of 2 to 218 kinds of marker genes in a biological sample show that the expression levels of 2 kinds or more thereof are significantly higher than reference values (gene expression differs about 2 fold or more, preferably about 4 fold or more, more preferably about 6 fold or more, most preferably about 10 fold or more), the possible presence of a leukemic stem cell in the sample or the subject's body is suggested. Here, useful reference values include comparator values such as mean expression levels for healthy persons and mean levels for the subject before onset. The suggestion of the possible presence of leukemic stem cell leads to prediction of the initial onset or a recurrence of leukemia in the subject. It is preferable that the presence or absence of the initial onset or a recurrence of leukemia be checked by another test.

In the test method of the present invention, when the results of measurements of the expression levels of 2 to 218 kinds of marker genes in a biological sample show that the expression levels of 2 kinds or more thereof are significantly higher than reference values (gene expression differs about 2 fold or more, preferably about 4 fold or more, more preferably about 6 fold or more, most preferably about 10 fold or more), the possible presence of a leukemic stem cell in the sample or the body of the source from which the sample has been collected is suggested. Here, useful reference values include comparator values such as mean expression levels for healthy persons and mean expression level for the subject before onset. In this case, the suggestion of the possible presence of a leukemic stem cell leads to prediction that the treatment is not completely effective on the cancer in the leukemia patient. Conversely, when the expression levels of the aforementioned 2 kinds or more are significantly lower (for example, substantially zero), it can be predicted that leukemic stem cells are absent in the sample. In this case, it is thought that the treatment of leukemia eliminated leukemic stem cells and is effective. Furthermore, it is preferable that the test method be combined with other examinations to achieve multi-angle confirmation of a therapeutic effect on leukemia.

As stated above, by applying the test method of the present invention, it is possible to detect leukemic stem cells in a living organism before leukemia occurs initially or recurs, and predict the onset. Alternatively, it is also possible to detect the onset of leukemia in the initial stage and lead to early treatment of cancer patients. Furthermore, it is also possible to evaluate the therapeutic effect on leukemia patients with the presence or absence of leukemic stem cells as an index.

### (Therapeutic Agent)

The present invention also provides a therapeutic agent for acute myeloid leukemia that targets leukemic stem cells, comprising as an active ingredient a substance capable of suppressing the expression of a leukemic stem cell marker gene or a substance capable of suppressing the activity of a translation product of the gene.

Molecular targets for the therapeutic agent of the present invention are the above-described leukemic stem cell marker genes, and any marker may be selected according to the purpose of treatment. When the therapeutic agent of the present invention targets stem cells, out of leukemic stem cells, that are present in bone marrow niches, are in the stationary phase of cell cycle, and are resistant to anticancer agents, it is recommended that a substance capable of suppressing the expression of genes selected from the group consisting of AK5, BIK, DOK2, FCGR2A, IL2RA, LRG1, SUCNR1 and WT1 (hereinafter also referred to as marker genes (4)) or a substance capable of suppressing the activity of a translation product of the gene be selected. By selecting 2 to 8 (preferably 2 to 5) out of the eight genes constituting the marker genes (4) and using them as molecular targets, it is highly likely possible to exterminate leukemic stem cells of a large number of patients. Therefore, at least one active ingredient is contained in the therapeutic agent of the present invention, and it is preferable that two or more be combined according to the purpose of treatment. Two or more active ingredients may be contained in a single pharmaceutical preparation, or may be contained in separate pharmaceutical preparations.

Described below are active ingredients.

Substances capable of suppressing the expression of a leukemic stem cell marker gene include, for example, antisense nucleic acids, RNAi-inducible nucleic acids and the like.

Substances capable of suppressing the activity of a translation product of a leukemic stem cell marker gene include, for example, aptamers, antibodies and the like. The substance may be an inhibitory substance that acts directly or indirectly on each marker.

Described below are active ingredients of the therapeutic agent of the present invention.

### 1. Antisense Nucleic Acid

The kind of the antisense nucleic acid may be DNA or RNA, or a DNA/RNA chimera. The antisense nucleic acid may be one having a natural type phosphoric acid diester bond, or a modified nucleotide such as of the thiophosphate type (P═O in phosphate linkage replaced with P═S), 2′-O-methyl type and the like, which are stable to decomposing enzymes. Other important factors for the designing of antisense nucleic acids include increases in water-solubility and cell membrane permeability and the like; these can also be cleared by choosing appropriate dosage forms such as those using liposome or microspheres. The length of the antisense nucleic acid is not particularly limited, as far as the antisense nucleic acid is capable of specifically hybridizing with the transcription product; the antisense nucleic acid may be a sequence comprising about 15 nucleotides for the shortest, or comprising a sequence complementary to the entire sequence of the transcription product for the longest. Taking into account the issues of the ease of synthesis, antigenicity and the like, oligonucleotides consisting of, for example, about 15 or more nucleotides, preferably about 15 to about 100 nucleotides, more preferably about 18 to about 50 nucleotides, can be mentioned as examples. Furthermore, the antisense nucleic acid may be one that not only hybridizes with the transcription product to inhibit the translation, but also is capable of binding to a double-stranded DNA to form a triple strand (triplex) to inhibit the transcription into mRNA.

### 2. RNAi-Inducible Nucleic Acid

An RNAi-inducible nucleic acid refers to a polynucleotide, preferably an RNA, capable of inducing the RNA interference (RNAi) effect when introduced into cells. The RNAi effect refers to the phenomenon in which a double-stranded RNA comprising the same nucleic acid sequence as that of mRNA, or a partial sequence thereof, suppresses the expression of the mRNA. To obtain the RNAi effect, it is preferable to use, for example, a double-stranded RNA having the same nucleic acid sequence as that of the target mRNA comprising at least 19 continuous bases (or a partial sequence thereof). The double-stranded structure may be configured by different strands, or may be a double strand conferred by a stem-loop structure of one RNA. Examples of RNAi-inducing nucleic acids include siRNAs, miRNAs and the like, with preference given to siRNAs. The siRNA is not particularly limited, as far as it can induce an RNAi, and the siRNA can be, for example, 19 to 27 bases long, preferably 21 to 25 bases long.

### 3. Aptamer

An aptamer refers to a polynucleotide having a binding activity (or inhibitory activity) on a specified target molecule. An aptamer is an RNA, a DNA, a modified nucleotide or a mixture thereof. The aptamer can be in a linear or circular form. The length of the aptamer is not particularly limited, and is normally about 16 to about 200 nucleotides; for example, the length is about 100 nucleotides or less, preferably about 50 nucleotides or less, and more preferably about 40 nucleotides or less. The length of the aptamer may be, for example, about 18, about 20, about 25 or about 30, nucleotides or more. The aptamer, for increasing the bindability, stability, drug delivering quality and the like, may be one wherein a sugar residue (e.g., ribose) of each nucleotide is modified. Examples of portions of the sugar residue where it is modified include ones wherein the oxygen atom at the 2′-position, 3′-position and/or 4′-position of the sugar residue is replaced with another atom and the like. Examples of types of modifications include fluorination, O-alkylation, O-allylation, S-alkylation, S-allylation and amination (see, e.g., Sproat et al., (1991) Nucle. Acid. Res. 19, 733-738; Cotton et al., (1991) Nucl. Acid. Res. 19, 2629-2635). The aptamer may also be one wherein a purine or pyrimidine is altered. Examples of such alterations include alteration of the 5-position pyrimidine, alteration of the 8-position purine, alteration with an exocyclic amine, substitution with 4-thiouridine, and substitution with 5-bromo or 5-iodo-uracil. The phosphate group contained in the aptamer of the present invention may be altered to make it resistant to nucleases and hydrolysis. For example, the phosphate group may be substituted with a thioate, a dithioate or an amidate. An aptamer can be prepared according to available reports (for example, Ellington et al., (1990) Nature, 346, 818-822; Tuerk et al., (1990) Science, 249, 505-510).

### 4. Antibody

The antibody may be a polyclonal antibody (antiserum) or a monoclonal antibody, and can be prepared by a commonly known immunological technique. Although the monoclonal antibody may be of any isotype, IgG, IgM, IgA, IgD, IgE, or the like, IgG or IgM is preferable.

For example, the polyclonal antibody can be acquired by subcutaneously or intraperitoneally administering the above-described antigen (as required, may be prepared as a complex crosslinked to a carrier protein such as bovine serum albumin or KLH (Keyhole Limpet Hemocyanin)), along with a commercially available adjuvant (for example, Freund's complete or incomplete adjuvant), to an animal about 2 to 4 times at intervals of 2 to 3 weeks (the antibody titer of partially drawn serum has been determined by a known antigen-antibody reaction and its elevation has been confirmed in advance), collecting whole blood about 3 to 10 days after final immunization, and purifying the antiserum. Animals to receive the antigen include mammals such as rats, mice, rabbits, goat, guinea pigs, and hamsters.

The monoclonal antibody can also be prepared by cell fusion. For example, the above-described antigen, along with a commercially available adjuvant, is subcutaneously or intraperitoneally administered to a mouse 2 to 4 times, and 3 days after final administration, the spleen or lymph nodes are collected, and leukocytes are collected. These leukocytes and myeloma cells (for example, NS-1, P3X63Ag8 and the like) are cell-fused to obtain a hybridoma that produces a monoclonal antibody against the factor. This cell fusion may be performed by the PEG method or the voltage pulse method. A hybridoma that produces the desired monoclonal antibody can be selected by detecting an antibody that binds specifically to the antigen in the culture supernatant, using a widely known EIA or RIA method and the like. Cultivation of the hybridoma that produces the monoclonal antibody can be performed in vitro, or in vivo such as in ascitic fluid of a mouse or rat, preferably a mouse, and the antibody can be acquired from the culture supernatant of the hybridoma or the ascitic fluid of the animal.

The antibody may be a chimeric antibody, a humanized antibody or a human antibody.

A chimeric antibody means a monoclonal antibody derived from immunoglobulins of animal species having mutually different variable regions and constant regions. For example, the chimeric antibody can be a mouse/human chimeric monoclonal antibody whose variable region is a variable region derived from a mouse immunoglobulin, and whose constant region is a constant region derived from a human immunoglobulin. The constant region derived from a human immunoglobulin has an amino acid sequence unique depending on the isotype, IgG, IgM, IgA, IgD, IgE or the like, and the constant region of a recombinant chimeric monoclonal antibody in the present invention may be the constant region of a human immunoglobulin belonging to any isotype. The constant region of human IgG is preferable.

A chimeric antibody can be prepared by a method known per se. For example, a mouse/human chimeric monoclonal antibody can be prepared according to available reports (e.g., Jikken Igaku (extra issue), Vol. 6, No. 10, 1988 and JP-B-HEI-3-73280). In detail, a mouse/human chimeric monoclonal antibody can be prepared by inserting the C_{H} gene acquired from the DNA that encodes a human immunoglobulin (C gene that encodes H chain constant region) downstream of the active V_{H} gene acquired from the DNA that encodes a mouse monoclonal antibody, isolated from a hybridoma that produces the mouse monoclonal antibody (rearranged VDJ gene that encodes H chain variable region), and inserting the C_{L} gene acquired from the DNA that encodes a human immunoglobulin (C gene that encodes L chain constant region) downstream of the active V_{L} gene acquired from the DNA that encodes a mouse monoclonal antibody, isolated from the hybridoma (rearranged VJ gene that encodes L chain variable region), into one or separate expression vectors in a way that allows the expression of each gene, transforming a host cell with the expression vector, and culturing the transformant cell.

A humanized antibody means a monoclonal antibody prepared by a gene engineering technique, for example, a human type monoclonal antibody wherein some or all of the complementarity-determining regions of the ultra-variable region thereof are derived from a mouse monoclonal antibody, and the framework region of the variable region thereof and the constant region thereof are derived from a human immunoglobulin. The complementarity-determining regions of the ultra-variable region are three regions that are present in the ultra-variable region in the variable region of the antibody, and that complementarily bind directly to the antigen (complementarity-determining regions; CDR1, CDR2, CDR3), and the framework regions of the variable region are four relatively highly conserved regions interposing the front and back of the three complementarity-determining regions (frameworks; FR1, FR2, FR3, FR4). Hence, a humanized antibody means, for example, a monoclonal antibody wherein all regions other than some or all of the complementarity-determining regions of the ultra-variable region of a mouse monoclonal antibody are replaced with corresponding regions of a human immunoglobulin.

A humanized antibody can be prepared by a method known per se. For example, a recombinant humanized antibody derived from a mouse monoclonal antibody can be prepared according to available reports (e.g., Japanese Patent Application Kohyo Publication No. HEI-4-506458 and JP-A-SHO-62-296890). In detail, from a hybridoma that produces a mouse monoclonal antibody, at least one mouse H chain CDR gene and at least one mouse L chain CDR gene corresponding to the mouse H chain CDR gene are isolated, and from a human immunoglobulin gene, the human H chain gene that encodes all regions other than the human H chain CDR corresponding to the mouse H chain CDR and the human L chain gene that encodes all regions other than the human L chain CDR corresponding to the mouse L chain CDR are isolated. The mouse H chain CDR gene and human H chain gene isolated are introduced into an appropriate expression vector expressibly; likewise, the mouse L chain CDR gene and the human L chain gene are introduced into another appropriate expression vector expressively. Alternatively, the mouse H chain CDR gene/human H chain gene and the mouse L chain CDR gene/human L chain gene can be introduced into the same expression vector expressively. By transforming a host cell with the expression vector thus prepared, it is possible to obtain a cell that produces a humanized antibody, and by culturing the cell, the desired humanized antibody can be obtained from the culture supernatant.

A human antibody means an antibody wherein all regions comprising the variable regions and constant regions of the H chain and L chain constituting an immunoglobulin are derived from the gene that encodes a human immunoglobulin.

A human antibody can be prepared by a method known per se. For example, a human antibody can be produced by immunologically sensitizing with an antigen a transgenic animal prepared by incorporating at least a human immunoglobulin gene into a gene locus of a non-human mammal such as a mouse, in the same way as the above-described method of preparing a polyclonal antibody or a monoclonal antibody. For example, a transgenic mouse that produces a human antibody can be prepared according to available reports (Nature Genetics, Vol. 15, p. 146-156, 1997; Nature Genetics, Vol. 7, p. 13-21, 1994; Japanese Patent Application Kohyo Publication No. HEI-4-504365; International Patent Application Publication WO94/25585; Nature, Vol. 368, p. 856-859, 1994; and Japanese Patent Application Kohyo Publication No. HEI-6-500233).

The antibody may be a part of the above-mentioned antibody (e.g., monoclonal antibody). The antibody may be a fragment such as F(ab′)₂, Fab′, Fab, Fv and the like, a conjugate molecule prepared by genetic engineering such as scFv, scFv-Fc, minibody, diabody and the like, or a derivative thereof, which is modified by a molecule and the like having a protein stabilizing action such as polyethylene glycol (PEG) and the like, and the like.

The above-described antibody may be in the form of an immunoconjugate bound with various anticancer substances and the like by a conventional method. In this case, the antibody functions as a drug delivery system for delivering an anticancer agent to LSCs. Anticancer substances to be combined include, but are not limited to, cisplatin, carboplatin, cyclophosphamide, melphalan, carmusulin, methotrexate, 5-fluorouracil, cytarabine (AraC), mercaptopurine, daunorubicin, idarubicin, mitoxantrone, thioguanine, azacitidine, amsacrine, doxorubicin, tretinoin, allopurinol, prednisone (prednisolone), epirubicin, vinblastine, vincristine, dactinomycin (actinomycin), mitomycin C, taxol, L-asparaginase, etoposide, colchicine, deferoxamine mesylate, camptothecin and the like. Furthermore, the antibody may be an immunoconjugate with a radionuclide, toxin and the like.

The agent of the present invention can comprise, in addition to a substance capable of suppressing the expression of a leukemic stem cell marker gene or the activity of a translation product of the gene, an optionally chosen carrier, for example, a pharmaceutically acceptable carrier. Examples of pharmaceutically acceptable carriers include, but are not limited to, excipients such as sucrose, starch, mannitol, sorbitol, lactose, glucose, cellulose, talc, calcium phosphate and calcium carbonate; binders such as cellulose, methylcellulose, hydroxypropylcellulose, polypropylpyrrolidone, gelatin, acacia, polyethylene glycol, sucrose and starch; disintegrants such as starch, carboxymethylcellulose, hydroxypropyl starch, sodium-glycol-starch, sodium hydrogen carbonate, calcium phosphate and calcium citrate; lubricants such as magnesium stearate, Aerosil, talc and sodium lauryl sulfate; flavoring agents such as citric acid, menthol, glycyrrhizin ammonium salt, glycine and orange powder; preservatives such as sodium benzoate, sodium hydrogen sulfite, methyl paraben and propyl paraben; stabilizers such as citric acid, sodium citrate and acetic acid; suspending agents such as methylcellulose, polyvinylpyrrolidone and aluminum stearate; dispersing agents such as surfactants; diluents such as water, physiological saline and orange juice; base waxes such as cacao butter, polyethylene glycol and refined kerosene; and the like.

Preparations suitable for oral administration are liquids prepared by dissolving an effective amount of a substance in a diluent such as water or physiological saline, capsules, sachets or tablets containing an effective amount of a substance in the form of solids or granules, suspensions prepared by suspending an effective amount of a substance in an appropriate dispersant, emulsions prepared by dispersing and emulsifying a solution of an effective amount of a substance in an appropriate dispersant, or powders, granules and the like.

Preparations suitable for parenteral administration (e.g., intravenous injection, subcutaneous injection, intramuscular injection, topical injection and the like) are aqueous and non-aqueous isotonic sterile injectable liquids, which may contain an antioxidant, a buffer solution, a bacteriostatic agent, an isotonizing agent and the like. Aqueous and non-aqueous sterile suspensions can also be mentioned, which may contain a suspending agent, a solubilizer, a thickening agent, a stabilizer, an antiseptic and the like. These preparations can be enclosed in containers such as ampoules and vials for unit dosage or a plurality of dosages. It is also possible to freeze-dry the active ingredient and a pharmaceutically acceptable carrier, and store the preparation in a state that may be dissolved or suspended in an appropriate sterile vehicle just before use.

Although the dosage of the agent of the present invention varies depending on the activity and choice of active ingredient, the mode of administration (e.g., oral, parenteral), the seriousness of disease, the animal species which is the subject of administration, the drug tolerance, body weight and age of the subject of administration, and the like, and cannot be generalized, it is normally about 0.001 mg to about 5.0 g as the amount of active ingredient per day for an adult.

The subject of administration of the agent of the present invention is not particularly limited, as far as it is an animal species having a hematopoietic tissue (bone marrow), and possibly contracting acute myeloid leukemia, and it is preferably a mammal, more preferably a human.

### (Method of Production)

The present invention also provides a method for producing a sample containing hematopoietic cells for autologous transplantation or allogeneic transplantation for a patient with acute myeloid leukemia. The production method of the present invention comprises,

a) a step of collecting a sample containing hematopoietic cells from the patient or a donor,
b) a step of bringing the collected sample into contact with at least one kind of substance that recognizes a translation product of a leukemic stem cell marker gene, and
c) a step of sorting cells to which the above-described substance has been bound, and obtaining the sample from which leukemic stem cells have been purged. Accordingly, the present invention makes it possible to substantially remove leukemic stem cells from a sample containing hematopoietic cells for autologous transplantation or allogeneic transplantation, and provide a sample for transplantation without the fear of recurrences.

The leukemic stem cell marker genes are as mentioned above; for the purpose of purging, however, it is preferred to target at least one kind of cell surface marker gene selected from among the following set of genes:

ADFP, ALOX5AP, CACNB4, CD33, CD3D, CD93, CD97, CLEC12A, DOK2, FCER1G, FCGR2A, GPR34, GPR84, HCST, HOMER3, IL2RA, IL2RG, IL3RA, ITGB2, LY86, P2RY5, PTH2R, SUCNR1, TNFRSF4, TYROBP and VNN1.
a) Step of collecting a sample containing hematopoietic cells from a patient with acute myeloid leukemia or a donor

Sample collection is normally achieved by bone marrow aspiration or peripheral blood collection. Bone marrow aspiration is performed on the sternum or ilium on the basis of, for example, the method described in S. E. Haynesworth et al., Bone, 13, 81 (1992) and the like. Specifically, the skin surface of the portion for aspirating the bone marrow is disinfected, and topical anesthesia is performed. The subperiosteal region, in particular, is anesthetized sufficiently. The inner cylinder of the puncture needle is removed, a 10 mL syringe containing 5000 units of heparin is attached, and the required amount of bone marrow fluid is quickly aspirated. On average, 10 mL to 20 mL of bone marrow fluid is aspirated. The puncture needle is removed, and astriction is performed for about 10 minutes. The bone marrow fluid acquired is centrifuged at 1,000×g, and bone marrow cells are recovered, after which the bone marrow cells are washed with PBS (Phosphate Buffered Saline). After the washing step is repeated several times, a sample containing hematopoietic cells can be obtained.

In the case of peripheral blood, collection is performed from a vein. Specifically, the skin surface of the portion for peripheral blood collection is disinfected. The inner cylinder of the injection needle is removed, a 10 mL syringe containing 5000 units of heparin is attached, and the required amount of peripheral blood is quickly aspirated. On average, 10 mL to 20 mL of peripheral blood is aspirated. The injection needle is removed, and astriction is performed for about 10 minutes. The peripheral blood acquired is centrifuged at 1,000×g, and peripheral blood cells are recovered, after which the peripheral blood cells are washed with PBS (Phosphate Buffered Saline). After the washing step is repeated several times, a sample containing hematopoietic cells can be obtained.

b) Step of bringing the collected sample into contact with a substance that recognizes a translation product of at least one kind of leukemic stem cell marker gene

The substances that recognize a translation product of the marker genes for use in this step include antibodies described above, with particular preference given to antibodies against at least one kind of cell surface marker selected from among ADFP, ALOX5AP, CACNB4, CD33, CD3D, CD93, CD97, CLEC12A, DOK2, FCER1G, FCGR2A, GPR34, GPR84, HCST, HOMER3, IL2RA, IL2RG, IL3RA, ITGB2, LY86, P2RY5, PTH2R, SUCNR1, TNFRSF4, TYROBP and VNN1. Preferably, the antibodies are fluorescently labeled, and preferable fluorescent dyes used for the labeling are fluorescent substances commonly used for flow cytometry. Specific examples of fluorescent dyes include FITC (fluorescein isothiocyanate), PE (phycoerythrin), PerCP (peridinin-chlorophyll-protein), PerCP-Cy5.5, PE-Cy5, PE-Cy7, PE-TR (PE-Texas Red), APC (allophycocyanin), APC-Cy7 and the like. Conditions for the contacting are not particularly limited, as far as a contact between the above-mentioned cell surface marker (antigen) and the antibody can be achieved.

c) Step of sorting cells to which the above-described substance has bound, and obtaining the sample from which leukemic stem cells have been purged

In this step, cell sorting can easily be accomplished by combining with flow cytometry. The sample in contact with a fluorescently labeled antibody is set to a flow cytometer, and the cells bound to the antibody are sorted; leukemic stem cells can be separated from the sample.

The thus-obtained LSC-purged sample can be used for the treatment of AML patients, without the fear of recurrences, as the LSCs have been efficiently eliminated, whereas HSCs have been concentrated escaping elimination.

### EXAMPLES

The present invention is hereinafter described in detail by means of the following Examples, by which, however, the invention is not limited in any way.

### Human Samples

All experiments were conducted with the approval of the Institutional Review Board for Human Research of the RIKEN Research Center for Allergy and Immunology. Leukemia cells derived from AML patients were collected with informed consent in writing. CB (cord blood) derived from healthy donors, along with informed consent in writing, was collected by the Tokyo Cord Blood Bank. BMMNCs (bone marrow mononuclear cells) derived from healthy donors were obtained from Cambrex (Walkerville, Md.). BMMNCs and CBMNCs (cord blood mononuclear cells) derived from AML patients were isolated using density gradient centrifugation.

### FACS and Flow Cytometric Analysis

For fluorescence-activated cell sorting (FACS), BMMNC cells from AML patients were labeled with fluorescent dye-coupled mouse anti-hCD3, anti-hCD4, anti-hCD8, anti-hCD34 and anti-hCD38 monoclonal antibodies (BD Biosciences, San Jose, Calif.), and recipient BMMNC cells were labeled with mouse anti-hCD45, anti-hCD34 and anti-hCD38 monoclonal antibodies (BD Biosciences); the cells were sorted using FACSAria (BD Biosciences). Doublets were eliminated via analyzing FSC/SSC height and FSC/SSC width. After the sorting, the purity of hCD34+hCD38− and hCD34+ cells was higher than 98%. For flow cytometric analysis, BMMNCs of AML patients, recipient peripheral blood or recipient BM was labeled with the above-described fluorescent dye-coupled mouse anti-hCD3, anti-hCD4, anti-hCD8, anti-hCD34 and anti-hCD38 monoclonal antibodies or mouse anti-hCD45, anti-hCD34 and anti-hCD38 monoclonal antibodies.

### Microarray Analysis

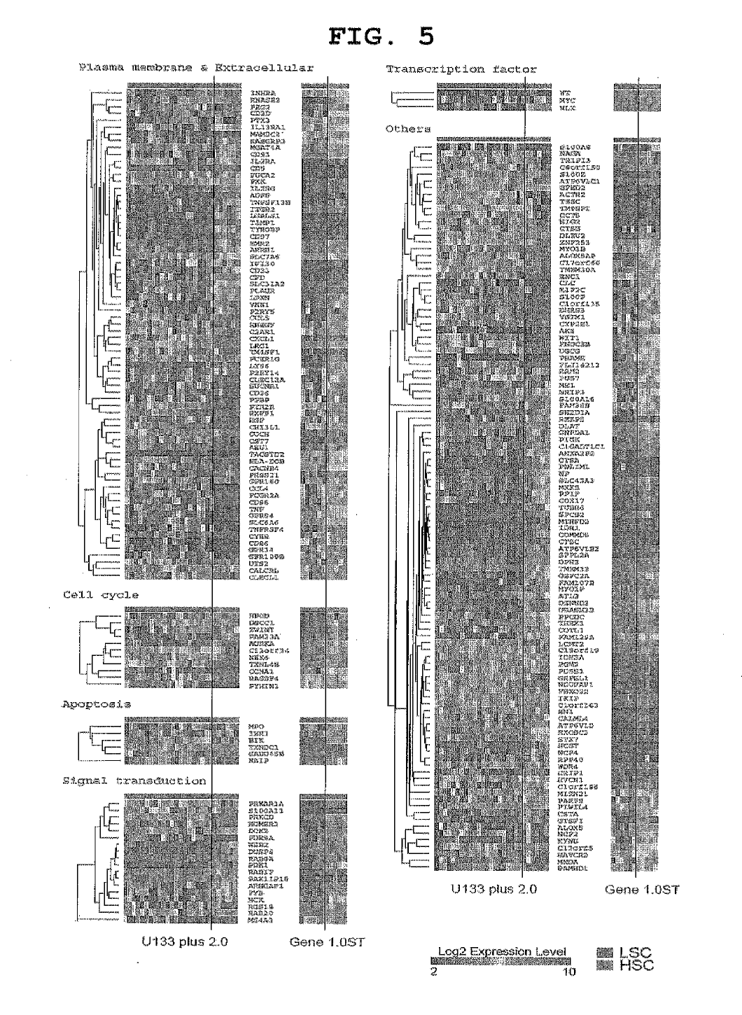

Total RNA was extracted using TRIzol Reagent (Invitrogen), and the integrity of the RNA was then assessed with Agilent Bioanalyzer. Biotinylated cRNAs were synthesized using Two-Cycle Target Labeling Kit (Affymetrix) for Human Genome U133 plus 2.0 GeneChip (Affymetrix). For Human Gene 1.0ST GeneChip (Affymetrix), a first round of cDNA synthesis and cRNA amplification were performed using MessageAmp Premier RNA Amplification Kit (Applied Biosystems), and a subsequent second round of cDNA synthesis, biotinylation and fragmentation were performed using WT cDNA Synthesis and Terminal Labeling kits (Affymetrix). Hybridization, washing, staining and scanning were performed according to the manufacturers' instruction. Firstly, the microarray data for each platform was separately analyzed using Bioconductor package (http://www.bioconductor.org/). The signal intensities of probe sets on the microarray platforms were normalized with GC-RMA program (Zhijin et al., J. Am. Stat. Assoc., 99, 909-917, 2004). For each platform, the normalized data was analyzed with RankProd program (Hong et al., Bioinformatics, 22, 2825-2827, 2006) to select genes differentially expressed between LSCs and HSCs with the cutoff p value of 0.01 and the false-positive estimation of 0.05%. When a significantly higher level of expression was observed in LSC than in HSC commonly in both the microarray platforms, the gene was selected as a significant candidate LSC marker gene (FIG. 5, Table 1). In addition, the gene IL2RA, which gave a high hit rate for Human Gene 1.0ST GeneChip, and provided favorable results in the protein level analysis, was also selected as a candidate marker gene, since it is expressed in stem cells resistant to anticancer drugs as described below (Table 1). The localization and the biological function of the candidates were annotated based on information from Ingenuity Pathway Analysis Database (Ingenuity Systems) and Gene Ontology Annotation Database (http://www.ebi.ac.uk/GOA/).

Quantitative PCR (qPCR) Analysis

Ten ng of total RNA from HSCs or LSCs was subjected to cDNA amplification using WT-Ovation RNA Amplification System (Nugen). The cDNA products were diluted 1:7.5 in TE, and 1 μl of the dilution products was used per 25 μl of qPCR reaction. The sequences of doubly-labeled fluorescent probes and gene specific primers (Sigma-Aldrich) were listed in Table 3. PCR reactions were performed using LightCycler 480 (Roche Applied Science) with Platinum Quantitative PCR SuperMix-UDG (Invitrogen). The abundance of the respective transcripts was calculated by the standard curve method (Methods, 25, 386-401, 2001). When any of Kruskal-Wallis, Wilcoxon-Mann-Whitney and Student's t-test in Kaleida Graph software package showed P<0.05, it was determined there is a significant difference in the expression level between LSC and HSC.

### Animals

NOD.Cg-Prkdc^{scid}Il2rg^{tmlWjl}/Sz (NOD/SCID/IL2rgγ^{null}) mice were developed at The Jackson Laboratory (Bar Harbor, Me.) by backcrossing a complete null mutation at the Il2rg locus onto the NOD.Cg-Prkdc^{scid} (NOD/SCID) strain (Shultz, L. D. et al. Multiple defects in innate and adaptive immunologic function in NOD/LtSz-scid mice. J Immunol 154, 180-191 (1995)). Mice were bred and maintained under defined flora with irradiated food and acidified water at the animal facility of RIKEN and at The Jackson Laboratory according to guidelines established by the Institutional Animal Committees at the respective institutions.

### Heterologous Transplantation

Newborn (within 2 days of birth) NOD/SCID/IL2rg^{null} mice received 150 cGy of total body irradiation using a ¹³⁷Cs-source irradiator, followed by intravenous injection of AML cells within two hours. The recipients were subjected to blood sampling from retro-orbital every 3-4 weeks, and human AML transplantation chimerism in peripheral blood was assessed.

### Immunofluorescent Labeling and Imaging

Para-formaldehyde-fixed decalcified paraffin-embedded sections were prepared from a femoral bone of a primary AML transplantation recipient. The primary antibodies used for labeling were a mouse anti-human CD45 monoclonal antibody (DAKO, Denmark) and a rabbit anti-CD32 monoclonal antibody (Abcam, UK). Laser scanning confocal imaging was obtained using Zeiss LSM Exciter and LSM 710 (Carl Zeiss).

### Immunofluorescent Labeling and Imaging (2)

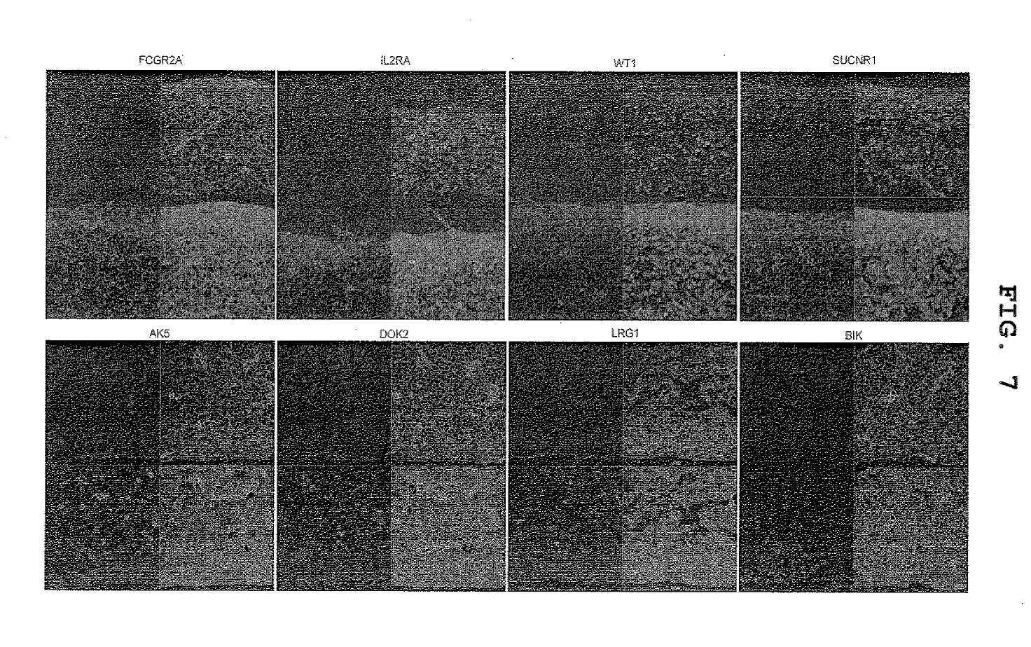

Para-formaldehyde-fixed decalcified paraffin-embedded sections were prepared from a femoral bone of a recipient of transplantation of primary AML treated with an anticancer agent, and stained with antibodies against DAPI (nuclear staining: blue); various markers (FCGR2A, AK5, DOK2, LRG1, BIK, IL2RA, WT1, SUCNR1: red); and stationary cell markers (green: CD34 (FCGR2A, AK5, DOK2, LRG1, BIK) or Ki67 (IL2RA, WT1, SUCNR1). Laser scanning confocal imaging was obtained using Zeiss LSM Exciter and LSM 710 (Carl Zeiss) (FIG. 7).

TABLE 2
List of genes whose transcription product is
expressed in larger amounts in AML CD3+CD38− LSCs than in
normal CD34+CD38− HSCs

| | | | | | | | | | Number |
|---|---|---|---|---|---|---|---|---|---|
| | | | | | | | | | of LSC |
| | | | | | | | | | samples |
| | | | | | | | | | showing |
| | | | | | | | | | a higher |
| | | | | | | | | | expression |
| | | | | | | | | Ratio of | than any |
| | | | | | Statistics | | | median | HSC |
| | | | | | | Wilcoxon- | | values | samples |
| | | | | | | Mann- | Kruskal- | (LSC/ | (Maxi- |
| GeneID | Gene name | Location | Function | Process | T-test | Whiteny | Wallis | HSC) | mum: 5) |
| 123 | ADFP | cell membrane | | | 0.011 | 0.032 | 0.027 | 5.5 | 4 |
| 26289 | AK5 | cytoplasm | signaling molecule | | 0.014 | 0.018 | 0.013 | >10000 | 5 |
| 241 | ALOX5AP | cell membrane | | | 0.125 | 0.016 | 0.014 | 33.8 | 5 |
| 93663 | ARHGAP18 | unknown | signaling molecule | | 0.033 | 0.063 | 0.050 | 2.3 | 4 |
| 688 | BIK | cytoplasm | | apoptosis | 0.087 | 0.016 | 0.014 | 129.4 | 5 |
| 785 | CACNB4 | cell membrane | | | 0.009 | 0.016 | 0.014 | 50.4 | 5 |
| 6852 | CCL5 | extracellular space | cytokine | immunity, cell adhesion | 0.018 | 0.016 | 0.014 | 58.4 | 5 |
| 945 | CD33 | cell membrane | signaling molecule | cell adhesion | 0.002 | 0.016 | 0.014 | 8.0 | 5 |
| 915 | CD3D | cell membrane | transmembranous | | 0.086 | 0.015 | 0.011 | >10000 | 5 |
| | | | receptor | | | | | | |
| 22918 | CD93 | cell membrane | | cell adhesion | 0.018 | 0.016 | 0.014 | 27.1 | 5 |
| 976 | CD97 | cell membrane | transmembranous | immunity, cell adhesion | 0.014 | 0.016 | 0.014 | 5.7 | 5 |
| | | | receptor | | | | | | |
| 160864 | CLEC12A | cell membrane | | | 0.049 | 0.016 | 0.014 | 180.2 | 5 |
| 1075 | CTSC | cytoplasm | | immunity | <0.001 | 0.016 | 0.014 | 6.6 | 5 |
| 1536 | CYBB | cytoplasm | | immunity | 0.107 | 0.036 | 0.027 | 639.5 | 4 |
| 9046 | DOK2 | cell membrane | signaling molecule | | 0.080 | 0.019 | 0.014 | 81.5 | 5 |
| 2207 | FCER1G | cell membrane | transmembranous | immunity, apoptosic | 0.042 | 0.016 | 0.014 | 24.5 | 4 |
| | | | receptor | | | | | | |
| 2212 | FCGR2A | cell membrane | transmembranous | | 0.347 | 0.016 | 0.014 | 21.1 | 5 |
| | | | receptor | | | | | | |
| 2519 | FUCA2 | extracellular space | | | 0.005 | 0.016 | 0.014 | 2.7 | 5 |
| 2533 | FYB | nucleus | signaling molecule | immunity | 0.081 | 0.018 | 0.013 | 5.4 | 5 |
| 2857 | GPR34 | cell membrane | transmembranous | | 0.085 | 0.016 | 0.014 | 4.3 | 5 |
| | | | receptor | | | | | | |
| 58831 | GPR84 | cell membrane | transmembranous | | 0.259 | 0.016 | 0.014 | 3521.9 | 5 |
| | | | receptor | | | | | | |
| 3055 | HCK | cytoplasm | signaling molecule | | 0.031 | 0.016 | 0.014 | 82.6 | 5 |
| 10870 | HCST | cell membrane | | | <0.001 | 0.016 | 0.014 | 17.3 | 5 |
| 3082 | HGF | extracellular space | growth factor | | 0.034 | 0.191 | 0.142 | 27.2 | 4 |
| 3142 | HLX | nucleus | transcriptional | | 0.008 | 0.016 | 0.014 | 2.9 | 5 |
| | | | regulation molecule | | | | | | |
| 9454 | HOMER3 | cell membrane | signaling molecule | | 0.081 | 0.018 | 0.013 | 330.1 | 5 |
| 3561 | IL2RG | cell membrane | transmembranous | immunity | 0.039 | 0.016 | 0.014 | 3.0 | 5 |
| | | | receptor | | | | | | |
| 3563 | IL3RA | cell membrane | transmembranous | | 0.142 | 0.016 | 0.014 | 2.7 | 5 |
| | | | receptor | | | | | | |
| 3689 | ITGB2 | cell membrane | signaling molecule | cell adhesion, apoptosis | 0.016 | 0.016 | 0.014 | 5.6 | 5 |
| 3956 | LGALS1 | extracellular space | | apoptosis | 0.011 | 0.016 | 0.014 | 34.5 | 5 |
| 9404 | LPXN | cytoplasm | signaling molecule | cell adhesion | 0.008 | 0.016 | 0.014 | 5.2 | 5 |
| 116844 | LRG1 | extracellular space | | | 0.023 | 0.016 | 0.014 | 18.8 | 5 |
| 9450 | LT86 | cell membrane | | immunity, apoptosis | 0.166 | 0.065 | 0.049 | 14.8 | 4 |
| 11320 | MGAT4A | extracellular space | | | 0.031 | 0.063 | 0.050 | 2.6 | 4 |
| 4689 | NCF4 | cytoplasm | | immunity | 0.008 | 0.016 | 0.014 | 5.7 | 5 |
| 10783 | NEK6 | nucleus | signaling molecule | cell cycle, apoptosis | 0.007 | 0.016 | 0.014 | 5.3 | 5 |
| 10161 | P2RY5 | cell membrane | transmembranous | | 0.043 | 0.068 | 0.050 | 20.8 | 4 |
| | | | receptor | | | | | | |
| 5152 | PDE9A | cytoplasm | signaling molecule | | 0.140 | 0.016 | 0.014 | 42.0 | 5 |
| 5163 | PDK1 | cytoplasm | signaling molecule | | 0.004 | 0.016 | 0.014 | 11.7 | 5 |
| 5580 | PRKCD | cytoplasm | signaling molecule | | 0.059 | 0.016 | 0.014 | 24.2 | 5 |
| 10942 | PRSS21 | extracellular space | | | 0.023 | 0.191 | 0.142 | 43.3 | 4 |
| 5746 | PTH2R | cell membrane | transmembranous | | 0.188 | 0.019 | 0.014 | 9.3 | 5 |
| | | | receptor | | | | | | |
| 55647 | RAB20 | cytoplasm | signaling molecule | | 0.117 | 0.019 | 0.014 | 157.1 | 5 |
| 4218 | RAB8A | cytoplasm | signaling molecule | | 0.017 | 0.063 | 0.050 | 2.1 | 3 |
| 5877 | RABIF | unknown | signaling molecule | | 0.008 | 0.016 | 0.014 | 3.1 | 5 |
| 6086 | RNASE2 | extracellular space | | | 0.152 | 0.016 | 0.014 | 88.1 | 5 |
| 29015 | SLC43A3 | extracellular space | | | 0.013 | 0.016 | 0.014 | 8.3 | 5 |
| 56670 | SUCNR1 | cell membrane | transmembranous | | 0.075 | 0.032 | 0.027 | 29.9 | 4 |
| | | | receptor | | | | | | |
| 7076 | TIMP1 | extracellular space | | | 0.020 | 0.032 | 0.027 | 3.4 | 4 |
| 7124 | TNF | extracellular space | cytokine | immunity, apoptosis | 0.325 | 0.016 | 0.014 | 2855.3 | 5 |
| 7293 | TNFRSF4 | cell membrane | transmembranous | immunity | 0.327 | 0.034 | 0.025 | >10000 | 4 |
| | | | receptor | | | | | | |
| 10673 | TNFSF13B | extracellular space | cytokine | immunity | 0.017 | 0.016 | 0.014 | 6.1 | 5 |
| 54957 | TXNL4B | nucleus | | cell cycle | 0.045 | 0.032 | 0.027 | 8.4 | 4 |
| 7305 | TYROBP | cell membrane | signaling molecule | | 0.016 | 0.016 | 0.014 | 14.8 | 5 |
| 8876 | VNN1 | cell membrane | | immunity, apoptosis, | 0.151 | 0.016 | 0.014 | 11.0 | 5 |
| | | | | cell adhesion | | | | | |
| 7490 | WT1 | nucleus | transcriptional | cell cycle | 0.164 | 0.019 | 0.014 | 100.6 | 5 |
| | | | regulation molecule | | | | | | |
| 11130 | ZWINT | nucleus | | cell cycle | 0.078 | 0.032 | 0.027 | 2.2 | 4 |

TABLE 3
List of primers, probes and PCR products used in qRT-PCR

| Gene | Primer/Probe | Sequence | Tm [° C.] | b.p. |
|---|---|---|---|---|
| Hs_ACTR2 | Hs_ACTR2-Probe | TCCTGGCGTGCCATGACGGTTGGA (SEQ ID NO: 1) | 64.7 | 24 |
| | Hs_ACTR2-F | GTGCTTTCTGGAGGGTCTAGTATG (SEQ ID NO: 2) | 63.9 | 24 |
| | Hs_ACTR2-R | CGTGGGTGTTCAATGCGGATC (SEQ ID NO: 3) | 69.9 | 21 |
| Hs_ADFP | Hs_ADFP-Probe | AGTGATGAGTGGGAGTGTGGTGAGGA (SEQ ID NO: 4) | 73.9 | 26 |
| | Hs_ADFP-F | GTAGAGTGGAAAAGGAGGATTGG (SEQ ID NO: 5) | 64.7 | 23 |
| | Hs_ADFP-R | TAGACCTTGGATGTTGGACAGG (SEQ ID NO: 6) | 65.8 | 22 |
| Hs_AK5 | Hs_AK5-Probe | CCTGATGGTGATGGCGGTGGGGATGA (SEQ ID NO: 7) | 65.9 | 26 |
| | Hs_AK5-F | GCTGCTCCATTGGTTAAATACTTCC (SEQ ID NO: 8) | 66.4 | 25 |
| | Hs_AK5-R | GTTGTCAAGTGCCATGCTGATG (SEQ ID NO: 9) | 67.7 | 22 |
| [symbol] s_ALOX5A | Hs_ALOX5AP-Probe | AGAACGCAGAGGACCCCTGGGTAGAT (SEQ ID NO: 10) | 76.4 | 26 |
| | Hs_ALOX5AP-F | AGTAGTTTGTCGGTTACCTAGGAG (SEQ ID NO: 11) | 80.5 | 24 |
| | Hs_ALOX5AP-R | GTAATAGTTGAATATGCCAGCAACG (SEQ ID NO: 12) | 64.1 | 26 |
| s_ARHGAP | Hs_ARHGAP18-Probe | TGAGGGTGTCGAGAATCTTGGAACCAAG (SEQ ID NO: 13) | 74.7 | 28 |
| | Hs_ARHGAP18-F | GGTCAGTGTGGAGTATGTCAAAG (SEQ ID NO: 14) | 61.8 | 23 |
| | Hs_ARHGAP18-R | CTTCAGTGTGTCCCTGTTTGC (SEQ ID NO: 15) | 64.0 | 21 |
| Hs_BIK | Hs_BIK-Probe | CGCCTGGCCCAGCTCTCCGAGG (SEQ ID NO: 16) | 68.9 | 22 |
| | Hs_BIK-F | AGATGGAGGTGAGCCTCAGG (SEQ ID NO: 17) | 66.7 | 20 |
| | Hs_BIK-R | TCAGTCTGGTCGTAGATGAAAGC (SEQ ID NO: 18) | 64.4 | 23 |
| Hs_CACN84 | Hs_CACN84-Probe | AGCGAATGAGGCACAGCAACGAGTCG (SEQ ID NO: 19) | 77.0 | 26 |
| | Hs_CACN84-F | CCAGAGGAATTTGTGGGTTACAG (SEQ ID NO: 20) | 66.4 | 23 |
| | Hs_CACN84-R | GACAAGCGGTTCCTAGTCTTCC (SEQ ID NO: 21) | 65.0 | 22 |
| Hs_CCL5 | Hs_CCL5-Probe | AACCCAGCAGTCGTCTTTGTCACCCG (SEQ ID NO: 22) | 76.7 | 26 |
| | Hs_CCL5-F | TGAAGGAGTATTTGTACAGGAGTGG (SEQ ID NO: 23) | 64.0 | 25 |
| | Hs_CCL5-R | TCGCGAACCCATTTGTTGTGTG (SEQ ID NO: 24) | 68.2 | 22 |
| Hs_CD33 | Hs_CD33-Probe | TACCACAGGGTGAGGGTCCCGGAAAC (SEQ ID NO: 25) | 77.1 | 26 |
| | Hs_CD33-F | CAGCAGTGGGCAGGAATGAC (SEQ ID NO: 26) | 68.1 | 20 |
| | Hs_CD33-R | TGGTCATCCATCTCCACAGTAGG (SEQ ID NO: 27) | 66.3 | 23 |
| Hs_CD3D | Hs_CD3D-Probe | TGTTCCCACCGTTCCCTCTACCCATG (SEQ ID NO: 28) | 76.2 | 26 |
| | Hs_CD3D-F | TGGTACTGGCTAGGGTTCTGTC (SEQ ID NO: 29) | 63.3 | 22 |
| | Hs_CD3D-R | TCCAGTCTTGTAATGTCTGACAGG (SEQ ID NO: 30) | 63.5 | 24 |
| Hs_CD93 | Hs_CD93-Probe | AGGGCCACCTCACTTTCAGCAGTCTG (SEQ ID NO: 31) | 74.7 | 26 |
| | Hs_CD93-F | AATGCGGGAGAGAGTTAGTCC (SEQ ID NO: 32) | 85.2 | 21 |
| | Hs_CD93-R | GTGGCTGGTGACTCTAGTGTC (SEQ ID NO: 33) | 61.4 | 21 |
| Hs_CD97 | Hs_Re_CD97-Probe | CGCGTTGGTGTACCTGCTGGAGTGG (SEQ ID NO: 34) | 76.0 | 25 |
| | Hs_Re_CD97-F | CTATGTGTTTACCATCCTCAACTGC (SEQ ID NO: 35) | 64.4 | 25 |
| | Hs_Re_CD97-R | GCGGACTTCGGGTATTCTTGC (SEQ ID NO: 36) | 67.5 | 21 |
| Hs_CLEC12 | Hs_CLEC12A-Probe | CCTGTCCACCAGAGTGCAAAGAATAGGCAG (SEQ ID NO: 37) | 76.7 | 30 |
| | Hs_CLEC12A-F | ACATGAATATGTCCAAGAAGATGAGG (SEQ ID NO: 38) | 64.9 | 26 |
| | Hs_CLEC12A-R | GCTGTCGTTATGCCAAATGCATC (SEQ ID NO: 39) | 67.3 | 23 |
| Hs_CTSC | Hs_CTSC-Probe | CCAGCGCGATGTCAAGTGCTGGGTT (SEQ ID NO: 40) | 78.7 | 26 |
| | Hs_CTSC-F | TCTTCCAGGTGGGGTCCAG (SEQ ID NO: 41) | 67.8 | 19 |
| | Hs_CTSC-R | GCCAGAATTGCCAAGGTCATG (SEQ ID NO: 42) | 67.7 | 21 |
| Hs_CYBB | Hs_CYBB-Probe | TGGCAAGAGGGTGAGAGCCAGGTACA (SEQ ID NO: 43) | 77.4 | 26 |
| | Hs_CYBB-F | TGATCCTTATTCAGTAGCACTCTCTG (SEQ ID NO: 44) | 63.5 | 26 |
| | Hs_CYBB-R | AGCGTGACAACTCCAGTC (SEQ ID NO: 45) | 65.3 | 21 |
| Hs_DDK2 | Hs_DDK2-Probe | GGTGTCCAGAGAGGGAGGGAGGGC (SEQ ID NO: 46) | 79.3 | 24 |
| | Hs_DDK2-F | AGCTGTACGACTGGCCCTAC (SEQ ID NO: 47) | 63.2 | 20 |
| | Hs_DDK2-R | TGCCGGGTTTCGAAGTCAAAG (SEQ ID NO: 48) | 70.1 | 21 |
| Hs_FCER1G | Hs_FCER1G-Probe | AGCACCAGGAACCAGGAGACTTACGA (SEQ ID NO: 49) | 72.2 | 26 |
| | Hs_FCER1G-F | GAGAAATCAGATGGTGTTTACACG (SEQ ID NO: 50) | 63.4 | 24 |
| | Hs_FCER1G-R | CTACTGTGGTGGTTTGTGATGG (SEQ ID NO: 51) | 63.4 | 22 |
| Hs_FCGR2A | Hs_FCGR2A-Probe | TGTCCCAGAAAGGTGTGGCTGCTTCA (SEQ ID NO: 52) | 76.7 | 26 |
| | Hs_FCGR2A-F | GATGACTATGGAGAGGCAAATGTC (SEQ ID NO: 53) | 64.4 | 24 |
| | Hs_FCGR2A-R | CAAGTTTCAGGACAGGGTTTGG (SEQ ID NO: 54) | 67.8 | 22 |
| Hs_FUCA2 | Hs_FUCA2-Probe | CCCAGTAGTTTCACCTCTGTTGGCGC (SEQ ID NO: 55) | 73.5 | 26 |
| | Hs_FUCA2-F | TTTGTTAAATGGCCCACATCAGG (SEQ ID NO: 56) | 67.5 | 23 |
| | Hs_FUCA2-R | GAAATCCAGTTAAGTGGGTGTCC (SEQ ID NO: 57) | 64.6 | 23 |
| Hs_FYB | Hs_FYB-Probe | AGCGAACCAGCATGAAAGCATCTCAC (SEQ ID NO: 58) | 78.1 | 26 |
| | Hs_FYB-F | ACCAGGTCCACCATCCCATC (SEQ ID NO: 59) | 68.3 | 20 |
| | Hs_FYB-R | ACAGCATGTTGATTGTCTTCATTGAC (SEQ ID NO: 60) | 66.9 | 28 |
| Hs_GPR34 | Hs_GPR34-Probe | TGGCCTTACTCCTCCCACAGAATGCG (SEQ ID NO: 61) | 76.4 | 26 |
| | Hs_GPR34-F | GATACCATAACAATGACGACAAGTTG (SEQ ID NO: 62) | 64.1 | 26 |
| | Hs_GPR34-R | CATGGGACAGGTAGTAACATTTGG (SEQ ID NO: 63) | 65.0 | 24 |
| Hs_GPR84 | Hs_GPR84-Probe | AGGGGAGCACAGACTGATGGTAGCAG (SEQ ID NO: 64) | 73.8 | 26 |
| | Hs_GPR84-F | CTTTGGGTGAGTTGAAGTTGTTCC (SEQ ID NO: 65) | 65.8 | 24 |
| | Hs_GPR84-R | CCCAGCTAACTGCAACATAACG (SEQ ID NO: 66) | 66.3 | 22 |
| Hs_HCK | Hs_HCK-Probe | ACCCTCGCTTCAGCCAGAGTTTCCTC (SEQ ID NO: 67) | 75.2 | 26 |
| | Hs_HCK-F | CCCTTCCTACTCCCAGACACC (SEQ ID NO: 68) | 65.8 | 21 |
| | Hs_HCK-R | AGAGATTTTCCAGTCGAACCTACC (SEQ ID NO: 69) | 64.3 | 24 |
| Hs_HCST | Hs_HCST-Probe | CCTGCTTTTGCTCCCAGTGGCTGC (SEQ ID NO: 70) | 76.9 | 24 |
| | Hs_HCST-F | GATCCATCTGGGTCACATCCTC (SEQ ID NO: 71) | 86.8 | 22 |
| | Hs_HCST-R | CAGGGTAAAAGGCAGGGAGTG (SEQ ID NO: 72) | 86.7 | 21 |
| Hs_HGF | Hs_HGF-Probe | TGTTGGGTTGTAGGTGGGTGGTTTACCA (SEQ ID NO: 73) | 74.4 | 28 |
| | Hs_HGF-F | GCCATGAATTTGACCTCTATGAAAAC (SEQ ID NO: 74) | 66.2 | 26 |
| | Hs_HGF-R | GCTGACATTTGATGCCAGTGTTAG (SEQ ID NO: 75) | 65.7 | 24 |
| Hs_HLX | Hs_HLX-Probe | CCTTCGTGAGCAGAGCATAGGGACCT (SEQ ID NO: 76) | 74.3 | 26 |
| | Hs_HLX-F | CAGTTCAGCATCAGTTCCAAGAG (SEQ ID NO: 77) | 64.9 | 23 |
| | Hs_HLX-R | TTGTACGTCGTCTGCGGCATGG (SEQ ID NO: 78) | 68.2 | 19 |
| Hs_HDMER3 | Hs_HDMER3-Probe | TCGCCCACGGAGCCCTCAGACAA (SEQ ID NO: 79) | 79.3 | 23 |
| | Hs_HDMER3-F | AAACTGTTCCGCAGCCAGAG (SEQ ID NO: 80) | 66.6 | 20 |
| | Hs_HDMER3-R | AACTCGGCCTCGCACTGTAC (SEQ ID NO: 81) | 65.3 | 20 |
| Hs_IL2RG | Hs_IL2RG-Probe | TCAGCCAGTCCCTTAGACACACCACT (SEQ ID NO: 82) | 71.5 | 26 |
| | Hs_IL2RG-F | GCTAGAGGATGTTGTTACTGAATACG (SEQ ID NO: 83) | 61.1 | 26 |
| | Hs_IL2RG-R | AGAGTCGTTGACTGTAGTCTGG (SEQ ID NO: 84) | 60.0 | 22 |
| Hs_IL3RA | Hs_IL3RA-Probe | TTCCGCGCGTCCCAGACCAGGA (SEQ ID NO: 85) | 65.1 | 22 |
| | Hs_IL3RA-F | CCCGCATCCCTCAGATGAAAG (SEQ ID NO: 86) | 70.7 | 21 |
| | Hs_IL3RA-R | AGTCACCAGACACTCCTCCAG (SEQ ID NO: 87) | 63.4 | 21 |
| Hs_ITGB2 | Hs_ITGB2-Probe | TCTGATCCACCTGAGCGACCTCCGG (SEQ ID NO: 88) | 78.4 | 26 |
| | Hs_ITGB2-F | TCCTGCTGGTCATCTGGAAGG (SEQ ID NO: 89) | 68.7 | 21 |
| | Hs_ITGB2-R | CAGCAAACTTGGGGTTGATGAG (SEQ ID NO: 90) | 67.5 | 22 |
| Hs_LGALS1 | Hs_LGALS1-Probe | TTCGTATCCATCTGGCAGCTTGACGGTCA (SEQ ID NO: 91) | 78.5 | 29 |
| | Hs_LGALS1-F | GCGGGAGGCTGTCTTTCC (SEQ ID NO: 92) | 67.2 | 18 |
| | Hs_LGALS1-R | CAGGTTGAGGCGGTTGGG (SEQ ID NO: 93) | 69.0 | 18 |
| Hs_LPXN | Hs_LPXN-Probe | TGCCAGCATCTGCTCTCACTGCAACC (SEQ ID NO: 94) | 77.6 | 26 |
| | Hs_LPXN-F | TGAAGCCCAAGAGCCAAAGG (SEQ ID NO: 95) | 68.5 | 20 |
| | Hs_LPXN-R | TCCTGCTTGTCTGGTAAGTGC (SEQ ID NO: 96) | 64.2 | 21 |
| Hs_LRG1 | Hs_LRG1-Probe | AGACCTTGCCACCTGACCTCCTGAG (SEQ ID NO: 97) | 73.8 | 26 |
| | Hs_LRG1-F | CCTTGACCTTGGGGAGAACC (SEQ ID NO: 98) | 66.8 | 20 |
| | Hs_LRG1-R | TTCTAGATGTAGCCGTTCTAATTGC (SEQ ID NO: 99) | 63.2 | 25 |
| Hs_LY86 | Hs_LY86-Probe | CTATCCCATCTGTGTGAGGCGGCTCTGC (SEQ ID NO: 100) | 76.4 | 26 |
| | Hs_LY86-F | ATGTCTCAAGGCTCATGTTTG (SEQ ID NO: 101) | 65.3 | 24 |
| | Hs_LY86-R | TGAGAGGCCCAGCATAGTAAATC (SEQ ID NO: 102) | 65.9 | 23 |
| Hs_MGAT4A | Hs_MGAT4A-Probe | TTGGCTCCTGGACCATATTCTCTGGGT (SEQ ID NO: 103) | 74.1 | 27 |
| | Hs_MGAT4A-F | ATATTCATGTTTTACAAGGAGAAACCC (SEQ ID NO: 104) | 63.8 | 27 |
| | Hs_MGAT4A-R | AGATTTGCTTTGTGTCTATCACAATG (SEQ ID NO: 105) | 63.2 | 26 |
| Hs_NCF4 | Hs_NCF4-Probe | GGTTGACCGCATGGCAGCTCCGAG (SEQ ID NO: 106) | 66.4 | 24 |
| | Hs_NCF4-F | AGAGCGTGTCCCCACAGG (SEQ ID NO: 107) | 66.5 | 18 |
| | Hs_NCF4-R | CGACTGAGGAGGAAGATCACATC (SEQ ID NO: 108) | 66.1 | 23 |
| Hs_NEK6 | Hs_NEK6-Probe | ACTGGTCAGCATGTGCATCTGCCCTG (SEQ ID NO: 109) | 77.4 | 26 |
| | Hs_NEK6-F | GGGGAGCACTACTCCGAGAAG (SEQ ID NO: 110) | 66.2 | 21 |
| | Hs_NEK6-R | CACGTATCCGATGTCAGGTCTC (SEQ ID NO: 111) | 25.6 | 22 |
| Hs_P2RY5 | Hs_P2RY5-Probe | ACGCTTACCATCGTAAAGGCAGGTCCAATT (SEQ ID NO: 112) | 75.5 | 30 |
| | Hs_P2RY5-F | AGAGGTTATAATCTGGAATCCCAAAGG (SEQ ID NO: 113) | 64.1 | 26 |
| | Hs_P2RY5-R | TAAAGGAGTCATTATAGAAGCAGTGG (SEQ ID NO: 114) | 62.4 | 25 |
| Hs_PDE9A | Hs_PDE9A-Probe | TCCACCCAAGGCTCTGCGACTTCCAT (SEQ ID NO: 115) | 78.0 | 26 |
| | Hs_PDE9A-F | GAGTACAGCAAGGAGGAGCAC (SEQ ID NO: 116) | 63.9 | 21 |
| | Hs_PDE9A-R | GGTCGCTCGCATAAAATATTCCTC (SEQ ID NO: 117) | 66.4 | 25 |
| Hs_PDK1 | Hs_PDK1-Probe | TCGTGTTGAGACCTCCCGCGCAGT (SEQ ID NO: 118) | 78.2 | 24 |
| | Hs_PDK1-F | ACATGTATTCAACTGCACCAAGAC (SEQ ID NO: 119) | 63.8 | 24 |
| | Hs_PDK1-R | TGGGCAATCCATAACCAAAACC (SEQ ID NO: 120) | 68.3 | 22 |
| Hs_PRKCD | Hs_PRKCD-Probe | TTGCCGTAGGTCCCACTGTTGTCTTGC (SEQ ID NO: 121) | 76.3 | 27 |
| | Hs_PRKCD-F | GATGAGACTCAGCCTCCTCAGA (SEQ ID NO: 122) | 64.9 | 22 |
| | Hs_PRKCD-R | GCTGGTGCCCTCCCAGAT (SEQ ID NO: 123) | 68.0 | 18 |
| Hs_PRSS21 | Hs_PRSS21-Probe | AGACCCCTCCTGGCCGCTACTCTTTT (SEQ ID NO: 124) | 74.2 | 26 |
| | Hs_PRSS21-F | TGGCCCAGAGTGGCATGTC (SEQ ID NO: 125) | 69.9 | 19 |
| | Hs_PRSS21-R | GGCTCAGGTAGGCTCAGACC (SEQ ID NO: 126) | 85.2 | 20 |
| Hs_PTH2R | Hs_PTH2R-Probe | CCACCACCTCGCATCAGCCAGAGTTG (SEQ ID NO: 127) | 76.5 | 26 |
| | Hs_PTH2R-F | GGGTTTCCAGCAGCATTTGTTG (SEQ ID NO: 128) | 66.1 | 22 |
| | Hs_PTH2R-R | CCACTTGATGTCTCCAGCACTAAG (SEQ ID NO: 129) | 68.1 | 24 |
| Hs_RAB20 | Hs_RAB20-Probe | ACATCTCCATCTGGGACACCGCAGGG (SEQ ID NO: 130) | 78.8 | 26 |
| | Hs_RAB20-F | GCCTTCTACCTGAAGCAGTGG (SEQ ID NO: 131) | 66.0 | 21 |
| | Hs_RAB20-R | TGCCGGTGATTCACATCATAGG (SEQ ID NO: 132) | 68.8 | 22 |
| Hs_RAB8A | RAB8A-Probe | TTTTGACTCCCTGGTTGCTCCCCTGG (SEQ ID NO: 133) | 77.1 | 26 |
| | Hs_RAB8A-F | GCCAACATCAATGTGGAAAATGC (SEQ ID NO: 134) | 69.0 | 23 |
| | Hs_RAB8A-R | GTGCTCCTCTTCTGCTGGTC (SEQ ID NO: 135) | 64.3 | 20 |
| Hs_RABIF | Hs_RABIF-Probe | CGCCGTCAGGATTCCTGCCGTCA (SEQ ID NO: 136) | 64.9 | 23 |
| | Hs_RABIF-F | AGGGACCGCTCTCTTCTCTG (SEQ ID NO: 137) | 63.9 | 20 |
| | Hs_RABIF-R | CCTCAACCAGCCACTCTTCC (SEQ ID NO: 138) | 66.9 | 20 |
| Hs_RNSE2 | Hs_RNSE2-Probe | AGTCTCCGCGCTGTAGCTCCTGTGA (SEQ ID NO: 139) | 74.7 | 25 |
| | Hs_RNSE2-F | CCCTGAACCCCAGAAGAACC (SEQ ID NO: 140) | 87.7 | 20 |
| | Hs_RNSE2-R | GGGAAGTGAACAGTTTTGGAACC (SEQ ID NO: 141) | 66.5 | 23 |
| Hs_SLC43A | Hs_SLC43A-Probe | CCTTGTCGGCTGTGGTGTCTCTGCTC (SEQ ID NO: 142) | 76.3 | 26 |
| | Hs_SLC43A-F | AAGCTCTTTGGGCTGGTGATG (SEQ ID NO: 143) | 67.5 | 21 |
| | Hs_SLC43A-R | GGTGGAAGAATGTCAGAAGAATGG (SEQ ID NO: 144) | 66.6 | 24 |
| Hs_SUCNR1 | Hs_SUCNR1-Probe | AAGGACTCCCACAACGAACTCAATCCCA (SEQ ID NO: 145) | 76.6 | 28 |
| | Hs_SUCNR1-F | TACGACATGCTGGGGATCATG (SEQ ID NO: 146) | 68.0 | 21 |
| | Hs_SUCNR1-R | GTAGCCGTAAACAACAATGGTATTTC (SEQ ID NO: 147) | 64.1 | 26 |
| Hs_TIMP1 | Hs_TIMP1-Probe | TGGTCCGTCCACAAGCAATGAGTGCC (SEQ ID NO: 148) | 78.6 | 26 |
| | Hs_TIMP1-F | ACTGTTGGCTGTGAGGAATGC (SEQ ID NO: 149) | 66.4 | 21 |
| | Hs_TIMP1-R | CCTTTTCAGAGCCTTGGAGGAG (SEQ ID NO: 150) | 87.2 | 22 |
| Hs_TNF | Hs_TNF-Probe | CCCGAGTGACAAGCCTGTAGCCCAT (SEQ ID NO: 151) | 75.1 | 26 |
| | Hs_TNF-F | CCAGGCAGTCAGATCATCTTCTC (SEQ ID NO: 152) | 66.2 | 23 |
| | Hs_TNF-R | GTGTCAGCTCCAGGCCATTG (SEQ ID NO: 153) | 66.3 | 20 |
| Hs_TNFRSF [symbol] | Hs_TNFRSF4-Probe | CCTTGGCTGGGAAGCACACCCTGC (SEQ ID NO: 154) | 78.5 | 24 |
| | Hs_TNFRSF4-F | CCTGCAAGCCCTGGACCA (SEQ ID NO: 155) | 70.0 | 18 |
| | Hs_TNFRSF-R | AGATTGCGTCCGAGCTATTGC (SEQ ID NO: 156) | 67.4 | 21 |
| [symbol] s_TNFSF13 [symbol] | Hs_TNFSF13B-Probe | TCTTCTGGACCCTGAACGGCACGCT (SEQ ID NO: 157) | 77.8 | 25 |
| | Hs_TNFSF13B-F | ACCAGCTCCAGGAGAAGGC (SEQ ID NO: 158) | 65.8 | 19 |
| | Hs_TNFSF13B-R | CAGTTGCAAGCAGTCTTGAGTG (SEQ ID NO: 159) | 65.0 | 22 |
| Hs_TXNL4B | Hs_TXNL4B-Probe | CTCCTTACTCGTCCACGCCGCCTCA (SEQ ID NO: 160) | 77.8 | 26 |
| | Hs_TXNL4B-F | TCCGAGAAGTGGTTGCTGAC (SEQ ID NO: 161) | 65.4 | 20 |
| | Hs_TXNL4B-R | TCTTGAAATAACCCAAATGTGAATCC (SEQ ID NO: 162) | 66.3 | 26 |
| Hs_TYR08P | Hs_TYR08P-Probe | CGCTGTAGACATCCGACCTCTGACCC (SEQ ID NO: 163) | 74.9 | 26 |
| | Hs_TYR08P-F | CTGAGACCGAGTCGCCTTATC (SEQ ID NO: 164) | 65.0 | 21 |
| | Hs_TYR08P-R | ATACGGCCTCTGTGTGTTGAG (SEQ ID NO: 165) | 64.0 | 21 |
| Hs_VNN1 | Hs_VNN1-Probe | AGTACCGATAACAGCCATGCACTGTGC (SEQ ID NO: 166) | 72.9 | 27 |
| | Hs_VNN1-F | AGTGCTGTGATGATGGACAATTAC (SEQ ID NO: 167) | 63.8 | 24 |
| | Hs_VNN1-R | TTGCTCTCCTGTGCAGAAGG (SEQ ID NO: 168) | 65.8 | 20 |
| Hs_MT1 | Hs_MT1-Probe | TCTCACCAGTGTGCTTCCTGCTGTGC (SEQ ID NO: 169) | 76.1 | 26 |
| | Hs_MT1-F | GTCGGCATCTGAGACCAGTG (SEQ ID NO: 170) | 66.0 | 20 |
| | Hs_MT1-R | GTTCACAGTCCTTGAAGTCACAC (SEQ ID NO: 171) | 62.6 | 23 |
| Hs_ZWINT | Hs_ZWINT-Probe | CCACTGGTTCTGGACTGCTCTGCGTT (SEQ ID NO: 172) | 76.5 | 26 |
| | Hs_ZWINT-F | CCCAGAGGAAACGGACACAAC (SEQ ID NO: 173) | 67.8 | 21 |
| | Hs_ZWINT-R | TGGAGATGCTTCTCCTGTTGTAG (SEQ ID NO: 174) | 65.4 | 23 |

| | | PCR |
|---|---|---|
| | | product |
| Gene | Prospective sequence of PCR product | b.p. |
| Hs_ACTR2 | GTGCTTTCTGGAGGGTGTATCCTGGCCTCGGATCACGGTTGGAACGAGAAC | 140 |
| | TTAAACAGCTTTAGTTAGAACGAGTTTTGAAGGGTGATGTGGAAAAACTTTCTAAATT | |
| | TAAGATCCGCATTGAAGACCCACC (SEQ ID NO: 175) | |
| Hs_ADFP | GTAGAGTGGAAAAGGAGGATTGGATATGATGATACTGATGAGTCCCACTGTCCTGAGC | 150 |
| | TCATTGAGTGACCTACTCTTGCAATTGCCCGGAACCTGACTGAGCAGCTGCAGACCAG | |
| | GTGCCACACCCTCCTGTCCAACATCCAAGGTGTA (SEQ ID NO: 176) | |
| Hs_AK5 | GCTGGTCCATTGGTTAAATAGTTCCAGGAAAAGGGGCTCATCATGACATTTGATGGGG | 108 |
| | ACCGCGATGAGGATGAGGTGTTCTATGACATCAGCATGGCAGTTGACAAC (SEQ ID | |
| | NO: 177) | |
| [symbol] s_ALOX5A | AGTACTTTGTCGGTTACGTAGGAGAGAGAACGCAGAGGACCCCTGGCTACATATTTGG | 116 |
| | GAAACGGATCATACTCTTCCTGTTCGTGATGTCCGTTGGTGGCATATTGAAGTATTAC | |
| | (SEQ ID NO: 178) | |
| s_ARHGAP | GGTGAGTGTGGAGTATCTCAAAGGCTTTCAGGGTGTCCAGAATCTTGGAACCAAGAAG | 124 |
| | CAGCAACTACAGGCTTTGAACCTTCTTGTCATCCTCCTACCTGATGCAAACAGGGACA | |
| | GAGTGAAG (SEQ ID NO: 179) | |
| Hs_BIK | TCGGGGAGCAGATGGACGTGAGCCTCAGGGCCCCGGGCCTGGCCCAGCTCTCCGAGGT | 103 |
| | GGCGATGGACAGCCTGGGTGTGGCTTTCATGTAGGACCAGACTGA (SEQ ID NO: | |
| | 180) | |
| Hs_CACN84 | GCAGAGGAATTTCTGGGTTAGAGAGTCAGCGAATGAGGCACAGCAACCAGTGGAGAGA | 145 |
| | GAAGTGTGGAATTGAAAGAGGAAGTGTAATGACCTCTGATGAAAATTATCAGAATGAA | |
| | AGGGGTGGGAAGAGTAGGAACCGGTTGTC (SEQ ID NO: 181) | |
| Hs_CCL5 | TCAAGGAGTATTTCTACACCAGTGGCAAGTGCTCCAACCCAGCAGTCGTCTTTGTCAC | 109 |
| | CGGAAAGAACGGCCAAGTGTGTGCCAACCCAGAGAAGAAATGGGTTCGGGA (SEQ | |
| | ID NO: 182) | |
| Hs_CD33 | GAGGAGTGGGGAGGAATGAGACCCAGGGTAGGACAGGGTCAGGGTCCCCGAAACACCA | 136 |
| | GAAGAAGTCCAAGTTACATGGGCGCACTGAAACCTGAAGGTGTTCAGGTGCGGCCGGT | |
| | ACTGTGGAGATGGATGAGGA (SEQ ID NO: 183) | |
| Hs_CD3D | TGGTACTGGCTACCCTTCTCTCGCAAGTGAGCCCCTTCAAGATACCTATAGAGGAACT | 148 |
| | TGAGGACAGAGTGTTTGTGAATTGCAATACCAGGATCAGATGGGTAGAGGGAACGGTG | |
| | GGAACACTGCTCAGACATTACAAGACTGGA (SEQ ID NO: 184) | |
| Hs_CD93 | AATGCGGCAGAGAGTTACTCCTGGGTTCCAGAGCGAGGTGAGAGCAGGGGGATGGAGA | 132 |
| | AGCAGTACAGTGCGACAGGTGGGACAGAGTGGTGAAAGTGAGGTGGGCCTAGAGACAC | |
| | TAGAGTCACCAGCCAC (SEQ ID NO: 185) | |
| Hs_CD97 | CTATGTGTTTACCATCGTCAACTGGGTGGAGGGCGCCTTCCTCACCTGCTGCACTGC | 99 |
| | CTGCTCAACAAGAAGGTTCGGGAAGAATACCGGAAGTGGGC (SEQ ID NO: | |
| | 186) | |
| Hs_CLEC12 | ACATGAATATGTCCAACAAGATCAGGAACCTGTGGACCACAGTGCAAACAATAGCCAC | 143 |
| | GAAATTATGTGGTGAGGTATATAGGAAAGAAGAAGAGGACAAATGTAAGCGTTGTCCA | |
| | AGGAGATGGATTTGGCATAAGGAGAGC (SEQ ID NO: 187) | |
| Hs_CTSC | TCTTCCAGGTGGGCTCCAGCGGTTGGCAGCGCGATGTCAACTGCTCGGTTATGGGACC | 128 |
| | ACAAGAAAAAAAAGTAGTGGTGTACCTTCAGAAGGTGGATACAGCATATGATGAGGTT | |
| | GGGAATTGTGGG (SEQ ID NO: 188) | |
| Hs_CYBB | TGATCCTTATTCAGTAGCACTCTCTGAACTTGGAGACAGGCAAAATGAAAGTTATCTC | 150 |
| | AATTTTGGTGGAAAGAGAATAAAGAACCCTGAAGGAGGCCTGTACCTGGCTGTGACCC | |
| | TGTTGGCAGGCATCACTGGAGTTGTGATGACGCT (SEQ ID NO: 189) | |
| Hs_DDK2 | AGCTGTACGACTGGCCCTACAGGTTTCTGCGGCGCTTTGGGCGGGACAAGGTAACCTT | 121 |
| | TTCCTTTGAGGCAGGCCGTCGCTGCGTCTCTGGAGAGGGCAACTTTGAGTTGGAAACG | |
| | CGGCA (SEQ ID NO: 190) | |
| Hs_FCER1G | GAGAAATCAGATGGTGTTTACACGGGCCTGAGCACCAGGAACCAGGAGACTTACGAGA | 87 |
| | CTGTGAAGCATGAGAAACCACCACAGTAG (SEQ ID NO: 191) | |
| Hs_FCGR2A | GATGACTATGGAGACGCAAATGTCTCAGAATGTATGTCCCAGAAACCTGTGGCTGCTT | 137 |
| | GAACCATTGACAGTTTTGGTGCTGCTGGGTTGTGCAGACAGTGAAGCTGCAGCTCCCC | |
| | CAAAGGCTGTGCTGAAACTTG (SEQ ID NO: 192) | |
| Hs_FUCA2 | TTTGTTAAATGGCCCACATCAGGACAGGTGTTGGTTGGGGATCCGAAAGGTATTCTGG | 110 |
| | GGGCAACAGAGGTGAAACTACTGGGCCATGGAGAGCCACTTAACTGGATTTC (SEQ | |
| | ID NO: 193) | |
| Hs_FYB | ACCACCTCCACCATCCCATCCGGCCAGCGAACCACCATTGCCAGCATGTGAGGGATGA | 147 |
| | CAACGACCAGTCCCAAGCCTACCTGGCAGAAACATTAAAGCTCCGTTTGAGGTAAAAA | |
| | GCGGTGTGAATGAAGACAATCAAGATGGTGT (SEQ ID NO: 194) | |
| Hs_GPR34 | CATACCATAACAATGACGACAAGTTCAGTGAGCAGCTGGGGTTACTCCTCCCACAGAA | 135 |
| | TGCGCTTTATAACCAATCATAGCGACCAACCGCCACAAAACTTCTCAGCAACACCAAA | |
| | TGTTACTACCTGTCCCATG (SEQ ID NO: 195) | |
| Hs_GPR84 | CTTTGGGTGAGTTGAACTTGTTCCATTATAGAAAGAATTGAAGGGTGAGAAACTCAGC | 144 |
| | CTCTATCATGTGGAACAGCTCTGACGCCAACTTCTCCTGGTACCATGAGTGTGTGCTG | |
| | CGCTATCGTTATGTTGCAGTTAGCTGGG (SEQ ID NO: 196) | |
| Hs_HCK | CCCTTCGTACTCCCAGACAGGGAGGGTCGGTTGAGGGACAGTTTCCTGATGTGTCCAG | 84 |
| | TGGGTAGGTTGGACTGGAAAATCTCT (SEQ ID NO: 197) | |
| Hs_HCST | GATCCATCTGGGTCACATCCTCTTCCTGCTTTTGCTCCCAGTGGCTGCAGCTCAGACG | 98 |
| | ACTCCAGGAGAGAGATCATCACTCCCTGCCTTTTACCCTG (SEQ ID NO: 198) | |
| Hs_HGF | GCCATGAATTTGACCTCTATGAAAACAAAGACTACATTAGAAACTGCATCATTGGTAA | 114 |
| | AGGACGCAGCTACAAGGGAACAGTATCTATCACTAAGAGTGGCATCAAATGTGAGC | |
| | (SEQ ID NO: 199) | |
| Hs_HLX | CAGTTCAGCATCAGTTCCAAGACAGGTTTCCAGGTCCCTATGCTGTGCTCACGAAGGA | 79 |
| | CACCATGCGGCAGACGTACAA (SEQ ID NO: 200) | |
| Hs_HDMER3 | AAAGTGTTCGGCAGCAGAGCGCTGATGCCCCCGGCCCCACAGAGCGCGAGCGGCTAA | 110 |
| | AGAAGATGTTGTCTGAGGGCTCCGTGGGCGAGGTAGAGTGGGAGGCCGAGTT (SEQ | |
| | ID NO: 201) | |
| Hs_IL2RG | CCTAGAGGATCTTGTTACTGAATACCACGGGAACTTTTCGGCCTGGAGTGGTGTGTGT | 304 |
| | AAGGGAGTGGCTGAGAGTGTGCAGGGAGAGTAGAGTGAACGACTGT (SEQ ID NO: | |
| | 202) | |
| Hs_IL3RA | CCGGCATCGGTCACATGAAAGACCCCATGGGTGACAGCTTCCAAAACGAGAAGGTGGT | 104 |
| | GGTGTGGGAGGCGGGCAAAGGGGGCCTGGAGGAGTGTCTGGTGACT (SEQ ID NO: | |
| | 203) | |
| Hs_ITGB2 | TCCTGCTGGTCATCTGGAAGGCTCTGATCCACCTGAGCGACCTCCGGGAGTACAGGCG | 150 |
| | CTTTGAGAAGGAGAAGCTCAAGTCCGAGTGGAACAATCATAATCCCCTTTTCAAGAGC | |
| | GCCACCAGGACGGTCATGAACCCCAAGTTTGCTG (SEQ ID NO: 204) | |
| Hs_LGALS1 | GCGGGAGGCTGTCTTTCCCTTCCAGCCTGGAAGTGTTGCAGAGGTGTGCATGACCTTG | 127 |
| | GACCAGGCCAACCTGACCGTCAAGCTGCCAGATGGTAGGAATTCAAGTTCCCCAACC | |
| | GCCTCAACCTG (SEQ ID NO: 206) | |
| Hs_LPXN | TGAAGCCCAAGAGCCAAAGGAATCACCACCACCTTCTAAAACGTCAGCAGCTGCTCAG | 150 |
| | TTGGATGAGCTCATGGCTCACCTGACTGAGATGCAGGCCAAGGTTGCAGTGAGAGCAG | |
| | ATGCTGGCAAGAAGCACTTACCAGACAACCAGGA (SEQ ID NO: 206) | |
| Hs_LRG1 | CCTTGACCTTGGGGAGAACCAGTTGGAGACCTTGCCACCTGACCTCGTGAGGGGTCCG | 85 |
| | CTGCAATTAGAACGGCTACATGTAGAA (SEQ ID NO: 207) | |
| Hs_LY86 | ATGTGTCAAGGCTCATCTGTTTTGAATTTGTCCTATCCCATCTGTGAGGCGGGTCTGC | 118 |
| | CCAAGTTTTCTTTCTGTGGAAGAAGGAAAGGAGAGCAGATTTACTATGCTGGGCCTGT | |
| | CA (SEQ ID NO: 208) | |
| Hs_MGAT4A | ATATTCATGTTTTACAAGGAGAAACCCATTGATTGGCTCCTGGACCATATTCTCTGGG | 116 |
| | TGAAAGTCTGCAACCCTGAAAAAGATGCAAAAGATTGTGATAGACACAAAGCAAATCT | |
| | (SEQ ID NO: 209) | |
| Hs_NCF4 | AGAGCGTGTCCCCACAGGGCAACAGCGTGACCGCATGGCAGCTCCGAGAGCAGAGGC | 133 |
| | TCTATTTGACTTCACTGGAAACAGCAAACTGGAGCTGAATTTCAAAGCTGGAGATGTG | |
| | ATCTTCCTCCTCAGTCG (SEQ ID NO: 210) | |
| Hs_NEK6 | GGGGAGCACTACTCCGAGAAGTTACGAGAACTGGTCAGCATGTGCATGTGCCCTGACC | 87 |
| | CCCACCAGAGACCTGACATCGGATACGTG (SEQ ID NO: 211) | |
| Hs_P2RY5 | AGAGGTTATAATCTGAATCCCAAAGGAGACTGCAGCTGATGAAAGTGCTTCCAAACTG | 125 |
| | AAAATTGGACGTGCCTTTACGATGGTAAGGGTTAACACCTCCCACTGGTTCTATAATG | |
| | ACTCCTTTA (SEQ ID NO: 212) | |
| Hs_PDE9A | GACTACAGCAAGGACCAGCACATGACCCTGCTGAAGATGATTTTGATAAAATGCTGTG | 142 |
| | ATATCTCTAACGAGGTCCGTCCAATGGAAGTCGCAGAGCCTTGGGTGGACTCTTTATT | |
| | AGAGGAATATTTTATCCAGAGCGACC (SEQ ID NO: 213) | |
| Hs_PDK1 | AGATGTATTCAACTGCACCAAGACCTCGTGTTGAAGACCTCCCGCGCAGTGCCTCTGGC | 81 |
| | TGGTTTTGGTTATGGATTGCCCA (SEQ ID NO: 214) | |
| Hs_PRKCD | GATCAGACTGAGCCTCCTCAGACCCTGTTGGGATATACAGGGTTTGGACAAGAAGAC | 125 |
| | CGGAGTTGCTGGGGAGGACATGCAACACAACAGTGGACCTACGGCAAGATCTGGGAG | |
| | GGCAGCAGG (SEQ ID NO: 215) | |
| Hs_PRSS21 | TGGCCCCAGAGTGGCATGTCCCAGCCAGACCCCTCCTGCCCGCTACTCTTTTCCCTCT | 105 |
| | TCTCTGGGCTCTCCCACTCCTGGGGCCGGTCTGAGCCTACCTGAGCC (SEQ ID | |
| | NO: 216) | |
| Hs_PTH2R | GGGTTTCCAGCAGCATTTGTTGCAGCATGGGCTGTGGGACGAGCAACTCTGGGTGATG | 95 |
| | CGAGGTGCTGGGAACTTAGTGCTGGAGAGATGAAGTGG (SEQ ID NO: 217) | |
| Hs_RAB20 | GCCTTCTACCTGAAGCAGTGGCGGCTGGTACAACATCTCCATCTGGGACACCGCAGGGC | 140 |
| | GGGAGCAGTTCCAACGGCCTGGGTCCATGTACTGCCGGGGGGCGGGGGGGATCATCCT | |
| | CACCTAGATGTGAATCACCGGCA (SEQ ID NO: 218) | |
| Hs_RAB8A | GCCAACATCAATGTGGAAAATGGATTTTTCAGTGTCGCCAGAGATATCAAAGCAAAA | 140 |
| | TGGACAAAAAATTGGAAGGCAACAGCCCCCAGGGGAGCAACCAGGGAGTCAAAATCAC | |
| | ACCGGACCAGCAGAAGAGGAGCAG (SEQ ID NO: 219) | |
| Hs_RABIF | AGGGACCCCTCTCTTCTCTCGCCCACAGCTTTTCCTTCCCTCCATGAGAAAGAAGCCA | 119 |
| | GCTCTGTCTGACGGGAGCAATCCTGAGGGCCATCTCCTGCAGGAACACTGGGTGGTTG | |
| | AGG (SEQ ID NO: 220) | |
| Hs_RNSE2 | CCCTGAACCCCAGAACAACCAGCTGGATCAGTTCTCACAGGAGCTACAGCGCGGAGAC | 91 |
| | TGGGAAACATGGTTCCAAAACTGTTCACTTCCC (SEQ ID NO: 221) | |
| Hs_SLC43A | AAGCTCTTTGGGCTGGTGATGGCCTTGTCGGCTCTGGTGTCTCTGCTCCAGTTCCCA | 148 |
| | TCTTCACCCTCATCAAAGGCTCCCTTCAGAATGACCCATTTTACGTGAATGTGAGTT | |
| | CATGCTTGCCATTCTTCTGACATTCTTCCACC (SEQ ID NO: 222) | |
| Hs_SUCNR1 | TACGACATGCTGGGGATCATGGCATGGAATGCAACTTGCAAAAACTGGCTGGCAGCAG | 150 |
| | AGGCTGCCCTGGAAAATACTACCTTTCCATTTTTTATGGGATTGAGTTCGTTGTGGG | |
| | AGTCCTTGGAAATACCATTGTTGTTTACGGCTAC (SEQ ID NO: 223) | |
| Hs_TIMP1 | ACTGTTGGCTGTGAGGAATGCACAGTGTTTCCCTGTTTATCCATCCCCTGCAAACTGC | 112 |
| | AGAGTGGCACTGATTGCTTGTCCACGGACCAGCTCCTCCAAGGCTCTGAAAAGG | |
| | (SEQ ID NO: 224) | |
| Hs_TNF | CCAGGCAGTCAGATCATCTTCTCGAACCCCGAGTGACAAGCCTGTAGCCCATGTTGTA | 140 |
| | GCAAACCCTCAAGCTGAGGGGCAGCTCCAGTGGCTGAACCGCCGGGCCAATGCCCTCG | |
| | TGGCCAATGGCGTGGAGCTGAGAG (SEQ ID NO: 225) | |
| Hs_TNFRSF [symbol] | CCTGCAAGCCCTGGACCAACTGCACCTTGGCTGGGAAGCACACCCTGCAGCCGGCCAG | 78 |
| | CAATAGCTCGGACGAATCT (SEQ ID NO: 226) | |
| [symbol] s_TNFSF13 [symbol] | ACCAGCTCCAGGAGAAGGCAACTCCAGTCAGAACAGCAGAAATAAGCGTGCCGTTCAG | 97 |
| | GGTCCAGAAGAAACAGTCACTCAAGACTGCTTGCAACTG (SEQ ID NO: 227) | |
| Hs_TXNL4B | TCCGAGAAGTGGTTGCTGACAGCCACAAAGTGAAAGGGAGTGAGGCGGCGTGGACGAG | 100 |
| | TAAGGAGTGACAGTGAGGATTCACATTTGGGTTATTTCAAGA (SEQ ID NO: | |
| | 228) | |
| Hs_TYR08P | CTGAGACCGAGTCGCCTTATCAGGAGCTCCAGGGTCAGAGGTCGGATGTCTACAGCGA | 80 |
| | CCTCAACACACAGAGGCCGTAT (SEQ ID NO: 229) | |
| Hs_VNN1 | AGTGCTGTGATGATGGACAATTACATAGTACCCATAACAGCCATGCACTGTCCAAAGC | 82 |
| | ATGCCCTTGTGCACAGGACAGCAA (SEQ ID NO: 230) | |
| Hs_MT1 | GTCGGCATCTGAGACCAGTGAGAAACGCCCCTTCATGTGTGCTTACCCAGGCTGCAAT | 149 |
| | AAGAGATATTTTAAGCTGTCCCACTTAGAGATGCACAGGAGGAAGCACACTGGTGAGA | |
| | AACCATACCAGTGTGACTTCAAGGACTGTGAAC (SEQ ID NO: 231) | |
| Hs_ZWINT | CCCAGAGGAAACGGACACAACTCCGGGAGCCTTTGAGCAGCTCCAGGCCAAGAACA | 124 |
| | AATGGCCATGGAGAAACGCAGAGCACTCCACAACCAGTGGCAGCTACAACAGGAGAAG | |
| | CATCTGCA (SEQ ID NO: 232) | |

[symbol]

indicates data missing or illegible when filed

For subsequent qPCR analysis, 121 genes that encode molecules in the following categories were selected from among the 217 genes identified in microarray experiments:

1) Those located on the cell membrane or in extracellular spaces,
2) cytokines, growth factors, transmembranous receptors, protein kinases, phosphatases, transcriptional regulation molecules, and/or signaling molecules, and
3) those involved in immune regulation, cell cycle, apoptosis, and/or cell adhesion.

The list includes 57 genes, the mRNA levels of which were significantly (P<0.05; according to Kruskal-Wallis, Wilcoxon-Mann-Whitney or Student's t-test) higher in LSCs than in HSCs. The columns in Table 2 indicates, in the order from the left column, Entrez Gene ID (Column A), HUGO Gene Symbol (Column B) localization (Column C), molecular function (Column D), biological process (Column E), P values from each statistical test (Columns F-H), ratio of median values of the mRNA levels (Column I), and the number of LSC samples showing a higher expression level than the mRNA levels for the HSC samples (Column J).

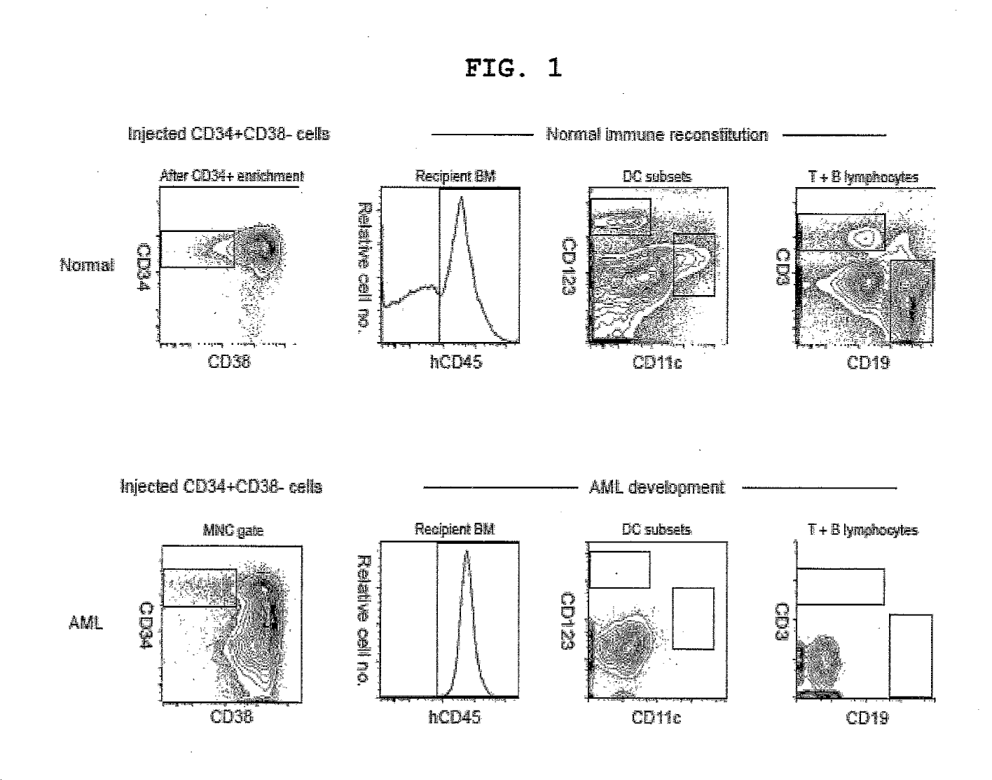

The present inventors previously reported that LSCs derived from bone marrow (BM) of AML patient origin and LSCs derived from BM of a mouse receiving transplantation of AML patient BM have similar transcription profiles (Nature Biotechnology, 2007, ibid). Based on this finding, the present inventors performed a comprehensive transcriptome analysis to compare LSCs and normal hematopoietic stem cells (HSCs), using two array platforms: Human Genome U133 plus 2.0 GeneChips (BM derived from 16 AML patients and BM derived from 5 AML transplantation recipient mice were compared with BM derived from 2 healthy donors and cord blood (CB) derived from 5 healthy donors) and Human Gene 1.0ST GeneChips (BM derived from 1 AML patient and BM derived from 5 AML transplantation recipient mice were compared with CB from 1 healthy donor and BM from 4). Since a previous study had revealed that AML stem cells are present exclusively in the CD34+CD38− fraction, >1.2×10⁴ CD34+CD38− cells were recovered with a purity of >98% (FIG. 1). Using the same method, CD34+CD38− HSCs were also purified from normal BM and CB samples (FIG. 1). By intravenously injecting the aforementioned purified HSCs and LSCs to neonatal NOD/SCID/IL2rg KO mice, the onset of AML by LSCs and the lack of reconstitution of normal immunity were confirmed, and long-time transplantation and multi-lineage (T/B/bone marrow) differentiation of HSCs were confirmed (FIG. 1). Not the CD34+CD38+ cells or CD34− cells derived from the AML transplantation recipient mice, but the CD34+CD38− bone marrow cells caused leukemia in secondary recipients. These data suggest that the transplanted CD34+CD38− cells did not come from the HSCs, but retained the nature of the LSCs. To analyze the expression data set obtained, genes that exhibit a significantly higher (p value <0.01, percentage of false positively <0.05) array signal in LSCs than in HSCs on both the two microarray platforms were extracted using RankProd (Bioinformatics 22, 2825, 2006) mounted on the Bioconductor package. A total of 217 gene candidates met the criteria (FIG. 5, Table 1); further, IL2R was added to make a total of 218 gene candidates.

Next, to demonstrate the expression levels of candidates for separating LSCs and HSCs, quantitative PCR (qPCR) was performed for each candidate gene using LSCs derived from BM of 5 AML patients and HSCs derived from BM of 4 (Table 3). Out of the 217 genes identified, 121 genes that encode molecules in the following categories were selected as candidates best suiting for the development of pharmaceuticals, and subjected to subsequent analysis. The three categories are as follows:

1) those located on the cell membrane or in extracellular spaces,
2) cytokines, growth factors, transmembranous receptors, protein kinases, phosphatases, transcriptional regulation molecules, and/or signaling molecules, and
3) those involved in immune regulation, cell cycle, apoptosis, and/or cell adhesion.

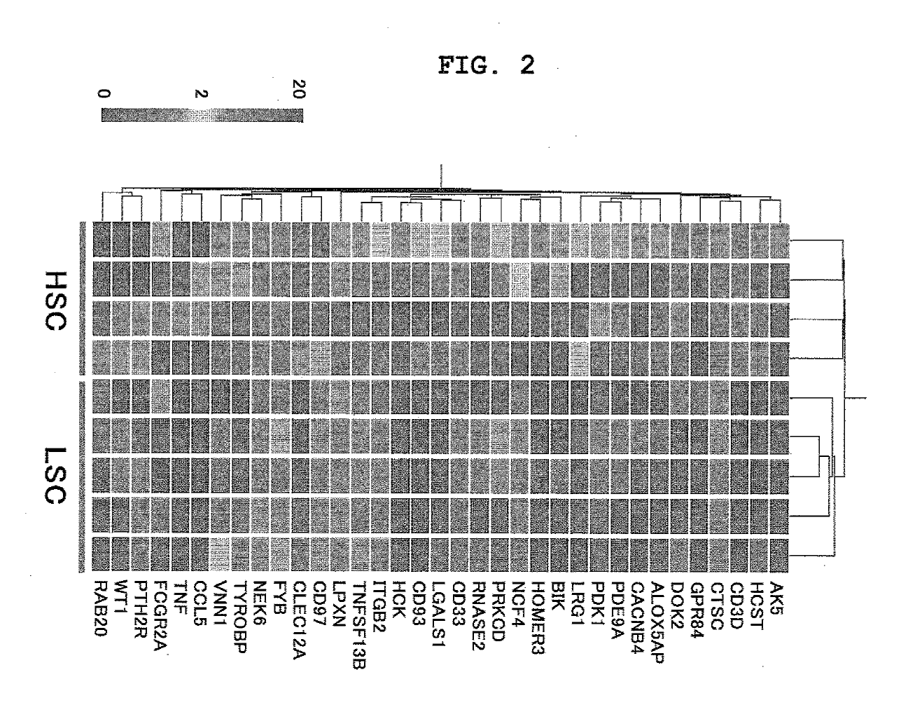

As shown in Table 2, the mRNA contents concerning 57 genes out of the 121 genes were statistically higher in LSCs than in HSCs. Of the 57 genes, 35 genes were identified as excellent LSC markers. The reason was that 1) the median expression levels of these genes were 5 times or higher in LSCs, and that 2) their mRNA contents were higher in all LSC samples tested than in each HSC population tested (FIG. 2).

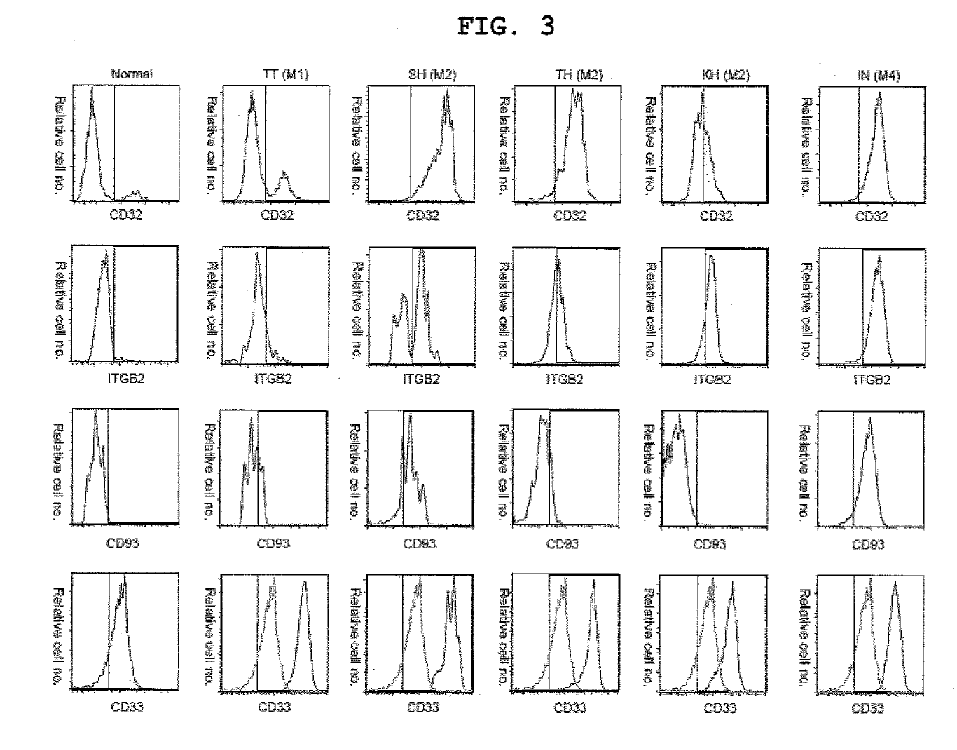

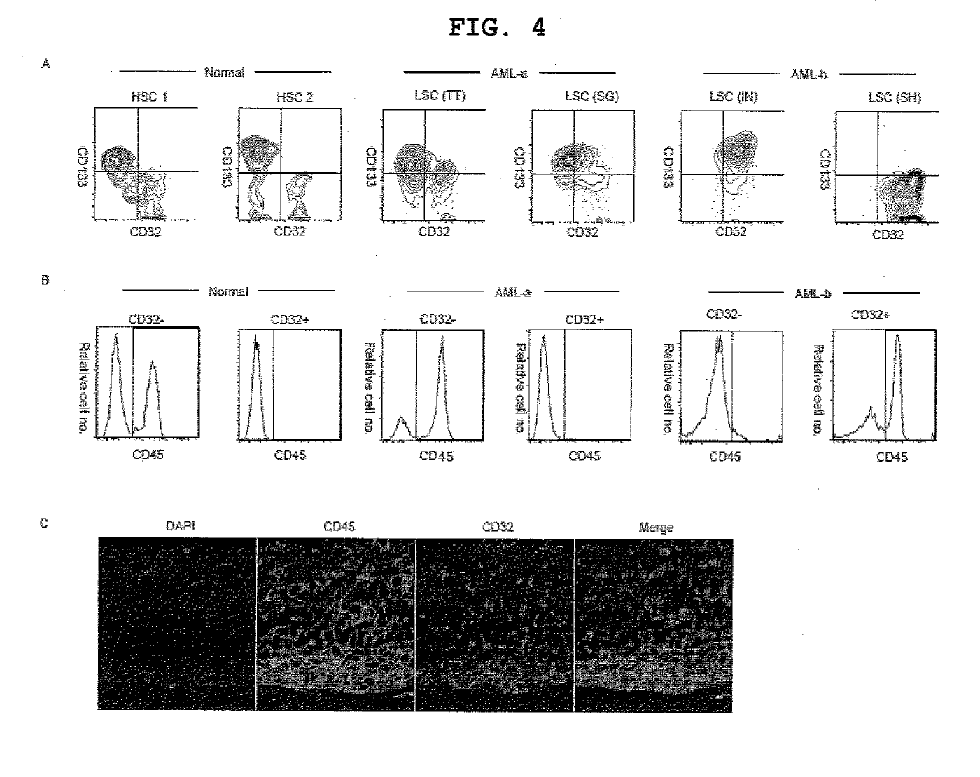

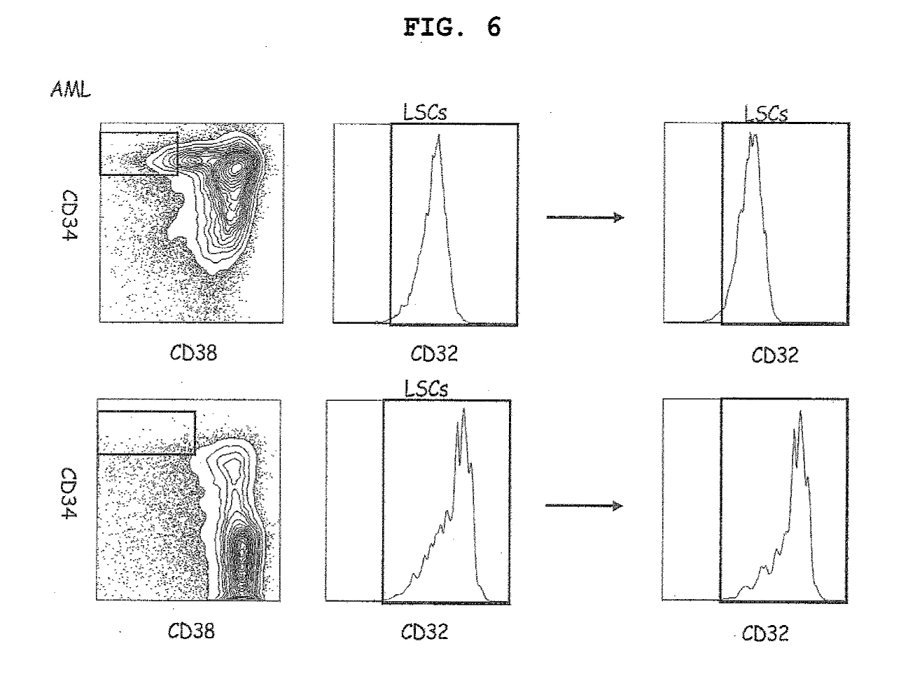

To confirm the expression of these LSC-specific candidate molecules at the protein level, the quality of monoclonal antibodies and polyclonal antibodies that can be utilized for the 35 candidate molecules, respectively, was verified, and flow cytometric analysis was performed using antibodies proven to be effective and 32 AML patient samples. Through the flow cytometric analysis, the following aspects were examined in each candidate molecule: 1) localization (on cell surfaces or in cells), 2) frequency of positive cells, and 3) expression intensity. Out of the 57 candidate molecules thus assessed, FCGR2A(CD32), ITGB2(CD18), CD93, CD33, CD3D and TNF(TNFa) were found to have the most promising expression level/pattern for LSC-specific markers/targets. In particular, the expression of FCGR2A(CD32) exhibited a strong correlation with LSCs in a significant ratio of the AML patients tested, and this was selected for further functional analysis. In 9 of the 32 AML patients tested, the great majority (>80%) of AML stem cells expressed this antigen (FIG. 3). To confirm that the expression of CD32 correlates exclusively with the function, in vivo NOD/SCID/IL2rg KO transplantation assay was performed using purified LSCs derived from three patients with AML. When purified CD34+CD38−CD32+ and CD34+CD38−CD32− cells were transplanted to sub-lethally irradiated recipients, AML developed exclusively from the CD32+ fraction (FIG. 4). Because any LSC-targeting treatment is thought to be best used along with a commonly used chemotherapeutic agent that is effective in removing non-LSC AML cells, it is important to confirm that the target molecule is continuously expressed even after chemotherapy. Accordingly, the present inventors examined whether the expression of CD32 was maintained after chemotherapy, and confirmed the expression of CD32 in BM, spleens and peripheral blood (PB) of AML transplantation recipient mice after AraC treatment (FIG. 6). Also, CD32-expressing cells were found by immunofluorescent labeling in both the membrane region and central region of bone marrow (FIG. 4). This finding, in view of the previous report by the present inventors that chemotherapy-resistant LSCs are present in BM osteoblast niches, further supports CD32 as a candidate for LSC target therapy (Ishikawa F. et al. Nature Biotechnol 25:1315-1321, 2007 and PCT/JP2008/068892).

Next, the expression of CD32 in normal human HSCs was assessed. In the primary human CB CD34+CD38− population, the frequency of CD32+ cells was 9.8%+/−SD (FIG. 4A). When the expression of CD133 in this fraction was analyzed, CD32+ cells were detected exclusively in the CD34+CD38−CD133− fraction (FIG. 4a). It was found by heterologous transplantation assay that not the CD34+CD38−CD32+ fraction but the CD34+CD38−CD133+CD32− fraction contains HSCs (FIG. 4B). Furthermore, it was suggested by in vitro colony-forming cell (CFC) assay that not CD34+CD38−CD32+ cells but CD34+CD38−CD32− cells have the capability of producing bone marrow-series and erythrocyte-series hematopoietic colonies. The lack of the capability of in vivo long-term hematopoiesis reconstitution in CD32+ normal HSCs suggests the possibility that therapeutic agents targeting CD32 expression cells may help avoid important adverse reactions related to the normal hematopoietic and immune systems without affecting HSCs.

The present inventors first confirmed by neonatal NOD/SCID/IL2rg KO mouse transplantation assay that in AML patient samples lacking the expression of CD32 by LSCs, CD34+CD38− cells possess the LSC function (FIG. 1), and then examined the expression of ITGB2(CD18), CD93, (as well as CD25, CD132, OX41, and CD97), CD33, CD3D and TNFα by flow cytometry. Combination of the antigens CD32, ITGB2, CD93, 97 and 33 enabled good separation of LSCs from normal HSCs in 31 patients out of 47 patients.

The list of LSC-specific genes identified using the two sets of microarrays and quantitative PCR (Table 2) includes genes that are expressed preferentially in bone marrow progeny, but their expression is limited in HSCs. For example, FCGR2A, HCK and NCF4 are highly expressed in mature bone marrow cells and mediate the phagocytosis by immunoconjugates and subsequent superoxide production (Prot Natl Acad Sci USA, 97, 1725; 2000; J Exp Med 191, 669, 2000; Nat Cell Biol, 3, 679, 2001; J Biol Chem 279, 1415, 2004). Meanwhile, CD3D, which is a constituent of the CD3 conjugate, transmits in mature T lymphocytes a T cell receptor signal via the ITAM motif thereof. Therefore, at least a particular ratio of AMLs can develop via abnormal regulation of differentiation in the stem cell stage.

Another feature of the list is the involvement of genes expressed remarkably in cancer cells and leukemia cells. For example, CD33 is a well recognized immunological marker of AML cells, and serves as a target for antibody pharmaceuticals such as gemtuzumab ozogamicin (Leukemia 19: 176, 2005). Furthermore, CD97 has been reported to be accumulated in colorectal cancers that infiltrate lymphatic vessels (Am Pathol 161, 1657, 2002). Overexpression of these molecules in LSCs suggests that a therapeutic method that targets these molecules may be effective not only on LSCs, but also on mature AML cells. It should be noted that gene products of BIK, HOMER3, WT1 (Genes Chromosomes Cancer 47, 8-20, 2008) and CLEC12A (encoding CLL-1) (Blood 110, 2659-2666, 2007) have been proposed as marker molecules for LSC/AML blasts, and this demonstrates that the findings of the present invention agree with available reports.

By analyzing the expression levels/patterns, the candidate genes were classified into the following sets:

1) a set of genes that encode molecules expressed in a significant ratio of LSCs at the RNA and protein levels, but expressed in only a small ratio of HSCs (or not expressed), and
2) a set of genes expressed at the protein level in LSCs and HSCs, but whose expression intensity as determined by flow cytometry allows separation of LSCs from HSCs.

The gene set 1 includes candidates that specifically target LSCs and do not affect HSCs, for example, promising candidates for the development of therapeutic agents such as antibody pharmaceuticals based on the lack of the aforementioned candidates in normal HSCs. The genes included in the gene set 2 (the most promising candidate is CD33) encode biomarkers having high applicability to ex vivo purging of LSCs for separating LSCs from HSCs and the like against the background of autologous transplantation of hematopoietic stem cells.

As shown in FIG. 7, it was found by identifying the location and the phase in cell cycle by imaging using an antibody against each marker (FCGR2A, AK5, DOK2, LRG1, BIK, IL2RA, WT1 and SUCNR1) and a stationary cell-specific marker, that these molecules are abundantly present in the endosteum (niches), where stem cells exhibiting anticancer agent resistance are present, and are expressed in leukemic stem cells while in the stationary phase of cell cycle. Therefore, targeting these individual marker molecules is thought to be largely contributory to overcoming recurrences of leukemia.

WT1 has been shown to be expressed in a wide variety of tumors, including leukemia. However, whether this molecule is expressed at the level of stem cells, which exhibit recurrences and anticancer agent resistance, has been unknown. The present inventors found that this molecule is expressed in leukemic stem cells that are present in niches and are in the stationary phase of cell cycle, and have shown that the molecule is of significance as a target molecule for killing leukemic stem cells, which have been unable to be killed by conventional chemotherapy and radiotherapy.

Also, peripheral blood was collected from 47 patients with AML in various stages, samples containing hematopoietic cells were prepared, and FCGR2A(CD32a), FCGR2B(CD32b), IL2RA(CD25), ITGB2(CD18) and CD93 positively rates in leukemic stem cells contained in the samples were examined. The results are shown in Table 4.

TABLE 4

| | | Any | | | | | |
|---|---|---|---|---|---|---|---|
| | n | marker | CD32-a | CD32-b | CD25 | CD18 | CD93 |
| AML M0 | 2 | 2 | 2 | 0 | 0 | 0 | 0 |
| % marker positive | | 100.0 | 100.0 | 0.0 | 0.0 | 0.0 | 0.0 |
| AML M1 | 7 | 4 | 0 | 2 | 2 | 3 | 0 |
| % marker positive | | 57.1 | 0.0 | 28.6 | 28.6 | 42.9 | 0.0 |
| AML M2 | 14 | 9 | 5 | 4 | 4 | 5 | 1 |
| % marker positive | | 64.3 | 35.7 | 28.6 | 28.6 | 35.7 | 7.1 |
| AML M4 | 4 | 4 | 3 | 1 | 1 | 2 | 1 |
| % marker positive | | 100.0 | 75.0 | 25.0 | 25.0 | 50.0 | 25.0 |
| Other AML | 3 | 1 | 1 | 0 | 0 | 0 | 0 |
| % marker positive | | 33.3 | 33.3 | 0.0 | 0.0 | 0.0 | 0.0 |
| MDS/AML | 17 | 11 | 3 | 6 | 9 | 0 | 1 |
| % marker positive | | 64.7 | 17.6 | 35.3 | 52.9 | 0.0 | 5.9 |
| All cases | 47 | 31 | 14 | 13 | 16 | 10 | 3 |
| % marker positive | | 66.0 | 29.8 | 27.7 | 34.0 | 21.3 | 6.4 |

From Table 4, it is seen that by combining 4 kinds of markers FCGR2A(CD32a), IL2RA(CD25), ITGB2(CD18) and CD93, leukemic stem cells can be distinguished at a high rate, and by combining pharmaceuticals that target these 4 kinds of genes, over 60% of leukemia cells can be exterminated.

### INDUSTRIAL APPLICABILITY

By using a leukemic stem cell marker found in the present invention as a molecular target, a therapeutic agent that acts specifically on. LSCs that are the source of onset or recurrence of AML can be provided. It is possible to specifically remove LSCs from bone marrow cells of a patient or a donor using a cell sorter such as FACS, with a leukemic stem cell marker found in the present invention as an index. This will increase effectiveness of purging for autologous or allogeneic bone marrow transplantation, and enable to significantly prevent recurrences or the initial onset of acute myeloid leukemia. Furthermore, the presence or absence of LSCs in a collected biological sample or in a living organism can be determined with a leukemic stem cell marker found in the present invention as an index, whereby recurrences or the initial onset of acute myeloid leukemia can also be predicted.

This application is based on a patent application No. 2009-072400 filed in Japan (filing date: Mar. 24, 2009), the contents of which are incorporated in full herein.

1. A test method for predicting the initial onset or a recurrence of acute myeloid leukemia, comprising
(1) a step of measuring the expression level of leukemic stem cell marker genes in a biological sample collected from a subject for a transcription product or translation product of the genes as an analyte, and
(2) a step of comparing the expression levels obtained in the measuring step with a reference value;
wherein the leukemic stem cell marker genes are 2-218 genes selected from the group consisting of:
cell membrane- or extracellularly-localized genes consisting of ADFP, ALOX5AP, AZU1, C3AR1, CACNB4, CALCRL, CCL4, CCL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, IL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT;
apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C17orf60, C18 orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, S100P, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT1, WSB2 and ZNF253;
and wherein when the expression of two or more leukemic stem cell marker genes in the subject is significantly higher than the reference value, a possible presence of a leukemic stem cell in the collected biological sample or the subject's body is suggested.

2. The test method according to claim 1, wherein the leukemic stem cell marker genes are 2-58 genes selected from the group consisting of:
cell membrane- or extracellularly-localized genes consisting of ADFP, ALOX5AP, CACNB4, CCL5, CD33, CD3D, CD93, CD97, CLEC12A, DOK2, FCER1G, FCGR2A, FUCA2, GPR34, GPR84, HCST, HGF, HOMER3, IL2RA, IL2RG, IL3RA, ITGB2, LGALS1, LRG1, LY86, MGAT4A, P2RY5, PRSS21, PTH2R, RNASE2, SLC43A3, SUCNR1, TIMP1, TNF, TNFRSF4, TNFSF13B, TYROBP and VNN1; cell cycle-related genes consisting of ZWINT, NEK6 and TXNL4B; an apoptosis-related gene consisting of BIK; signaling-related genes consisting of AK5, ARHGAP18, FYB, HCK, LPXN, PDE9A, PDK1, PRKCD, RAB20, RAB8A and RABIF; transcription factor genes consisting of WT1 and HLX; and other genes consisting of CYBB, CTSC and NCF4.

3. A therapeutic agent for acute myeloid leukemia that targets leukemic stem cells, comprising as an active ingredient a substance capable of suppressing the expression of a gene selected from among leukemic stem cell marker genes consisting of the following set of genes:
cell membrane- or extracellularly-localized genes consisting of ADFP, ALOX5AP, AZU1, C3AR1, CACNB4, CALCRL, CCL4, CCL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, IL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT;
apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C17orf60, C18orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, S100P, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT1, WSB2 and ZNF253;
or a substance capable of suppressing the activity of a translation product of the gene.

4. The therapeutic agent according to claim 3, wherein the leukemic stem cell marker gene is selected from the group consisting of:
cell membrane- or extracellularly-localized genes consisting of ADFP, ALOX5AP, CACNB4, CCL5, CD33, CD3D, CD93, CD97, CLEC12A, DOK2, FCER1G, FCGR2A, FUCA2, GPR34, GPR84, HCST, HGF, HOMER3, IL2RA, IL2RG, IL3RA, ITGB2, LGALS1, LRG1, LY86, MGAT4A, P2RY5, PRSS21, PTH2R, RNASE2, SLC43A3, SUCNR1, TIMP1, TNF, TNFRSF4, TNFSF13B, TYROBP and VNN1; cell cycle-related genes consisting of ZWINT, NEK6 and TXNL4B; an apoptosis-related gene consisting of BIK; signaling-related genes consisting of AK5, ARHGAP18, FYB, HCK, LPXN, PDE9A, PDK1, PRKCD, RAB20, RAB8A and RABIF; transcription factor genes consisting of WT1 and HLX; and other genes consisting of CYBB, CTSC and NCF4.

5. The therapeutic agent according to claim 3, wherein the leukemic stem cell marker gene is selected from the group consisting of:
cell membrane- or extracellularly-localized genes consisting of ALOX5AP, CACNB4, CCL5, CD33, CD3D, CD93, CD97, CLEC12A, DOK2, FCGR2A, GPR84, HCST, HOMER3, ITGB2, LGALS1, LRG1, PTH2R, RNASE2, TNF, TNFSF13B, TYROBP and VNN1; a cell cycle-related gene consisting of NEK6; an apoptosis-related gene consisting of BIK; signaling-related genes consisting of AK5, FYB, HCK, LPXN, PDE9A, PDK1, PRKCD and RAB20; a transcription factor gene consisting of WT1; and other genes consisting of CTSC and NCF4.

6. The therapeutic agent according to claim 3, wherein the leukemic stem cell marker gene is a marker expressed in stem cells that are present in bone marrow niches, are in the stationary phase of cell cycle, and are resistant to anticancer agents, selected from the group consisting of AK5, BIK, DOK2, FCGR2A, IL2RA, LRG1, SUCNR1 and WT1.

7. The therapeutic agent according to claim 3, wherein the substance capable of suppressing the expression of the gene is an antisense nucleic acid or an RNAi-inducible nucleic acid.

8. The therapeutic agent according to claim 3, wherein the substance capable of suppressing the activity of the translation product is an aptamer or an antibody.

9. The therapeutic agent according to claim 8, wherein the antibody is an immunoconjugate of an antibody and an anticancer substance.

10. A production method of a sample containing hematopoietic cells for autologous transplantation or allogeneic transplantation for a patient with acute myeloid leukemia, comprising:
a) a step of collecting a sample containing hematopoietic cells from the patient or a donor;
b) a step of bringing the collected sample into contact with a substance that recognizes a translation product of at least one kind of leukemic stem cell marker gene selected from among the following set of genes:
cell membrane- or extracellularly-localized genes consisting of ADFP, ALOX5AP, AZU1, C3AR1, CACNB4, CALCRL, CCL4, CCL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, IL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT;
apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C17orf60, C18 orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, SLOOP, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT1, WSB2 and ZNF253; and
c) a step of sorting cells to which the substance has bound, and obtaining the sample from which leukemic stem cells have been purged.

11. The production method according to claim 10, wherein the leukemic stem cell marker is at least one kind of cell surface marker gene selected from among ADFP, ALOX5AP, CACNB4, CD33, CD3D, CD93, CD97, CLEC12A, DOK2, FCER1G, FCGR2A, GPR34, GPR84, HCST, HOMER3, IL2RA, IL2RG, IL3RA, ITGB2, LY86, P2RY5, PTH2R, SUCNR1, TNFRSF4, TYROBP and VNN1.

12. A method for preventing or treating acute myeloid leukemia that targets leukemic stern cells, comprising administering, to a subject, an effective amount of a substance capable of suppressing the expression of a gene selected from among leukemic stern cell marker genes consisting of the following set of genes:
cell membrane- or extracellularly-localized genes consisting of ADFP, ALOX5AP, AZU1, C3AR1, CACNB4, CALCRL, CCL4, CCL5, CD33, CD36, CD3D, CD86, CD9, CD93, CD96, CD97, CFD, CHI3L1, CLEC12A, CLECL1, COCH, CST7, CXCL1, DOK2, EMR2, FCER1G, FCGR2A, FUCA2, GPR109B, GPR160, GPR34, GPR84, HAVCR2, HBEGF, HCST, HGF, HLA-DOB, HOMER3, IFI30, IL13RA1, IL2RA, IL2RG, IL3RA, INHBA, ITGB2, LGALS1, LRG1, LY86, MAMDC2, MGAT4A, P2RY14, P2RY5, PLAUR, PPBP, PRG2, PRSS21, PTH2R, PTX3, REEP5, RNASE2, RXFP1, SLC31A2, SLC43A3, SLC6A6, SLC7A6, STX7, SUCNR1, TACSTD2, TIMP1, TM4SF1, TM9SF1, TNF, TNFRSF4, TNFSF13B, TYROBP, UTS2 and VNN1;
cell cycle-related genes consisting of AURKA, C13orf34, CCNA1, DSCC1, FAM33A, HPGD, NEK6, PYHIN1, RASSF4, TXNL4B and ZWINT;
apoptosis-related genes consisting of MPO, IER3, BIK, TXNDC1, GADD45B and NAIP;
signaling-related genes consisting of AK5, ARHGAP18, ARRB1, DUSP6, FYB, HCK, LPXN, MS4A3, PAK1IP1, PDE9A, PDK1, PRKAR1A, PRKCD, PXK, RAB20, RAB8A, RABIF, RASGRP3, RGS18 and S100A11;
transcription factor genes consisting of WT1, MYC and HLX; and
other genes consisting of ACTR2, ALOX5, ANXA2P2, ATL3, ATP6V1B2, ATP6V1C1, ATP6V1D, C12orf5, C7orf60, C18orf19, C1GALT1C1, C1orf135, C1orf163, C1orf186, C6orf150, CALML4, CCT5, CLC, COMMD8, COTL1, COX17, CRIP1, CSTA, CTSA, CTSC, CTSG, CYBB, CYP2E1, DENND3, DHRS3, DLAT, DLEU2, DPH3, EFHD2, ENC1, EXOSC3, FAM107B, FAM129A, FAM38B, FBXO22, FLJ14213, FNDC3B, GNPDA1, GRPEL1, GTSF1, HIG2, HN1, HVCN1, IDH1, IDH3A, IKIP, KIF2C, KYNU, LCMT2, ME1, MIRN21, MKKS, MNDA, MTHFD2, MYO1B, MYO1F, NAGA, NCF2, NCF4, NDUFAF1, NP, NRIP3, OBFC2A, PARP8, PDLIM1, PDSS1, PGM2, PIGK, PIWIL4, PPCDC, PPIF, PRAME, PUS7, RPP40, RRM2, S100A16, S100A8, S100P, S100Z, SAMHD1, SH2D1A, SPCS2, SPPL2A, TESC, THEX1, TMEM30A, TMEM33, TRIP13, TUBB6, UBASH3B, UGCG, VSTM1, WDR4, WIT1, WSB2 and ZNF253;
or a substance capable of suppressing the activity of a translation product of the gene.

13. The therapeutic agent according to claim 4, wherein the substance capable of suppressing the expression of the gene is an antisense nucleic acid or an RNAi-inducible nucleic acid.

14. The therapeutic agent according to claim 4, wherein the substance capable of suppressing the activity of the translation product is an aptamer or an antibody.

15. The therapeutic agent according to claim 14, wherein the antibody is an immunoconjugate of an antibody and an anticancer substance.

16. The therapeutic agent according to claim 5, wherein the substance capable of suppressing the expression of the gene is an antisense nucleic acid or an RNAi-inducible nucleic acid.

17. The therapeutic agent according to claim 5, wherein the substance capable of suppressing the activity of the translation product is an aptamer or an antibody.

18. The therapeutic agent according to claim 17, wherein the antibody is an immunoconjugate of an antibody and an anticancer substance.

19. The therapeutic agent according to claim 6, wherein the substance capable of suppressing the expression of the gene is an antisense nucleic acid or an RNAi-inducible nucleic acid.

20. The therapeutic agent according to claim 6, wherein the substance capable of suppressing the activity of the translation product is an aptamer or an antibody.

21. The therapeutic agent according to claim 20, wherein the antibody is an immunoconjugate of an antibody and an anticancer substance.
